(19) United States
(12) Patent Application Publication
(10) Pub. No.: US 2025/0177965 A1
(43) Pub. Date: Jun. 5, 2025

(54) HETEROGENEOUS CATALYSTS AND METHODS MAKING AND USING THEREOF

(71) Applicant: The University of Hong Kong, Hong Kong (CN)

(72) Inventors: Jian He, Hong Kong (CN); Jun Guo, Hong Kong (CN); Qi Xia, Hong Kong (CN)

(21) Appl. No.: 18/957,062

(22) Filed: Nov. 22, 2024

Related U.S. Application Data
(60) Provisional application No. 63/604,642, filed on Nov. 30, 2023.

(51) Int. Cl. B01J 31/16; B01J 35/39; B01J 37/00

(52) U.S. Cl. CPC B01J 31/1691; B01J 35/39; B01J 37/00; B01J 2231/324

(57) ABSTRACT

Described herein are heterogeneous catalysts and methods of making and using thereof. For instance, heterogeneous catalysts formed of bisphosphine-ligated copper(I) complexes on a metal-organic framework support can be used, for example, in cycloaddition reactions.

### CROSS-REFERENCE TO RELATED APPLICATIONS

This application claims the benefit of and priority to U.S. Provisional Application No. 63/604,642 filed on Nov. 30, 2023, the contents of which is incorporated herein in its entirety.

### FIELD OF THE INVENTION

The disclosed invention is generally in the field of heterogeneous catalysts which can be used, for example, in cycloaddition reactions.

### BACKGROUND OF THE INVENTION

Construction of aryl-substituted cyclobutane rings, the prominent structural features in a vast array of bioactive molecules (Goetzke, F. W., Hell, A. M. L., van Dijk, L. & Fletcher, S. P. A catalytic asymmetric cross-coupling approach to the synthesis of cyclobutanes. Nat. Chem. 13, 880-886 (2021); Dembitsky, V. M. Naturally occurring bioactive cyclobutane-containing (CBC) alkaloids in fungi, fungal endophytes, and plants. Phytomedicine 21, 1559-1581 (2014)), is essential for natural product synthesis and drug discovery (Namyslo, J. C. & Kaufmann, D. E. The application of cyclobutane derivatives in organic synthesis. Chem. Rev. 103, 1485-1538 (2003); Wang, M. & Lu, P. Catalytic approaches to assemble cyclobutane motifs in natural product synthesis. Org. Chem. Front. 5, 254-259 (2018)).

Photochemical [2+2]cycloadditions are arguably the most straightforward way to prepare such cyclobutane-containing compounds, and rely primarily on the activation of styrenes via electron or energy transfer with transition-metal or organic photosensitizers (Poplata, S., Tröster, A., Zou, Y.-Q. & Bach, T. Recent advances in the synthesis of cyclobutanes by olefin [2+2] photocycloaddition reactions. Chem. Rev. 116, 9748-9815 (2016); Zhou, Q.-Q., Zou, Y.-Q., Lu, L.-Q. & Xiao, W.-J. Visible-light-induced organic photochemical reactions through energy-transfer pathways. Angew. Chem. Int. Ed. 58, 1586-1604 (2019); Sicignano, M., Rodríguez, R. I. & Alemán, J. Recent visible light and metal free strategies in [2+2] and [4+2]photocycloadditions. Eur. J. Org. Chem. 2021, 3303-3321 (2021); Zhu, M., Zhang, X., Zheng, C. & You, S.-L. Energy-transfer-enabled dearomative cycloaddition reactions of indoles/pyrroles via excited-state aromatics. Acc. Chem. Res. 55, 2510-2525 (2022)). The same type of oxidative intermolecular crossed [2+2]cycloadditions have also been achieved using various polymer-based heterogeneous photocatalysts (Li, R. et al. Photocatalytic regioselective and stereoselective [2+2]cycloaddition of styrene derivatives using a heterogeneous organic photocatalyst. ACS Catal. 7, 3097-3101 (2017); Piane, J. J. et al. Organic photoredox-catalyzed cycloadditions under single-chain polymer confinement. ACS Catal. 10, 13251-13256 (2020)). While the Yoon group reported the first intramolecular crossed [2+2]cycloadditions of 1,2-disubstituted styrenes through triplet-energy-transfer catalysis in 2012(Lu, Z. & Yoon, T. P. Visible light photocatalysis of [2+2] styrene cycloadditions by energy transfer. Angew. Chem. Int. Ed. 51, 10329-10332 (2012)), intermolecular [2+2]cycloadditions between different styrenes involving triplet styrene formation were not realized until very recently(Liu, Z. et al. Aggregation-enabled intermolecular photo[2+2]cycloaddition of aryl terminal olefins by visible-light catalysis. CCS Chem. 2, 582-588 (2020)). In terms of heterogeneous photocatalytic energy transfer, only quantum dots are reported to be capable of promoting styrene [2+2]cycloadditions (Jiang, Y., Wang, C., Rogers, C. R., Kodaimati, M. S. & Weiss, E. A. Regio- and diastereoselective intermolecular [2+2]cycloadditions photocatalysed by quantum dots. Nat. Chem. 11, 1034-1040 (2019))..

Despite substantial advances in triplet photosensitization over the past decades, developing intermolecular crossed [2+2]cycloadditions of simple styrenes with other alkenes still remains a significant challenge using visible light photocatalysis.

Accordingly, there remains a need for improved catalysts which can address the aforementioned issues for use in cycloaddition reactions.

Therefore, it is an object of the present invention to provide heterogeneous catalysts with improved properties.

It is a further object of the present invention to provide methods of making such heterogeneous catalysts.

It is still a further object of the present invention to provide methods of using such heterogeneous catalysts to efficiently drive cycloaddition reactions.

### SUMMARY OF THE INVENTION

Heterogeneous catalysts which can be used in chemical reactions, such as [2+2]cycloadditions are described herein. In one instance, a non-limiting example a heterogenous catalyst includes:
a metal-organic framework formed of a plurality of inorganic nodes and a plurality of organic linkers wherein at least one organic linker of the plurality comprises a phenanthroline moiety;
wherein at least one copper (I) ion is ligated to the phenanthroline moiety and ligated to a phosphine ligand forming a catalyst complex within the metal-organic framework.

In certain instances, the catalyst complex within the metal-organic framework includes a chemical structure as follows:

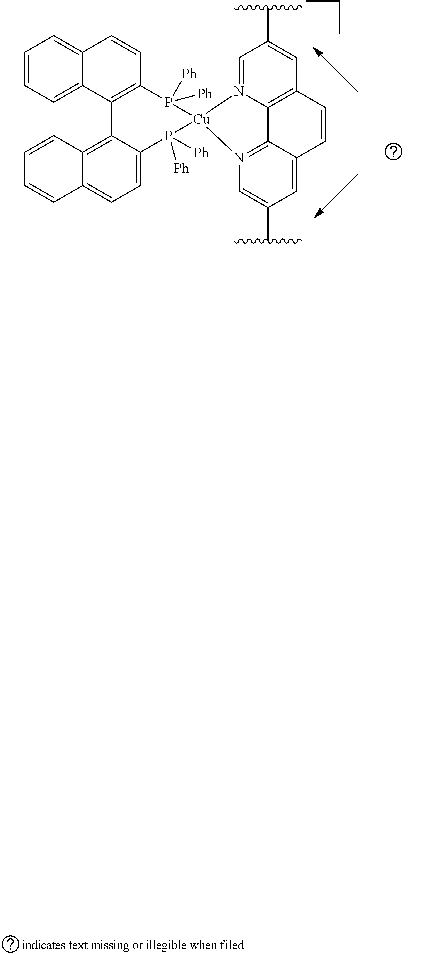

where the wavy bonds extending from the phenanthroline moiety are understood by the skilled artisan to be part of an organic linker containing the moiety within the metal-organic framework.

The heterogeneous catalysts (i.e., heteroleptic copper(I) complexes with bidentate nitrogen and phosphorus donor ligands) function as photoredox catalysts and (high) triplet photosensitizers, which can be used for a wide range of light-mediated organic transformations. The heterogeneous catalysts can provide high turnover numbers (TON) in a range of about 50 to 5000 turnovers, as well as sub-ranges or individual values contained within the aforementioned range.

The heterogeneous catalysts typically have relatively long photoexcited-state lifetimes. For instance, the heterogeneous catalysts can have an excited-state lifetime at room temperature of at least about 3, 3.5, 4, 4.5, or 5 μs; or an excited-state lifetime at room temperature in a range of between about 3 to 5 μs, as well as sub-ranges or individual values contained within the aforementioned range.

The heterogeneous catalysts described can be synthesized by various methods. In one instance, a non-limiting method of synthesizing a heterogenous catalyst includes the steps of:
(i) reacting a plurality of organic linkers, wherein at least one organic linker of the plurality includes a phenanthroline moiety, with an inorganic salt to form a metal-organic framework comprising a plurality of inorganic nodes;
(ii) metalating the metal-organic framework by mixing a metal complex comprising at least one copper (I) ion ligated to a phosphine ligand with the metal-organic framework;
where the metalating step includes the least one copper (I) ion becoming ligated to the phenanthroline moiety to form a catalyst complex within the metal-organic framework.

In another non-limiting instance, a method of synthesizing a heterogenous catalyst includes the steps of:
(i′) metalating a metal-organic framework by mixing a metal complex comprising at least one copper (I) ion ligated to a phosphine ligand with the metal-organic framework;
where the metal-organic framework is formed of a plurality of inorganic nodes and a plurality of organic linkers wherein at least one organic linker of the plurality includes a phenanthroline moiety;
where the metalating step comprises the least one copper (I) ion becoming ligated to the phenanthroline moiety to form a catalyst complex within the metal-organic framework.

The heterogeneous catalysts described below can be used in large-scale synthesis and demonstrate catalyst recyclability, as well as facilitate the separation of catalyst and product. Moreover, the heterogeneous catalysts can be employed to achieve organic transformations that are not known to be accomplished in previously established photocatalytic systems, such as intermolecular [2+2]cycloadditions of styrenes with electron-deficient olefins.

### BRIEF DESCRIPTION OF THE DRAWINGS

FIG. 1A shows two homogeneous copper catalysts (left and center) and a non-limiting example of heterogeneous copper catalyst supported by a metal-organic framework (MOF).

FIG. 1B shows two non-limiting reactions which are possible using heterogeneous catalysts and which demonstrate high catalytic efficiency and broad substrate scope.

FIG. 2A shows a non-limiting synthetic scheme of the synthesis of a UiO-69-type copper-based heterogenous catalyst.

FIG. 2B shows a comparison of PXRD patterns of UiO-69-phen(binap)Cu, UiO-69-phen(xantphos)Cu, UiO-69-phenCu, and UiO-69-phen with simulations of UiO-69 MOF.

FIG. 2C shows Cu2p XPS spectra showing the +1 oxidation state of the Cu centers in UiO-69-phen(binap)Cu, UiO-69-phen(xantphos)Cu, and UiO-69-phenCu.

FIG. 2D shows an EXAFS spectrum and fitting data in R-space at the Cu K-edge absorption of UiO-69-phen(binap)Cu. χ is the fine structure function. R is the interatomic distance from Cu.

FIG. 3 shows the chemical structures of compounds Cu-1, Cu-2, Cu-3, and Cu-4.

FIG. 4 shows a substrate scope study of crossed [2+2]cycloaddition of styrenes with electron-deficient alkenes. Reaction conditions: styrene (0.2 mmol, 1.0 equiv.), electron-deficient alkene (3.0 or 5.0 equiv.), UiO-69-phen(binap)Cu (0.2 mol %), and DMAP (1.2 equiv.) in anhydrous DCE (1 ml) under nitrogen atmosphere at room temperature with blue-LED light irradiation (440 nm) for 48 h. For each entry number (in bold), data are reported as isolated yields. Diastereomeric ratios were determined by ¹H NMR analysis of the crude reaction mixture. ^{a}With 0.75 mol % catalyst. ^{b}Dimethyl fumarate (1.2 equiv.) was used. ^{c}With 1.5 mol % catalyst. For experimental details, see Example 1. ^{d}With 0.5 mol % catalyst. DMAP, 4-dimethylaminopyridine; DCE, 1,2-dichloroethane; tBu, tert-butyl; Bn, benzyl; Bpin, (pinacolato)boryl.

FIG. 5 shows a substrate scope study of a [2+2]cycloaddition between different styrenes. Reaction conditions: styrene (2.0 mmol, 1.0 equiv.), the other styrene (5.0 equiv.), UiO-69-phen(binap)Cu (0.02 mol %), and DMAP (1.2 equiv.) in anhydrous DCE (1 ml) under nitrogen atmosphere at room temperature with blue-LED light irradiation (440 nm) for 48 h. For each entry number (in bold), data are reported as isolated yields. Diastereomeric ratios were determined by ¹H NMR analysis of the crude reaction mixture. DMAP, 4-dimethylaminopyridine; DCE, 1,2-dichloroethane; Ac, acetyl; ⁱPr, isopropyl.

FIG. 6 shows a substrate scope study of crossed [2+2]cycloaddition of exocyclic arylidene azetidines, thietanes, and oxetanes with electron-deficient alkenes and styrenes. Reaction conditions: exocyclic arylidene azetidine, thietane, or oxetane (0.2 mmol, 1.0 equiv.), electron-deficient alkene or styrene (3.0 equiv.), UiO-69-phen(binap)Cu (0.2 mol %), and DMAP (1.2 equiv.) in anhydrous DCE (1 ml) under nitrogen atmosphere at room temperature with blue-LED light irradiation (440 nm) for 48 h. For each entry number (in bold), data are reported as isolated yields. Diastereomeric ratios were determined by ¹H NMR analysis of the crude reaction mixture. ^{a}With 0.75 mol % catalyst. ^{b}With 1 mol % catalyst. ^{c}With 0.5 mol % catalyst. ^{d}The diastereomeric ratio was determined after purification by silica gel column chromatography. DMAP, 4-dimethylaminopyridine; DCE, 1,2-dichloroethane; Boc, tert-butoxycarbonyl; Bn, benzyl; Ac, acetyl; Bpin, (pinacolato)boryl.

FIG. 7A shows photostability testing parameters and results of binap-ligated copper species in compound Cu-2.

FIG. 7B shows photostability testing parameters and results of binap-ligated copper species in UiO-69-phen(binap)Cu.

FIG. 7C shows a graph of recycling experiments for crossed [2+2]cycloaddition of 1-vinylnaphthalene with acrylonitrile showing yields over six catalytic runs.

FIG. 7D shows a comparison of PXRD patterns of the original and recycled UiO-69-phen(binap)Cu.

FIG. 8A shows solid-state emission spectra (excitation wavelength: 420 nm) of UiO-69-phen(binap)Cu and Cu-1 recorded under argon at room temperature.

FIG. 8B shows a proposed ruling out of electron-transfer pathways.

FIG. 8C shows two triplet excited-state quenching experiments.

FIG. 8D shows a proposed mechanism for a heterogeneous photocatalytic [2+2]cycloaddition.

FIG. 9 shows thermogravimetric (TGA) plots of various UiO-69-type materials under N₂ atmosphere. The percentages denote the amount of weight loss at 200° C.

### DETAILED DESCRIPTION OF THE INVENTION

Heterogeneous catalysts and methods of making and using thereof are described herein.

### I. Definitions

An “aryl radical” or “aryl group” is understood to mean a radical containing a structure made up of 6 to 30 carbon atoms, 6 to 18 carbon atoms, which is formed from one aromatic ring or a plurality of fused aromatic rings. Exemplary aryl radicals are, without limitation, phenyl, naphthyl, anthracenyl, or phenanthrenyl. Aryl radicals may be unsubstituted, where all carbon atoms which are substitutable bear hydrogen atoms. Alternatively, they may be substituted at one, greater than one, or at all substitutable positions therein. Suitable exemplary substituents include, without limitation, alkyl radicals, such as alkyl radicals having 1 to 8 carbon atoms, which may be selected from methyl, ethyl, i-propyl or t-butyl, aryl radicals (such as C₆-aryl radicals, which may be substituted or unsubstituted), heteroaryl radicals (which may comprise at least one nitrogen atom, such as pyridyl radicals), alkenyl radicals (which may comprise one double bond and 1 to 8 carbon atoms), or groups with electron donating or electron accepting ability. Groups with electron donating ability are understood to mean groups which have a positive inductive (+I) and/or positive mesomeric (+M) effect, and groups with electron accepting ability are understood to mean groups which have a negative inductive (−I) and/or negative mesomeric (−M) effect. Suitable groups with donor or acceptor action are halogen radicals, such as F, Cl, Br, alkoxy radicals, aryloxy radicals, carbonyl radicals, ester radicals, amine radicals, amide radicals, CH₂F groups, CHF₂ groups, CF₃ groups, CN groups, thio groups, or SCN groups.

A “heteroaryl radical” or “heteroaryl group” is understood to mean radicals which differ from the aryl radicals described above in that at least one carbon atom in the structure making up the aryl radical is otherwise replaced by at least one heteroatom. Heteroatoms may have hydrogen substituents and/or any permissible substituents of organic compounds in order to satisfy the valences of the heteroatoms. Exemplary heteroatoms include N, O, and S. In most instances, one or two carbon atoms of the structure of the aryl radicals are replaced by heteroatoms. Exemplary heteroaryls include, without limitation, pyridyl, pyrimidyl, pyrazyl, triazyl, and five-membered heteroaromatics, such as pyrrole, furan, thiophene, pyrazole, imidazole, triazole, oxazole, thiazole. Heteroaryls may be substituted at none (unsubstituted), one, more than one, or at all substitutable positions. Suitable substituents are as defined above for the aryl radicals.

An “alkyl radical” or “alkyl group” is understood to mean a radical having 1 to 20 carbon atoms, 1 to 10 carbon atoms, or 1 to 8 carbon atoms. The alkyl radical may be branched or unbranched and the carbon chain may optionally be interrupted by one or more heteroatoms, such as N, O, or S. Heteroatoms may have hydrogen substituents and/or any permissible substituents of organic compounds in order to satisfy the valences of the heteroatoms. The alkyl radical may optionally be substituted by one or more of the substituents mentioned for the aryl radicals above. It is also possible that the alkyl radical contain one or more aryl groups thereon, where suitable aryl groups are described above. Exemplary alkyl radicals include, without limitation, methyl, ethyl, i-propyl, n-propyl, i-butyl, n-butyl, t-butyl, sec-butyl, i-pentyl, n-pentyl, sec-pentyl, neopentyl, n-hexyl, i-hexyl and sec-hexyl.

An “alkenyl radical” or “alkenyl group” is understood to mean a radical having 2 to 20 carbon atoms, 2 to 10 carbon atoms, or 2 to 8 carbon atoms, which may be optionally substituted and at least one carbon-carbon double bond.

An “alkynyl radical” or “alkynyl group” is understood to mean a radical having 2 to 20 carbon atoms, 2 to 10 carbon atoms, or 2 to 8 carbon atoms, which may be optionally substituted and at least one carbon-carbon triple bond.

A “cycloalkyl radical” or “cycloalkyl group” is understood to mean a cyclic radical having 3 to 20 carbon atoms, 3 to 10 carbon atoms, or 3 to 8 carbon atoms. The carbon chain of the cycloalkyl radical may optionally be interrupted by one or more heteroatoms, such as N, O, or S forming a heterocycloalkyl. Heteroatoms may have hydrogen substituents and/or any permissible substituents of organic compounds in order to satisfy the valences of the heteroatoms. The cycloalkyl radical may be unsubstituted or substituted, i.e. substituted by one or more of the substituents mentioned herein.

A “cycloalkenyl radical” or “alkenyl group” is understood to mean a cyclic radical having 4 to 20 carbon atoms, 4 to 10 carbon atoms, or 4 to 8 carbon atoms, which may be optionally substituted and at least one carbon-carbon double bond.

A “cycloalkynyl radical” or “cycloalkynyl group” is understood to mean a cyclic radical having 6 to 20 carbon atoms, 6 to 10 carbon atoms, or 6 to 8 carbon atoms, which may be optionally substituted and at least one carbon-carbon triple bond.

“Carbonyl group,” as used herein, is understood to mean moieties which can be represented by the general formula:

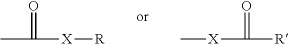

wherein X is a bond, or represents an oxygen or a sulfur, and R represents a hydrogen, a substituted or unsubstituted alkyl, substituted or unsubstituted alkenyl, substituted or unsubstituted alkynyl, substituted or unsubstituted cycloalkyl, substituted or unsubstituted aryl, or substituted or unsubstituted heteroaryl, —(CH₂)ₘ—R″; wherein R′ represents a hydrogen, substituted or unsubstituted alkyl, substituted or unsubstituted alkenyl, substituted or unsubstituted alkynyl, substituted or unsubstituted cycloalkyl, substituted or unsubstituted aryl, or substituted or unsubstituted heteroaryl or —(CH₂)ₘ—R″; wherein R″ represents a hydroxy group, substituted or unsubstituted carbonyl group, an aryl, a cycloalkyl, a heterocycle, or a polycycle; and m is zero or an integer ranging from 1 to 8. Where X is oxygen and R is defined as above, the moiety can be referred to as a “carboxyl group.” When X is oxygen and R is hydrogen, the formula represents a “carboxylic acid group.” Where X is oxygen and R′ is hydrogen, the formula represents a “formate group.” Where X is oxygen and R or R′ is not hydrogen, the formula represents an “ester group.” In general, where the oxygen atom of the above formula is replaced by a sulfur atom, the formula represents a “thiocarbonyl group.” Where X is sulfur and R or R′ is not hydrogen, the formula represents a “thioester group.” Where X is sulfur and R is hydrogen, the formula represents a “thiocarboxylic acid group.” Where X is sulfur and R′ is hydrogen, the formula represents a “thioformate group.” Where X is a bond and R is not hydrogen, the above formula represents a “ketone group.” Where X is a bond and R is hydrogen, the above formula represents an “aldehyde group.” The term “substituted carbonyl” refers to a carbonyl, as defined above, wherein one or more hydrogen atoms in R, R′ or a group to which the moiety is attached, are independently substituted with suitable substituents, as defined below.

An “amide group” or “amido” is understood to mean a moiety represented by the general formula:

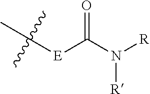

wherein, E is absent, or E is substituted or unsubstituted alkyl, substituted or unsubstituted alkenyl, substituted or unsubstituted alkynyl substituted or unsubstituted cycloalkyl, substituted or unsubstituted aryl, substituted or unsubstituted heteroaryl, wherein independently of E, R and R′ each independently represent a hydrogen, substituted or unsubstituted alkyl, substituted or unsubstituted alkenyl, substituted or unsubstituted alkynyl, substituted or unsubstituted carbonyl, substituted or unsubstituted cycloalkyl, substituted or unsubstituted aryl, or substituted or unsubstituted heteroaryl, —(CH₂)ₘ—R″′, or R and R′ taken together with the N atom to which they are attached complete a heterocycle having from 3 to 14 atoms in the ring structure; R′″ can represent a hydroxy group, substituted or unsubstituted carbonyl group, an aryl group, a cycloalkyl group, a heterocycle, or a polycycle; and m is zero or an integer ranging from 1 to 8. When E is oxygen, a “carbamate group” is formed. The carbamate cannot be attached to another chemical species, such as to form an oxygen-oxygen bond, or other unstable bonds, as understood by one of ordinary skill in the art.

The term “substituted,” as used herein, refers to all permissible substituents of the compounds or functional groups described above. Exemplary substituents include, but are not limited to, halogens, hydroxyl groups, or any other organic groupings containing any number of carbon atoms, preferably 1-14 carbon atoms, and optionally include one or more heteroatoms, such as oxygen, sulfur, or nitrogen grouping in linear, branched, or cyclic structural formats. Representative substituents can include alkyl, substituted alkyl (such as —CF₃ and —CD₃), alkenyl, substituted alkenyl, alkynyl, substituted alkynyl, phenyl, substituted phenyl, aryl, substituted aryl, heteroaryl, substituted heteroaryl, halo, hydroxyl, alkoxy, formyl, substituted alkoxy, phenoxy, substituted phenoxy, aroxy, substituted aroxy, thio (—SH), substituted thio, arylthio, substituted arylthio, cyano, isocyano, substituted isocyano, carbonyl, substituted carbonyl, carboxyl, substituted carboxyl, carboxylates, amino, substituted amino, amide, substituted amide, sulfonyl, substituted sulfonyl, sulfonic acid, phosphoryl, substituted phosphoryl, phosphonyl, substituted phosphonyl, polyaryl, substituted polyaryl, cyclic (such as C₃-C₂₀ cyclic), substituted cyclic (such as substituted C₃-C₂₀ cyclic), heterocyclic, substituted heterocyclic, deuterium, trihaloalkyl (trifluoromethyl), unsubstituted diarylamino, substituted diarylamino, unsubstituted dialkylamino, substituted dialkylamino, azo, carbonate ester, nitro, nitroso, phosphino, pyridyl, NRR′, SR, C(O)R, COOR, C(O)NR, SOR, and SOR groups, wherein R and R′ are independently selected from hydrogen atom, deuterium atom, or any of the substituents named above.

Numerical ranges disclosed in the present application include, but are not limited to, ranges of carbon atoms, ranges of temperatures, ranges of concentrations, ranges of times, amongst other ranges disclosed below. The disclosed ranges, disclose individually each possible number that such a range could reasonably encompass, as well as any sub-ranges and combinations of sub-ranges encompassed therein. For example, disclosure of a range of carbon atoms is intended to disclose individually every possible value that such a range could encompass, consistent with the disclosure herein. For example, a carbon range of 1 to 10 carbons also discloses each number of carbons within the range individually (1, 2, 3, 4, 5, 6, 7, 8, 9, 10 carbons), as well as any sub-range contained therein (2 to 4 carbons or 5 to 9 carbons).

“Electron withdrawing group,” as used herein refers to a functional group or

atom within a molecule that has a tendency to withdraw electron density from the rest of the molecule typically due to its electronegative nature or its ability to attract electrons through resonance or inductive effects. Electron withdrawal can occur through different mechanisms, such as: (1) Electronegativity; (2) Resonance Effects; (3) Inductive Effects. Non-limiting exemplary functional groups include carbonyl groups (C═O), nitro groups (—NO₂), and halogens (e.g., —Cl, —Br, —I).

Use of the term “about” is intended to describe values either above or below the stated value, which the term “about” modifies, in a range of approx. +/−10%; in other instances the values may range in value either above or below the stated value in a range of approx. +/−5%. When the term “about” is used before a range of numbers (i.e., about 1-5) or before a series of numbers (i.e., about 1, 2, 3, 4, etc.) it is intended to modify both ends of the range of numbers and/or each of the numbers recited in the entire series, unless specified otherwise.

### II. Heterogeneous Catalysts

Heterogeneous catalysts which can be used in chemical reactions, such as [2+2]cycloadditions are described herein. In one instance, a non-limiting example a heterogenous catalyst includes:
a metal-organic framework formed of a plurality of inorganic nodes and a plurality of organic linkers wherein at least one organic linker of the plurality comprises a phenanthroline moiety;
wherein at least one copper (I) ion is ligated to the phenanthroline moiety and ligated to a phosphine ligand forming a catalyst complex within the metal-organic framework.

In some instances, the phosphine ligand can have a chemical structure according to the genus formula below:

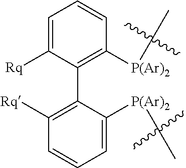

wherein Rq and Rq′ are each independently selected from hydrogen, alkyl (such as n-Bu), alkoxy (such as methoxy), or aryl (such as phenyl) groups; and
wherein each Ar is an aryl group, which can be independently selected from, for example, a phenyl or aromatic group. In certain instances, the aromatic group has a chemical structure as follows:

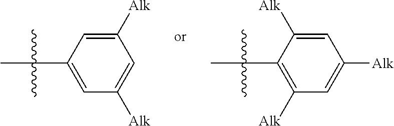

wherein each Alk is independently an alkyl group (such as methyl or t-butyl). The chemical moieties (i.e., alkyl, alkoxy, aryl) named above may be substituted or unsubstituted. In some instances, the phosphine ligand is 2,2′-bis(diphenylphosphino)-1,1′-binapthyl (binap).

In some instances, the plurality of inorganic nodes of the heterogenous catalyst include or are [Zr₆] inorganic nodes. In some instances, the plurality of organic linkers of the heterogenous catalyst include quaterphenyl dicarboxylate organic linkers. In certain instances, the quaterphenyl dicarboxylate organic linkers include or are tetramethyl substituted quaterphenyl dicarboxylate organic linkers. In certain instances, the tetramethyl substituted quaterphenyl can be 2′,2″,5′,5″-tetramethyl-[1,1′:4′,1″:4″,1″′-quaterphenyl]-4,4″′-dicarboxylic acid.

The heterogenous catalyst includes at least one organic linker which has a phenanthroline moiety, which can be derived from 4,4′-(1,10-phenanthroline-3,8-diyl)dibenzoic acid. “Derived,” as used herein refers to the molecule which was used to synthesize the metal-organic framework (MOF) and is understood to become part of the MOF structure.

In preferred instances, the metal-organic framework is a Universitetet Oslo-69 metal-organic framework. In preferred instances, the metal-organic framework is microporous. In some instances, the metal-organic framework has a Brunauer-Emmett-Teller (BET) surface area of at least about 1,000 m² g⁻¹, 1,250 m² g⁻¹, 1,500 m² g⁻¹, 1,750 m² g⁻¹, or 2,000 m² g⁻¹; or the metal-organic framework has a Brunauer-Emmett-Teller (BET) surface area in a range from between about 1,000 and 2,000 m² g⁻¹, as well as sub-ranges or individual values contained within the aforementioned range. In some instances, the metal-organic framework has pore width distribution centered at about 1.3, 1.4, 1.5, 1.6, or 1.7 nm; or the metal-organic framework has pore width distribution centered in a range from between about 1.2 to 1.8 nm, as well as sub-ranges or individual values contained within the aforementioned range.

In certain instances, the catalyst complex within the metal-organic framework includes a chemical structure as follows:

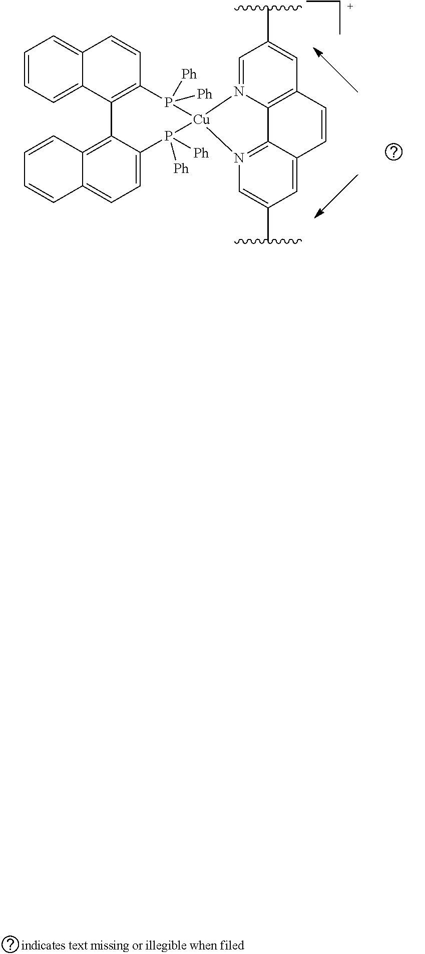

where the wavy bonds extending from the phenanthroline moiety are understood by the skilled artisan to be part of an organic linker containing the moiety within the metal-organic framework.

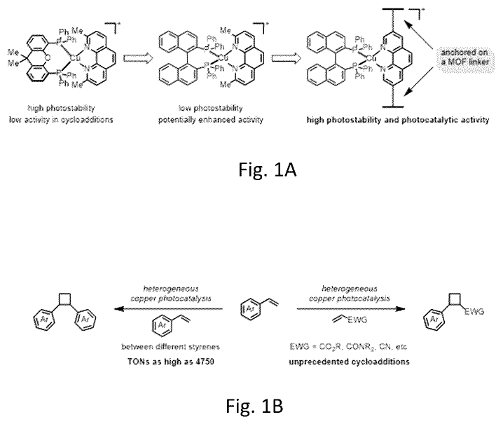

The heterogeneous catalysts (i.e., heteroleptic copper(I) complexes with bidentate nitrogen and phosphorus donor ligands) function as photoredox catalysts and (high) triplet photosensitizers, which can be used for a wide range of light-mediated organic transformations. It is believed that anchoring the catalyst complex within the MOF framework improves the photostability of such copper-binap complexes. Heterogenizing the labile transition metal species onto such suitable framework matrices can provide for catalyst-site isolation(Rogge, S. M. et al. Metal-organic and covalent organic frameworks as single-site catalysts. Chem. Soc. Rev. 46, 3134-3184 (2017); Wei, Y.-S., Zhang, M., Zou, R. & Xu, Q. Metal-organic framework-based catalysts with single metal sites. Chem. Rev. 120, 12089-12174 (2020); Chen, W. et al. Site-isolated azobenzene-containing metal-organic framework for cyclopalladated catalyzed Suzuki-Miyuara coupling in flow. ACS Appl. Mater. Interfaces 13, 51849-51854 (2021)). For example, by incorporating binap-ligated heteroleptic copper(I) species onto the organic linker of a structurally stable MOF support it was found that it was possible to prevent the photoinduced decomposition of copper triplet photosensitizers and extend their excited-state lifetimes due to MOF-enabled confinement effects(Liu, J. et al. MOF-enabled confinement and related effects for chemical catalyst presentation and utilization. Chem. Soc. Rev. 51, 1045-1097 (2022)) (see FIG. 1a). Such heterogeneous catalysts can be used in visible-light-driven [2+2]cycloadditions through energy transfer to appropriate reactants. The heterogeneous catalysts described can, for example, efficiently promote iminyl radical-mediated reactions(Ma, B. et al. Metal-organic framework supported copper photoredox catalysts for iminyl radical-mediated reactions. Angew. Chem. Int. Ed. 62, e202300233 (2023)).

The heterogeneous catalysts can provide high turnover numbers (TON) in a range of about 50 to 5000 turnovers, as well as sub-ranges or individual values contained within the aforementioned range.

The heterogeneous catalysts typically have relatively long photoexcited-state lifetimes. For instance, the heterogeneous catalysts can have an excited-state lifetime at room temperature of at least about 3, 3.5, 4, 4.5, or 5 μs; or an excited-state lifetime at room temperature in a range of between about 3 to 5 μs, as well as sub-ranges or individual values contained within the aforementioned range.

### III. Methods of Making Heterogeneous Catalysts

The heterogeneous catalysts described can be synthesized by various methods. In one instance, a non-limiting method of synthesizing a heterogenous catalyst includes the steps of:
(i) reacting a plurality of organic linkers, wherein at least one organic linker of the plurality includes a phenanthroline moiety, with an inorganic salt to form a metal-organic framework comprising a plurality of inorganic nodes;
(ii) metalating the metal-organic framework by mixing a metal complex comprising at least one copper (I) ion ligated to a phosphine ligand with the metal-organic framework;
where the metalating step includes the least one copper (I) ion becoming ligated to the phenanthroline moiety to form a catalyst complex within the metal-organic framework.

In another non-limiting instance, a method of synthesizing a heterogenous catalyst includes the steps of:
(i′) metalating a metal-organic framework by mixing a metal complex comprising at least one copper (I) ion ligated to a phosphine ligand with the metal-organic framework;
where the metal-organic framework is formed of a plurality of inorganic nodes and a plurality of organic linkers wherein at least one organic linker of the plurality includes a phenanthroline moiety;
where the metalating step comprises the least one copper (I) ion becoming ligated to the phenanthroline moiety to form a catalyst complex within the metal-organic framework.

In some instances, the phosphine ligand can have a chemical structure according to:

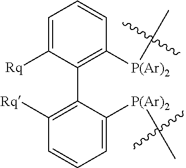

where Rq and Rq′ are each independently selected from hydrogen, alkyl (such as n-Bu), alkoxy (such as methoxy), or aryl (such as phenyl) groups; and
where each Ar is an aryl group, which can be independently selected from, for example, a phenyl or aromatic group. In certain instances, the aromatic group has a chemical structure as follows:

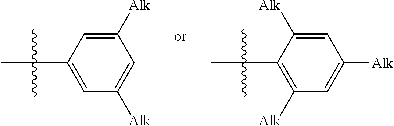

where each Alk is independently an alkyl group (such as methyl or t-butyl). The chemical moieties (i.e., alkyl, alkoxy, aryl) named above may be substituted or unsubstituted. In some instances, the phosphine ligand is 2,2′-bis(diphenylphosphino)-1,1′-binapthyl (binap).

In certain instances, the inorganic salt used in step (i) of the first method is a zirconium salt, such as ZrCl₄. Other inorganic salts are known to the person of ordinary skill in the art. In some instances, the plurality of inorganic nodes of the metal-organic framework include or are [Zr₆] inorganic nodes.

In some instances of the methods, the plurality of organic linkers of the heterogenous catalyst formed include quaterphenyl dicarboxylate organic linkers. In certain instances, the quaterphenyl dicarboxylate organic linkers include or are tetramethyl substituted quaterphenyl dicarboxylate organic linkers. In certain instances, the tetramethyl substituted quaterphenyl can be 2′,2″,5′,5″-tetramethyl-[1,1′:4′,1″:4″,1″′-quaterphenyl]-4,4″′-dicarboxylic acid.

The heterogenous catalyst formed according to the methods above includes at least one organic linker which has a phenanthroline moiety, which can be derived from 4,4′-(1,10-phenanthroline-3,8-diyl)dibenzoic acid. “Derived,” as used herein refers to the molecule which was used to synthesize the metal-organic framework (MOF) and is understood to become part of the MOF structure.

In preferred instances, the metal-organic framework is a Universitetet Oslo-69 metal-organic framework. In preferred instances, the metal-organic framework is microporous.

In certain instances of the first method, step (i) proceeds in an organic solvent comprising a Lewis acid and heating at a temperature of at least about 100° C.; wherein the organic solvent is optionally dimethylformamide and the Lewis acid is optionally trifluoroacetic acid.

Typically, the metalating step of the methods proceeds in an organic solvent at room temperature under an inert atmosphere; optionally where the organic solvent is dichloromethane and optionally wherein the inert atmosphere is a nitrogen atmosphere.

In certain instances of the methods, the metal complex used in the metaling step is:

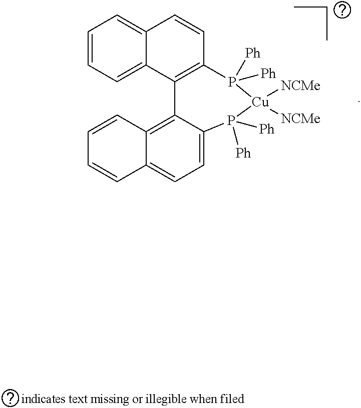

Other metal complexes may be used to achieve the described heterogeneous catalysts.

In preferred instances, the methods produce a heterogeneous catalyst where the catalyst complex attached to the metal-organic framework has a chemical structure as follows:

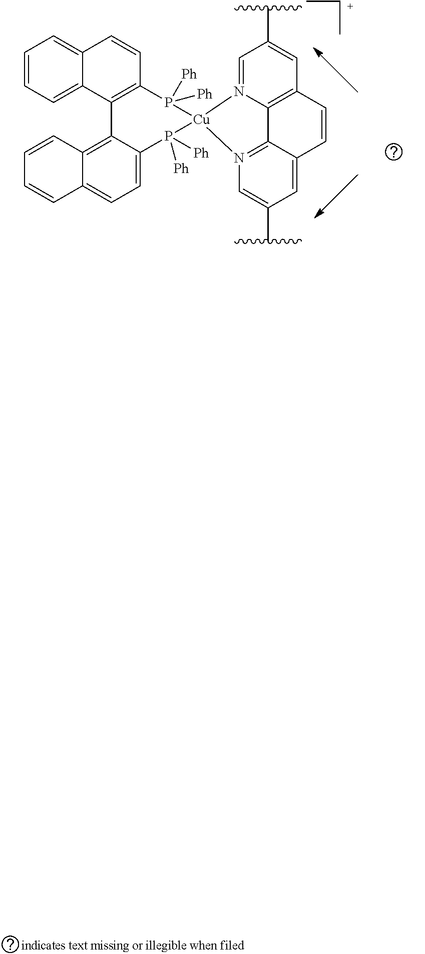

where the wavy bonds extending from the phenanthroline moiety are understood by the skilled artisan to be part of an organic linker containing the moiety within the metal-organic framework.

The skilled person understands that the heterogeneous catalysts described above and synthesized by the exemplary methods may have one or more chiral centers and thus exist as one or more stereoisomers. Such stereoisomers can exist as a single enantiomer, a mixture of diastereomers or a racemic mixture are encompassed by the present disclosure. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

For the methods described, the synthetic conditions (choice of solvent(s), temperatures, atmospheres, work-up conditions, purification conditions, etc.) can be selected or modified, as appropriate, by the person of ordinary skill in the art of synthetic chemistry.

The formed heterogeneous catalysts may be characterized using suitable techniques known to the person of ordinary skill. Without limitation, the heterogeneous catalysts synthesized according to the methods described can be analyzed via ¹H and ¹³C NMR, ICP-AES, BET analysis, powder XRD, EDS, XPS, EXAFS, TGA, and combinations thereof.

### IV. Methods of Using Heterogeneous Catalysts

The heterogeneous catalysts described can be used in large-scale synthesis and demonstrate catalyst recyclability, as well as facilitate the separation of catalyst and product. Moreover, the heterogeneous catalysts can be employed to achieve organic transformations that are not known to be accomplished in previously established photocatalytic systems, such as intermolecular [2+2]cycloadditions of styrenes with electron-deficient olefins.

At present, the cost for producing the heterogeneous copper photocatalysts is about 30 to 50 times lower than the cost of producing commercialized noble-metal photocatalysts. In general, homogeneous noble-metal photocatalysts cannot be recycled after a photocatalytic reaction, whereas the heterogeneous catalysts described can be recovered from the reaction mixture without significant loss of reactivity. This is in contrast to the tedious separation processes used to remove homogeneous photocatalysts from solution, separation in heterogeneous photocatalysis can be achieved through filtration methods known to the skilled person.

In one non-limiting instance, the heterogeneous catalysts can be used to perform a [2+2]cycloaddition, in a method including the steps of:
(a) forming a mixture of a first compound and a second compound in an organic solvent including one or more heterogenous catalysts as described herein;
wherein each of the first and the second compounds includes a carbon-carbon double bond capable of undergoing a [2+2]cycloaddition; and
(b) exposing the mixture to visible light irradiation to photoexcite the heterogenous catalyst wherein an energy transfer from the photoexcited heterogenous catalyst induces a [2+2]cycloaddition between the carbon-carbon double bonds of the first and the second compounds.

As discussed in the examples, a cyclobutane is formed from the [2+2]cycloaddition between the carbon-carbon double bonds of the first and the second compounds.

In some instances of the method, at least one of the first and the second compounds is or includes an electron deficient alkene therein. In other instances, at least one of the first and the second compounds is a styrene compound or a vinylnapthalene compound, which may be substituted. In still other instances, at least one of the first and/or the second compounds comprises an U,D-unsaturated carbonyl. In certain instances, at least one of the first and/or the second compounds has a chemical structure as follows:

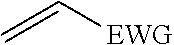

wherein EWG is an electron withdrawing group. Without limitation, the electron withdrawing group can be selected from —C(O)OR; —C(O)R′; —C(O)NR₁R₂, or —CN; where R, R′, R₁, and R₂ are each independently selected from hydrogen; halogen group (i.e., —F, —Cl, —Br, —I); a C₁-C₅ alkyl group (linear or branched), such as a methyl, ethyl, propyl, butyl, or pentyl group; alkenyl group; alkynyl group; cycloalkyl group; cycloalkenyl group; cycloalkynyl group; a hydroxyl group; an aryl group (i.e., a phenyl group); a heteroaryl group; a benzyl group; an acyl group; an ester group; a carbonyl group; a carboxylate group; an amino group (primary, secondary, or tertiary); an amide group; and a nitro group. In other instances, at least one of the first and/or the second compounds can have a chemical structure according to any one of Formulae (I)-(IV):

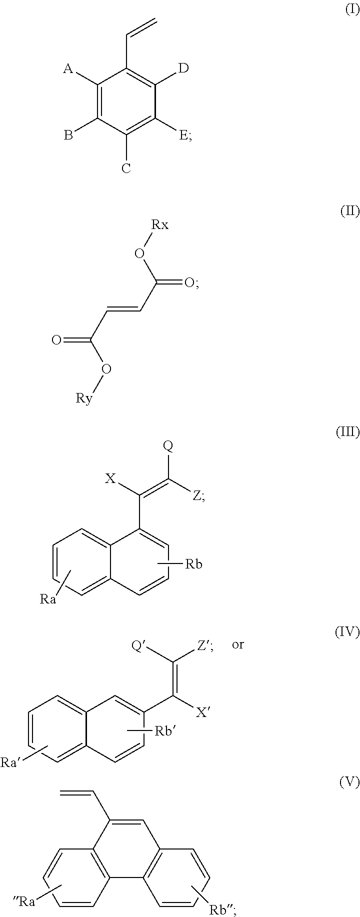

where A, B, C, D, and E are each independently selected from a hydrogen, halogen group (i.e., —F, —Cl, —Br, —I); a C₂-C₅ alkyl group (linear or branched), such as a methyl, ethyl, propyl, butyl, or pentyl group; alkenyl group; alkynyl group; cycloalkyl group; cycloalkenyl group; cycloalkynyl group; a hydroxyl group; an alkoxy group, such as methoxy, ethoxy, propoxy, or butoxy; an aryl group (i.e., a phenyl group); a heteroaryl group; a benzyl group; an acyl group; an ester group; a carbonyl group; a carboxylate group; an amino group (primary, secondary, or tertiary); an amide group; and a nitro group; optionally wherein A and B; B and C; C and E; and E and D may in combination form a cycloalkyl group, heterocycloalkyl group, aryl group, or heteroaryl group;
where Rx and Ry are each independently selected from hydrogen or an alkyl group (such as a methyl group);
where Ra, Ra′, Ra″, Rb, Rb′, and Rb″ are each independently selected from hydrogen, hydrogen, halogen group (i.e., —F, —Cl, —Br, —I); a C₂-C₅ alkyl group (linear or branched), such as a methyl, ethyl, propyl, butyl, or pentyl group; alkenyl group; alkynyl group; cycloalkyl group; cycloalkenyl group; cycloalkynyl group; a hydroxyl group; an alkoxy group, such as methoxy, ethoxy, propoxy, or butoxy; an aryl group (i.e., a phenyl group); a heteroaryl group; a benzyl group; an acyl group; an ester group; a carbonyl group; a carboxylate group; an amino group (primary, secondary, or tertiary); an amide group; and a nitro group;
where Q, Q′, X, X′, Z, and Z′ are each independently selected from hydrogen and alkyl groups; optionally wherein Q and Z and Q′ and Z′ may in combination form a cycloalkyl or heterocycloalkyl group;
where optionally a carbon of the aromatic rings of Formulae (I), (III), (IV), or (V) is substituted by a nitrogen.

Various combinations of first and the second compounds including carbon-carbon double bonds capable of undergoing a [2+2]cycloaddition, as named above, are possible.

In some instances of the method, the heterogenous catalyst can be present in the mixture in a concentration of between about 0.01 to 2 mol % of the total mixture.

In some cases, the mixture further includes 4-dimethylaminopyridine.

In some instances, wherein the visible light irradiation includes blue light, where the blue light can be at or include a wavelength of 440 nm.

In some instances, the organic solvent is or includes 1,2-dichloroethane. Other suitable organic solvents may be used.

Step (b) typically proceeds under an inert atmosphere, where the inert atmosphere can be a nitrogen atmosphere. Other suitable inert atmospheres can be used (i.e., argon, etc.).

For the method described, a further step of recovering the heterogeneous catalyst from the mixture following step (b) can be carried out. As mentioned above, such recovery can be accomplished using art known filtration techniques. The recovered heterogeneous catalyst can be reused to perform another [2+2]cycloaddition at least one, two, three, four, five, six, or more times without loss of reactivity or significant loss of reactivity. “Significant loss,” as used herein, refers to a loss of reactivity of no more than about 10, 20, 30, 40, or 50% of the reactivity, as compared to the catalyst when first used.

The heterogeneous catalysts can provide high turnover numbers (TON) in catalyzing cycloaddition products, where the TON can be in a range of about 50 to 5000 turnovers, as well as sub-ranges or individual values contained within the aforementioned range.

The skilled person understands that the [2+2]cycloaddition products described above and synthesized using the heterogeneous catalysts may have one or more chiral centers and thus exist as one or more stereoisomers. Such stereoisomers can exist as a single enantiomer, a mixture of diastereomers or a racemic mixture are encompassed by the present disclosure. As used herein, the term “stereoisomers” refers to compounds made up of the same atoms having the same bond order but having different three-dimensional arrangements of atoms which are not interchangeable. The three-dimensional structures are called configurations. As used herein, the term “enantiomers” refers to two stereoisomers which are non-superimposable mirror images of one another. As used herein, the term “optical isomer” is equivalent to the term “enantiomer”. As used herein the term “diastereomer” refers to two stereoisomers which are not mirror images but also not superimposable. The terms “racemate”, “racemic mixture” or “racemic modification” refer to a mixture of equal parts of enantiomers. The term “chiral center” refers to a carbon atom to which four different groups are attached. Choice of the appropriate chiral column, eluent, and conditions necessary to effect separation of the pair of enantiomers is well known to one of ordinary skill in the art using standard techniques (see e.g. Jacques, J. et al., “Enantiomers, Racemates, and Resolutions”, John Wiley and Sons, Inc. 1981).

For the methods described above, the synthetic conditions (choice of solvent(s), temperatures, atmospheres, work-up conditions, purification conditions, etc.) can be selected or modified, as appropriate, by the person of ordinary skill in the art of synthetic chemistry.

In some instances, the use of the heterogeneous catalysts can provide [2+2]cycloaddition products with high diastereoselectivity. In some instances, the diastereoselectivity is defined as a diastereomeric ration (d.r.), which can be in a range from >1:1 to about 10:1 between to diastereomer products produced, as well as sub-ranges or individual values contained within the aforementioned range.

The heterogeneous catalysts and methods described herein are further illustrated in the following examples, which are provided by way of illustration and are not intended to be limiting. It will be appreciated that variations in proportions and alternatives in elements of the components shown will be apparent to those skilled in the art and are within the scope of disclosed forms. Theoretical aspects are presented with the understanding that Applicant does not seek to be bound by the theory presented.

### EXAMPLES

### Example 1: Heterogeneous Copper-Based Photocalyst for [2+2] Cycloadditions

### Materials and Methods:

### General Considerations:

PXRD patterns were recorded on a Rigaku Ultima IV X-ray diffractometer (CuKα, λ=1.54184 Å), operating at 40 kV and 30 mA. The measurement parameters included a scan speed of 100 min⁻¹, a step size of 0.05° and a scan range of 2θ from 5° to 40°.

Scanning electron microscopy (SEM) and energy dispersive spectroscopy (EDS) mapping were collected on an on VEGA3 TESCAN (Japan). The X-ray photoelectron spectroscopy (XPS) surface investigation was carried out on a Thermo ESCALAB 250Xi system and the spectra were analyzed using the Thermo Scientific Avantage Data System software. Transmission electron microscopy (TEM) and energy dispersive X-ray (EDX) analyses were performed by FEI-Talos F200S.

Inductively couple plasma—atomic emission spectroscopy (ICP-AES) measurements were conducted on a Thermo Scientific iCAP 7000 ICP-AES analyzer.

Thermogravimetric analyses (TGA) was performed on a Mettler-Toledo (TGA/DSC1) thermal analyzer (Q50 V20.6). Approximately 5 mg of dried samples were measured under a N₂ flow rate of 20 ml min⁻¹ with a heating rate of 10° C. min⁻¹ from 40 to 800° C.

X-ray diffraction data of single crystals were collected at 173 K on a XtaLab PRO MM007HF DW diffractometer system equipped with a MicroMax-007DW MicroFocus X-ray generator and a Pilatus 200 K silicon diarray detector (Rigaku, Japan, CuKα, λ=1.54184 Å). Structures were solved by direct methods using the SHELXT program and refined by full-matrix least-squares on F² with anisotropic displacement parameters for all non-hydrogen atoms using the SHELXL program.

X-ray absorption fine structure (XAFS) measurements were performed on beamline 01B1 and 14B2 at SPring-8 of the Japan Synchrotron Radiation Research Institute (8 GeV, 99.5 mA). Spectra of Cu K-edge XAFS (20 min per spectrum) were recorded in a fluorescence mode using an ionization chamber with Si(111) under atmospheric conditions and detuned to reduce the contribution of higher-order harmonics below the level of noise. A metallic foil standard was used as a reference for energy calibration and was measured simultaneously with the experimental samples. Ion chambers filled with N₂ (100%) and N₂ (70%)/Ar (30%) were used for the I₀ and I_{f} detectors. XAFS results were processed according to the standard procedures using the ATHENA module in the IFEFFIT software packages. The k³-weighted EXAFS spectra were obtained by subtracting the post-edge background from the overall absorption and then normalising with respect to the edge-jump step. Subsequently, k³-weighted x(k) data of Cu K-edge were Fourier transformed to R-space using Hanning windows (dk=1.0 Å⁻¹) to separate the EXAFS contributions from different coordination shells. To obtain the quantitative structural parameters around central atoms, least-squares curve parameter fitting was performed using the ARTEMIS module in the IFEFFIT software packages. Refinement was performed by optimizing an amplitude factor S₀² and energy shift ΔE₀ which are common to all paths, in addition to parameters for bond length (ΔR) and Debye-Waller factor (σ₂). The fitting models for the Cu sites in UiO-69-phen(binap)Cu and UiO-69-phen(xantphos)Cu were based on the crystal structures of Cu-1 (CCDC: 2240330) and Cu-3 (CCDC: 2244329), respectively.

Solid-state UV-Vis absorption spectra of the framework materials were recorded at room temperature on a UV-2550 spectrophotometer (SHIMADZU, Japan).The steady-state emission spectra were measured on a Horiba Fluorog-3 spectrophotometer. The lifetime measurements were performed on a Quanta Ray GCR 150-10 PULSED Nd:YAG laser system.

Brunauer-Emmett-Teller (BET) surface area analysis was performed on a micromeritics surface area analyzer (ASAP 2420) at 77 K.

Cyclic voltammograms were performed on a CHI760E electrochemistry workstation. Regular 3-electrode systems were used. Measurements were recorded in a DMF solution of (Bu₄N)(ClO₄) (0.1 M) at a scan rate of 100 mV s⁻¹ under the protection of N₂ using a glassy carbon disk (d=0.3 cm) as a working electrode and a platinum plate (1 cm×1 cm) as a counter electrode. An Ag/AgCl (3 M KCl) electrode was used as a reference electrode in all the experiments, and its potential (−0.53 V vs. Fc/Fc) was calibrated with the ferrocenium/ferrocene (Fc⁺/Fc) redox couple.

¹H and ¹³C NMR spectra were recorded on a Bruker 500 or Bruker 400 spectrometer in CDCl₃, DMSO-d₆ or a mixed deuterated solvent.

High-resolution electron ionization (EI) mass spectra were recorded on a Thermo Scientific DFS Magnetic Sector GC-HRMS system. High-resolution electrospray ionization mass spectrometry (ESI-MS) measurements were performed on a Bruker impact II high-resolution LC-QTOF mass spectrometer. Accurate masses from high-resolution mass spectra were reported for the molecular ion [M]⁺ or [M+H]⁺.

### Synthesis of Catalyst Support UiO-69-phen

ZrCl₄ (179 mg, 0.77 mmol), 4,4′-(1,10-phenanthroline-3,8-diyl)dibenzoic acid (L2) (50 mg, 0.12 mmol), 2′,2″,5′,5″-tetramethyl-[1,1′:4′,1″:4″,1′″-quaterphenyl]-4,4″′-dicarboxylic acid (L3) (270 mg, 0.60 mmol), and trifluoroacetic acid (0.4 ml) were ultrasonically dispersed in N,N-dimethylformamide (DMF) (34 ml). The resulting solution was heated to 100° C. in a 50-ml glass tube for 72 hours. After cooling to room temperature, the supernatant was decanted and the solid was washed five times with DMF and acetone. UiO-69-phen was then collected by filtration and dried in air to give a white solid (389 mg, 60% yield based on linkers). The product (2 mg) was digested in a mixture of a saturated K₃PO₄/D₂O solution and DMSO-d₆ (0.6 ml, 1:2 v/v) and subjected to ¹H NMR analysis. The ratio of L2 and L3 was found to be 1:5. The chemical formula of UiO-69-phen was determined to be Zr₆O₄(OH)₄(L2)(L3)₅·4.0DMF based on NMR digestion data and TGA.

### Synthesis of Catalyst UiO-69-phenCu

UiO-69-phen (30.0 mg, 5.6 μmol based on L2) was immersed in a 2-ml dichloromethane solution of [Cu(MeCN)₄](PF₆) (10.4 mg, 28 μmol) and stirred under N₂ atmosphere at 60° C. for 4 hours. After cooling to room temperature, the supernatant was decanted and the solid was washed five times with DMF and acetone. UiO-69-phenCu was then collected by filtration and dried in air to give a green solid (22.7 mg, 93% yield). ICP-AES analysis indicated a Zr/Cu ratio of 6:1. The chemical formula of UiO-69-phenCu was determined to be {Zr₆O₄(OH)₄(L2)(L3)₅[Cu(CH₃CN)₂PF₆]}·4.4DMF based on ICP-AES and TGA.

### Synthesis of Catalyst UiO-69-phen(xantphos)Cu

UiO-69-phen (30.0 mg, 5.6 μmol based on L2) was immersed in a 2-ml dichloromethane solution of [Cu(xantphos)(MeCN)₂](PF₆) (24.3 mg, 28 μmol) and stirred under N₂ atmosphere at room temperature for 9 hours. After post-synthetic metalation, the supernatant was decanted and the solid was washed five times with DMF and acetone. UiO-69-phen(xantphos)Cu was then collected by filtration and dried in air to give a yellow solid (30.9 mg, 91% yield). ICP-AES analysis indicated a Zr/Cu ratio of 6:1. The product (2 mg) was digested in a solution of D₃PO₄/D₂O/DMSO-d₆ (1:1:5 v/v/v) and subjected to ¹H NMR analysis. The ratio of Cu-4 and L3 was found to be 1:5. The chemical formula of UiO-69-phen(xantphos)Cu was determined to be {Zr₆O₄(OH)₄(L2)(L3)₅[Cu(xantphos)PF₆]}·4.9DMF based on NMR digestion data and TGA.

### Synthesis of Catalyst UiO-69-phen(binap)Cu

UiO-69-phen (300 mg, 56 μmol based on L2) was immersed in a 20-ml dichloromethane solution of [Cu(binap)(MeCN)₂](PF₆) (256 mg, 0.28 mmol) and stirred under N₂ atmosphere at room temperature for 9 hours. After post-synthetic metalation, the supernatant was decanted and the solid was washed five times with DMF and acetone. UiO-69-phen(binap)Cu was then collected by filtration and dried in air to give a yellow solid (312 mg, 94% yield). ICP-AES analysis indicated a Zr/Cu ratio of 6:1. The product (2 mg) was digested in a solution of D₃PO₄/D₂O/DMSO-d₆ (1:1:5 v/v/v) and subjected to ¹H NMR analysis. The ratio of Cu-2 and L3 was found to be 1:5. The chemical formula of UiO-69-phen(binap)Cu was determined to be {Zr₆O₄(OH)₄(L2)(L3)₅[Cu(binap)PF₆]}·6.0DMF based on NMR digestion data and TGA.

General Procedure for the Photoinduced Crossed [2+2] Cycloaddition of Styrenes with Electron-Deficient Alkenes:

To a 25-ml flame-dried Schlenk tube cooled under N₂ atmosphere were added UiO-69-phen(binap)Cu (2.4 mg, 0.4 μmol based on Cu, 0.2 mol %), 4-dimethylaminopyridine (29 mg, 0.24 mmol, 1.2 equiv.), 1,2-dichloroethane (1.0 ml), styrene (0.2 mmol, 1.0 equiv.), and electron-deficient alkene (0.6 or 1.0 mmol, 3.0 or 5.0 equiv.) sequentially. After three freeze-pump-thaw cycles, the reaction mixture was irradiated by three 40-watt Kessil PR160L-440 blue-LED lamps at room temperature (with three fans) for 48 hours. After irradiation, ethyl acetate (10 ml) was added into the crude mixture; the resulting solution was filtered through a pad of silica gel and washed four times with ethyl acetate (4×10 ml). The combined filtrates were concentrated under vacuum and the diastereomeric ratio was determined by ¹H NMR analysis. The residue was purified by flash column chromatography on silica gel to afford the corresponding product.

### General Procedure for the Photoinduced Crossed [2+2] Cycloaddition Between Different Styrenes:

To a 25-ml flame-dried Schlenk tube cooled under N₂ atmosphere were added UiO-69-phen(binap)Cu (2.4 mg, 0.4 μmol based on Cu, 0.02 mol %), 4-dimethylaminopyridine (293 mg, 2.4 mmol, 1.2 equiv.), 1,2-dichloroethane (1.0 ml), styrene (2.0 mmol, 1.0 equiv.), and the other coupling styrene (10 mmol, 5.0 equiv.) sequentially. After three freeze-pump-thaw cycles, the reaction mixture was irradiated by three 40-watt Kessil PR160L-440 blue-LED lamps at room temperature (with three fans) for 48 hours. After irradiation, ethyl acetate (10 ml) was added into the crude mixture; the resulting solution was filtered through a pad of silica gel and washed four times with ethyl acetate (4×10 ml). The combined filtrates were concentrated under vacuum and the diastereomeric ratio was determined by ¹H NMR analysis. The residue was purified by flash column chromatography on silica gel to afford the corresponding product.

General Procedure for the Photoinduced Crossed [2+2] Cycloaddition of Exocyclic Arylidene Azetidines, Thietanes, and Oxetanes with Electron-Deficient Alkenes and Styrenes:

To a 25-ml flame-dried Schlenk tube cooled under N₂ atmosphere were added UiO-69-phen(binap)Cu (2.4 mg, 0.4 μmol based on Cu, 0.2 mol %), 4-dimethylaminopyridine (29 mg, 0.24 mmol, 1.2 equiv.), 1,2-dichloroethane (1.0 ml), exocyclic arylidene azetidine, thietane, or oxetane (0.2 mmol, 1.0 equiv.), and electron-deficient alkene or styrene (0.6 mmol, 3.0 equiv.) sequentially. After three freeze-pump-thaw cycles, the reaction mixture was irradiated by three 40-watt Kessil PR160L-440 blue-LED lamps at room temperature (with three fans) for 48 hours. After irradiation, ethyl acetate (10 ml) was added into the crude mixture; the resulting solution was filtered through a pad of silica gel and washed four times with ethyl acetate (4×10 ml). The combined filtrates were concentrated under vacuum and the diastereomeric ratio was determined by ¹H NMR analysis. The residue was purified by flash column chromatography on silica gel to afford the corresponding product.

### Gram-Scale Reaction:

To a 100-ml flame-dried Schlenk tube cooled under N₂ atmosphere were added UiO-69-phen(binap)Cu (89 mg, 15 μmol based on Cu, 0.15 mol %), 4-dimethylaminopyridine (1.46 g, 12 mmol, 1.2 equiv.), 1,2-dichloroethane (50 ml), 1-vinylnaphthalene (1.54 g, 10 mmol, 1.0 equiv.), methyl acrylate (4.5 ml, 50 mmol, 5.0 equiv.) sequentially. After three freeze-pump-thaw cycles, the reaction mixture was irradiated by three 40-watt Kessil PR160L-440 blue-LED lamps at room temperature (with three fans) for 48 hours. After irradiation, ethyl acetate (100 ml) was added into the crude mixture; the resulting solution was filtered through a pad of silica gel and washed four times with ethyl acetate (4×50 ml). The combined filtrates were concentrated under vacuum and the diastereomeric ratio was determined by ¹H NMR analysis. The residue was purified by flash column chromatography on silica gel to afford 1 (1.68 g, 70% yield) as a colorless oil.

### Results:

### Synthesis and Characterization of MOF-Supported Copper Photocatalysts:

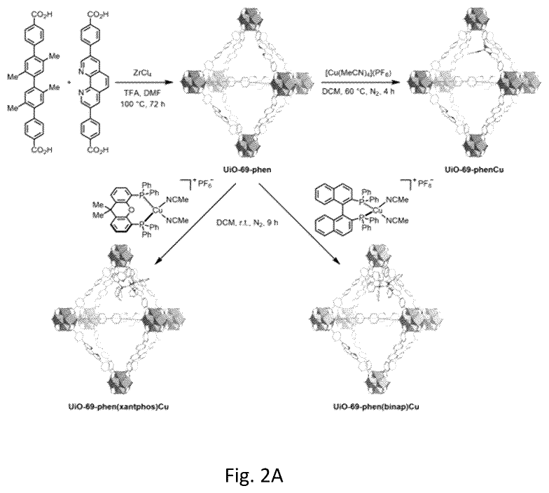

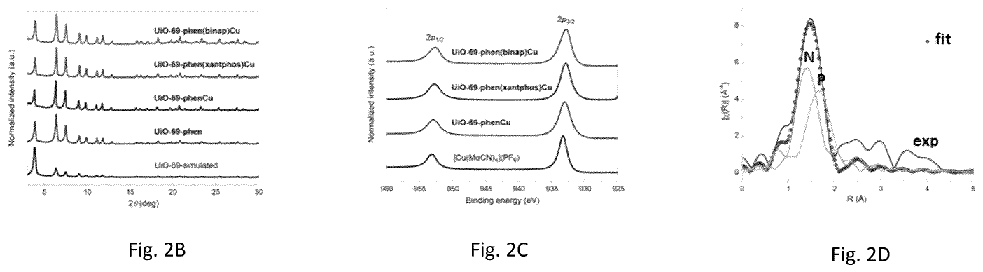

To accommodate a sterically hindered heteroleptic copper-binap complex in crystalline porous frameworks (Zhou, H.-C., Long, J. R. & Yaghi, O. M. Introduction to metal-organic frameworks. Chem. Rev. 112, 673-674 (2012); Furukawa, H., Cordova, K. E., O'Keeffe, M. & Yaghi, O. M. The chemistry and applications of metal-organic frameworks. Science 341, 1230444 (2013)), the highly stable Universitetet i Oslo-69 (UiO-69) MOF (Cavka, J. H. et al. A new zirconium inorganic building brick forming metal organic frameworks with exceptional stability. J. Am. Chem. Soc. 130, 13850-13851 (2008); Newsome, W. J. et al. Solid state multicolor emission in substitutional solid solutions of metal-organic frameworks. J. Am. Chem. Soc. 141, 11298-11303 (2019)), which consists of [Zr₆] inorganic nodes and quaterphenyl dicarboxylate organic linkers, was chosen as a photocatalyst support. Considering that binap-ligated copper(I) complexes are intolerant of the solvothermal conditions for the MOF synthesis, a post-synthetic metalation method (Kalaj, M. & Cohen, S. M. Postsynthetic modification: an enabling technology for the advancement of metal-organic frameworks. ACS Cent. Sci. 6, 1046-1057 (2020); Li, J. et al. Self-adaptive dual-metal-site pairs in metal-organic frameworks for selective CO₂ photoreduction to CH₄. Nat. Catal. 4, 719-729 (2021); Ma, X. et al. Modulating coordination environment of single-atom catalysts and their proximity to photosensitive units for boosting MOF photocatalysis. J. Am. Chem. Soc. 143, 12220-12229 (2021)) was utilized rather than pre-synthetic modification of MOF linkers (Wang, C., Xie, Z., deKrafft, K. E. & Lin, W. Doping metal-organic frameworks for water oxidation, carbon dioxide reduction, and organic photocatalysis. J. Am. Chem. Soc. 133, 13445-13454 (2011); Wang, C., deKrafft, K. E. & Lin, W. Pt nanoparticles@photoactive metal-organic frameworks: efficient hydrogen evolution via synergistic photoexcitation and electron injection. J. Am. Chem. Soc. 134, 7211-7214 (2012)) to introduce the targeted copper species onto the UiO-69 MOF (FIG. 2a). To prevent a possible pore blockage during the metalation step, a small portion of the functionalized organic linker bearing a 1,10-phenanthroline (phen) moiety (i.e., 4,4′-(1,10-phenanthroline-3,8-diyl)dibenzoic acid) was mixed with a highly soluble tetramethyl-substituted quaterphenyl dicarboxylic acid in the synthesis of a phen-functionalized UiO-69 MOF (UiO-69-phen). The crystallinity and stability of UiO-69-phen are significantly superior to those of the one purely synthesized from the phen-containing linker. Its large Brunauer-Emmett-Teller (BET) surface area of 1,990 m² g⁻¹, comparable to that of the non-functionalized UiO-69 MOF³², and its pore width distribution centered at 1.6 nm pave the way for the subsequent metalation with cationic bisphosphine-chelated copper(I) complexes.

Immersing UiO-69-phen in a dichloromethane solution of [Cu(xantphos)(MeCN)₂](PF₆) or [Cu(binap)(MeCN)₂](PF₆) at room temperature produced MOF-supported heteroleptic copper photocatalyst UiO-69-phen(xantphos)Cu or UiO-69-phen(binap)Cu. When the same metalation conditions were applied to non-functionalized UiO-69 MOF without a phen-containing linker, no heterogeneous copper catalyst was produced. It is worth noting that the phen-functionalized UiO-67 MOF with a much smaller pore width distribution (Zhang, X. et al. Catalytic chemoselective functionalization of methane in a metal-organic framework. Nat. Catal. 1, 356-362 (2018)) was unable to accommodate the bulky cationic binap-ligated copper(I) complex even at elevated temperatures. Additionally, attempts to directly encapsulate the desired copper(I) species in conventional porous hosts, such as UiO-67 and UiO-68 MOFs, did not produce the corresponding heterogeneous binap-containing copper photosensitizers, presumably due to the low thermal stability of binap-ligated copper(I) complexes upon heating and their poor compatibility with strong Lewis acids. The heterogenized copper(I) species (UiO-69-phenCu) without chelation of bisphosphine ligands was prepared by treating UiO-69-phen with [Cu(MeCN)₄](PF₆) at 60° C. According to the inductively coupled plasma atomic emission spectroscopy (ICP-AES) and nuclear magnetic resonance (NMR) analysis, approximately one copper atom per [Zr₆] node was successfully loaded onto the MOF support in all three cases. The consistency of copper contents determined by ICP-AES and NMR analysis, along with the absence of other phosphine-containing species in the NMR digestion samples, ruled out the possibility of bisphosphine-chelated copper(I) complexes occluding the MOF pores during post-synthetic metalation. The N₂ adsorption isotherms for these copper-loaded MOFs suggested that the microporosity was preserved despite the decrease in the BET surface area. A typical octahedral morphology of UiO-69 MOFs was maintained upon post-synthetic metalation (SEM image not shown). The powder X-ray diffraction (PXRD) patterns revealed that the MOF crystallinity remained unchanged after the metalation processes (FIG. 2b). Energy-dispersive X-ray spectroscopy (EDS) mapping indicated the uniform incorporation of copper cations throughout the MOF crystallites. The Cu2p_{1/2} and Cu2p_{3/2} peaks in the X-ray photoelectron spectroscopy (XPS) spectra of the MOF-supported photocatalysts can be ascribed to copper(I) species (FIG. 2c). The minor shifts toward the lower binding energy region originate from the coordination of phenanthroline and bisphosphine ligands to the copper center.

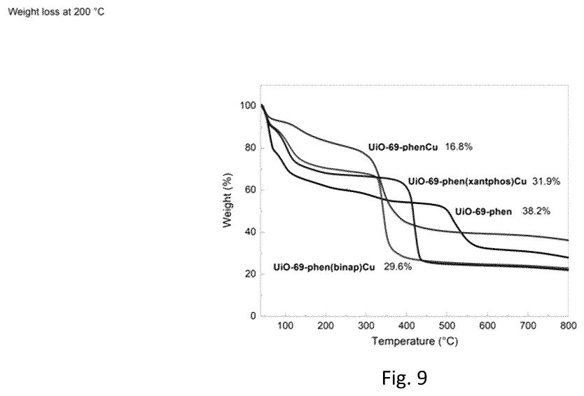

The copper coordination environments in the pores of UiO-69-phen(binap)Cu and UiO-69-phen(xantphos)Cu were further investigated using X-ray absorption spectroscopy at the Cu K-edge (FIG. 2d). To better understand the binding mode of the copper(I) species in the UiO-69 MOFs, the extended X-ray absorption fine structure (EXAFS) data of UiO-69-phen(binap)Cu was fitted to the crystal structure of Cu-1. The best fitting of the EXAFS regions suggests there are two Cu—N bonds at 1.86±0.01 Å and two Cu—P bonds at 2.15±0.01 Å, respectively (Table 2 below). Each copper(I) center of UiO-69-phen(binap)Cu adopts a distorted tetrahedral geometry. The thermal gravimetric analysis (TGA) (see FIG. 9) and ICP-AES results were used to determine the copper loading in each heterogeneous photosensitizer for exploring visible-light-mediated intermolecular crossed [2+2]cycloadditions.

TABLE 2
Summary of X-ray absorption fine structure fitting
results at the Cu K-edge for UiO-69-phen(binap)Cu

| | | | K: 3.0-10.0 Å⁻¹ |
|---|---|---|---|
| UiO-69-phen(binap)Cu | | Fitting Range | R: 1.0-2.0 Å |
| Reduced chi-square | 7388.9 | R-factor | 0.010 |
| ΔE₀ | −6.89 | S₀² | 1.0 |
| σ²Cu—N | 0.0001 | R(Cu—N) | 1.86 ± 0.01 |
| σ²Cu—P | 0.0066 | R(Cu—P) | 2.15 ± 0.01 |
| Independent points | 4.3 | Number of | 2 |
| | | variables | |
| | | CN(Cu—N) | 2 |
| | | CN(Cu—P) | 2 |

CN: coordination numbers; R: bond distance; σ²: Debye-Waller factors; ΔE₀: the inner potential correction; R factor: goodness of fit; S₀² was set as 1 for Cu data, which was obtained from the experimental EXAFS fit of Cu foil reference by fixing CN as the known crystallographic value and was fixed to all the samples.

### Photocatalytic Performance Characterization:

It was hypothesized that 1-vinylnaphthalene, having a relatively low triplet energy, would engage in energy transfer with photoexcited copper catalysts and then undergo an unprecedented [2+2]cycloaddition with methyl acrylate (Murray, P. R. D. et al. Intermolecular crossed [2+2]cycloaddition promoted by visible-light triplet photosensitization: expedient access to polysubstituted 2-oxaspiro[3.3]heptanes. J. Am. Chem. Soc. 143, 4055-4063 (2021); Coote, S. C. & Bach, T. Enantioselective intermolecular [2+2] photocycloadditions of isoquinolone mediated by a chiral hydrogen-bonding template. J. Am. Chem. Soc. 135, 14948-14951 (2013)) to afford trans-methyl 2-(naphthalen-1-yl)cyclobutane-1-carboxylate (1), as shown in Scheme 1 below.

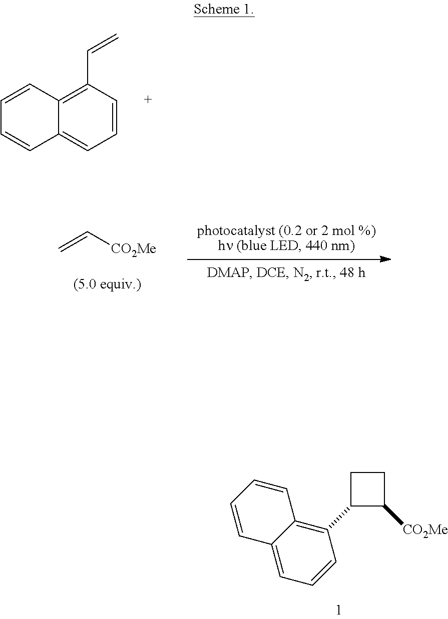

After a systematic study of various reaction parameters, a procedure was established that achieved the cycloaddition product using 0.2 mol % of UiO-69-phen(binap)Cu, with a turnover number (TON) of 365 and a diastereoselectivity of >10:1 (see Table 1, entry 1 below). Control experiments demonstrated that the reaction did not take place in the absence of light or photocatalysts. Other MOF-supported copper(I) complexes lacking binap coordination failed to provide any cycloaddition products (Table 1, entries 2 and 3), which indicates the formation of binap-based copper(I) photosensitizers is important for promoting the crossed [2+2]cycloaddition. The low reactivity of the homogeneous counterparts (Cu-1 and Cu-2) highlighted the significance of immobilizing the heteroleptic copper(I) complex on the MOF support in order to generate an effective copper photocatalyst (Table 1, entries 4-7). While a 10-fold increase in Cu-1 or Cu-2 loading gave a 7% yield of 1, the corresponding homogeneous xantphos-based copper(I) photosensitizers were ineffective (Table 1, entries 8 and 9). Changing the substituents of 1,10-phenanthroline in xantphos-ligated heteroleptic copper(I) complexes (Mejfa, E. et al. A noble-metal-free system for photocatalytic hydrogen production from water. Chem. Eur. J. 19, 15972-15978 (2013)) did not improve their photocatalytic activity in the [2+2]cycloaddition with methyl acrylate (Table 1, entries 10 and 11). The well-established [Ru(bpy)₃](PF₆)₂ photocatalyst, typically employed in the electron-transfer processes (Ischay, M. A., Lu, Z. & Yoon, T. P. [2+2]cycloadditions by oxidative visible light photocatalysis. J. Am. Chem. Soc. 132, 8572-8574 (2010)), was unable to yield the desired product (Table 1, entry 12). The homogeneous iridium triplet photosensitizers with higher transition energies (Table 1, entries 13 and 14) (Lei, T. et al. General and efficient intermolecular [2+2] photodimerization of chalcones and cinnamic acid derivatives in solution through visible-light catalysis. Angew. Chem. Int. Ed. 56, 15407-15410 (2017); Zhu, M., Zheng, C., Zhang, X. & You, S.-L. Synthesis of cyclobutane-fused angular tetracyclic spiroindolines via visible-light-promoted intramolecular dearomatization of indole derivatives. J. Am. Chem. Soc. 141, 2636-2644 (2019); Girvin, Z. C. et al. Asymmetric photochemical [2+2]-cycloaddition of acyclic vinylpyridines through ternary complex formation and an uncontrolled sensitization mechanism. J. Am. Chem. Soc. 144, 20109-20117 (2022), as well as their heterogeneous equivalents (Table 1, entries 15-18) (Wang, C., Xie, Z., deKrafft, K. E. & Lin, W. Doping metal-organic frameworks for water oxidation, carbon dioxide reduction, and organic photocatalysis. J. Am. Chem. Soc. 133, 13445-13454 (2011); Wang, C., deKrafft, K. E. & Lin, W. Pt nanoparticles@photoactive metal-organic frameworks: efficient hydrogen evolution via synergistic photoexcitation and electron injection. J. Am. Chem. Soc. 134, 7211-7214 (2012); Yu, X. & Cohen, S. M. Photocatalytic metal-organic frameworks for selective 2,2,2-trifluoroethylation of styrenes. J. Am. Chem. Soc. 138, 12320-12323 (2016); Fan, Y., Zheng, H., Labalme, S. & Lin, W. Molecular engineering of metal-organic layers for sustainable tandem and synergistic photocatalysis. J. Am. Chem. Soc. 145, 4158-4165 (2023)), are also incompatible with this reaction system, resulting in substantial substrate decomposition. Since the styrene substrate lacks a carboxylate substituent, it was not unexpected that CdSe quantum dots proved ineffective in the crossed [2+2]cycloaddition (Table 1, entry 19) (Jiang, Y., Wang, C., Rogers, C. R., Kodaimati, M. S. & Weiss, E. A. Regio- and diastereoselective intermolecular [2+2]cycloadditions photocatalysed by quantum dots. Nat. Chem. 11, 1034-1040 (2019)). Furthermore, a number of heterogeneous framework-based photocatalysts and homogeneous organic photocatalysts were investigated that could promote [2+2] dimerization of styrenes mediated by electron transfer; and none of them gave any desired products. The catalyst effect demonstrated that the binap-based copper triplet photosensitizer catalysts are capable of promoting intermolecular crossed [2+2]cycloadditions of styrenes with methyl acrylate, and successful integration of these photosensitizers on suitable MOF supports was shown to significantly boost their photocatalytic activity.

TABLE 1
Effect of different catalytic systems on the crossed
[2 + 2] cycloaddition

| Entry | Catalyst (mol %) | Yield^{a} (%) | TON^{b} |
|---|---|---|---|
| 1 | UiO-69-phen(binap)Cu (0.2) | 73 (>10:1 d.r.)^{c} | 365 |
| 2 | UiO-69-phenCu (0.2) | <5 | — |
| 3 | UiO-69-phen(xantphos)Cu (0.2) | <5 | — |
| 4 | Cu-1 (0.2) | <5 | — |
| 5 | Cu-2 (0.2) | <5 | — |
| 6 | Cu-1 (2) | 7 | 3.5 |
| 7 | Cu-2 (2) | 7 | 3.5 |
| 8 | Cu-3 (2) | <5 | — |
| 9 | Cu-4 (2) | <5 | — |
| 10 | [Cu(dmp)(xantphos)](PF₆) (2) | <5 | — |
| 11 | [Cu(bcp)(xantphos)](PF₆) (2) | <5 | — |
| 12 | [Ru(bpy)₃](PF₆)₂ (2) | <5 | — |
| 13 | Ir(ppy)₃ (2) | <5^{d} | — |
| 14 | [Ir(ppy^{F})₂(dtbbpy)](PF₆) (2) | <5^{d} | — |
| 15 | UiO-69-Ir(ppy)₂ (2) | <5^{d} | — |
| 16 | UiO-67-Ir(ppy)₂ (2) | <5^{d} | — |
| 17 | UiO-69-Ir(ppy^{F})₂ (2) | <5^{d} | — |
| 18 | MOL-Hf-Ir(ppy^{F})₂ (2) | <5^{d} | — |
| 19 | CdSe QDs (2) | <5 | — |

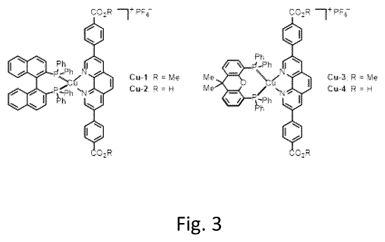

Conditions: 1-vinylnaphthalene (0.2 mmol, 1.0 equiv.), methyl acrylate (5.0 equiv.), photocatalyst (0.2 or 2 mol %), and DMAP (1.2 equiv.) in anhydrous DCE (1 ml) under nitrogen atmosphere at room temperature with blue-LED light irradiation (440 nm) for 48 h. DMAP, 4-dimethylaminopyridine; DCE, 1,2-dichloroethane; dmp, 2,9-dimethyl-1,10-phenanthroline; bcp, bathocuproine; bpy, 2,2′-bipyridine; ppy, 2-(2-pyridyl)phenyl; dtbbpy, 4,4′-di-tert-butyl-2,2′-bipyridine; ppy^{F}, 3,5-difluoro-2-(5-trifluoromethyl-2-pyridyl)phenyl; MOL-Hf—Ir(ppy^{F})₂, a metal-organic layer containing [Hf₁₂] inorganic nodes and iridium-functionalized dicarboxylate organic linkers⁴⁸. ^{a}Yield was determined by ¹H NMR of the crude product using phenyltrimethylsilane as an internal standard. ^{b}TONs shown in Table 1 were calculated based on the crude NMR yield of 1. For other products, TONs were calculated based on isolated yields. ° Diastereomeric ratio (d.r.) was determined by ¹H NMR analysis of the crude reaction mixture. ^{d}1-Vinylnaphthalene was fully decomposed. The chemical structures of Cu-1, Cu-2, Cu-3, and Cu-4 are shown in FIG. 3.

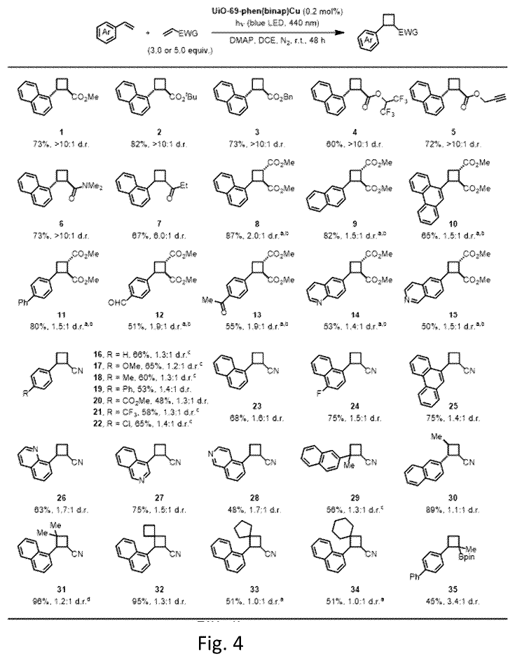

Copper photocatalyst UiO-69-phen(binap)Cu was examined on a variety of electron-deficient alkenes to evaluate the scope of the heterogeneous photocatalytic [2+2]cycloaddition reactions (FIG. 4). A number of α,β-unsaturated carbonyl compounds, such as acrylates, acrylamides, vinyl alkyl ketones, and fumarates, are highly reactive, furnishing the crossed cycloaddition products in yields of up to 87% (FIG. 4, products 1-15). In the crossed cycloadditions of various styrenes, the required amount of dimethyl fumarate can be reduced to 1.2 equivalents (FIG. 4, products 8-15), likely owing to the more electron-deficient nature of its double bond. To further investigate the styrene scope, acrylonitrile was chosen as the reaction partner. It is important to note that simple styrenes with different para-substituents delivered the cyclobutane products in good yields (FIG. 4, products 16-22), despite the use of 1.5 mol % of UiO-69-phen(binap)Cu for the substrates with higher triplet energies (Ni, T., Caldwell, R. A. & Melton, L. A. The relaxed and spectroscopic energies of olefin triplets. J. Am. Chem. Soc. 111, 457-464 (1989)). Other olefins that bear fused aromatic rings, including quinolines and isoquinolines, participated in the crossed [2+2]cycloadditions with high reactivity (FIG. 4, products 23-28). Notably, in addition to the terminal styrenes, α- and β-methyl substituted 2-vinylnaphthalenes (FIG. 4, products 29 and 30), as well as β,β-disubstituted 1-vinylnaphthalenes (FIG. 4, products 31-34), are suitable substrates for the heterogeneous copper photocatalysis. In the synthesis of spiro-cycloadducts, the substrates are not limited to arylidene 4-membered rings: the 5- and 6-membered ring analogues also successfully yielded the desired products with 0.75 mol % of UiO-69-phen(binap)Cu. Moreover, isopropenylboronic acid pinacol ester, a 1,1-disubstituted electron-deficient alkene, gave a trisubstituted cyclobutane with a boryl group that is easily convertible to other useful functionalities (He, J., Shao, Q., Wu, Q. & Yu, J.-Q. Pd(II)-catalyzed enantioselective C(sp³)-H borylation. J. Am. Chem. Soc. 139, 3344-3347 (2017)) (FIG. 4, product 35). To demonstrate the practicality of the method, cycloadduct 1 of FIG. 4 was synthesized on a gram scale (70% yield) using 0.15 mol % of the heterogeneous copper photocatalyst.

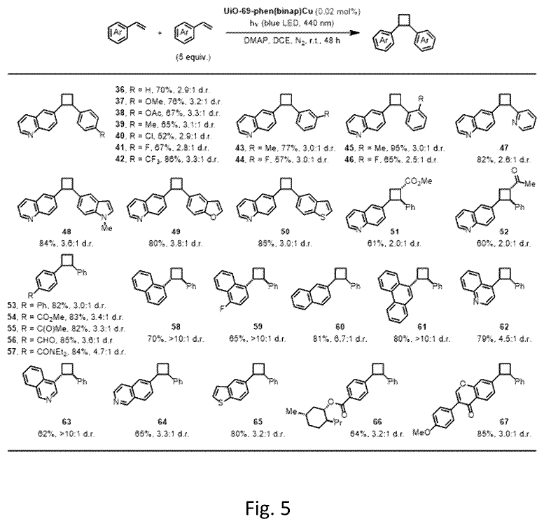

With respect to the [2+2]cycloadditions between different styrenes, the heterogeneous copper photocatalysis proved to be more efficient (FIG. 5). Only 0.02 mol % of UiO-69-phen(binap)Cu was needed to obtain synthetically useful yields, resulting in high TONs of up to 4750. To exploit the scope of styrene coupling partners, as well as to build a library of potentially bioactive quinoline derivatives, 6-vinylquinoline was selected as the limiting reagent in the copper-catalyzed photocycloaddition. Functional groups with different electronic properties are well tolerated at the ortho-, meta-, orpara-positions of styrenes (FIG. 5, products 36-46). Diverse heterocycles (e.g., pyridine, indole, benzofuran, and benzothiophene) can be conveniently incorporated into the final cycloadducts (FIG. 5, products 47-50). The crossed cycloadditions with methyl cinnamate and benzylideneacetone afforded the corresponding trisubstituted cyclobutanes with high regioselectivity (FIG. 5, products 51 and 52).

During the photosensitization of other styrenes, the unsubstituted simple styrene was chosen as the coupling partner to determine the reaction scope. A wide range of substituted aryl groups and heterocyclic moieties were found to be compatible with this visible light photocatalysis (FIG. 5, products 53-65). It is noteworthy that there was a substantial increase in diastereoselectivity (>10:1 d.r.) when a bulky 1-naphthyl, 9-phenanthryl, or 4-isoquinolyl group was introduced to the substrate (FIG. 5, products 58, 59, 61, and 63). To further showcase the utility of this methodology, the substrates derived from bioactive molecules (i.e., (+)-menthol and formononetin) were utilized in the crossed [2+2]cycloadditions, giving the desired products in 64% and 85% yields, respectively (FIG. 5, products 66 and 67).

The exceptional photocatalytic activity of UiO-69-phen(binap)Cu prompted us to revisit the intermolecular crossed [2+2]cycloadditions of exocyclic arylidene 4-membered rings (other than cyclobutanes) with electron-deficient alkenes (FIG. 6), previously explored by Knowles and co-workers using a potent iridium(III) photosensitizer (Murray, P. R. D. et al. Intermolecular crossed [2+2]cycloaddition promoted by visible-light triplet photosensitization: expedient access to polysubstituted 2-oxaspiro[3.3]heptanes. J. Am. Chem. Soc. 143, 4055-4063 (2021)). Under the same conditions established for the simple styrenes at low catalyst loadings (FIG. 4), various azetidine, thietane, and oxetane substrates reacted smoothly with acrylonitrile to give the corresponding 2-azaspiro[3.3]heptane, 2-thiaspiro[3.3]heptane, 2-oxaspiro[3.3]heptane products in good to excellent yields (FIG. 6, products 68-76), including several challenging substrates in the homogeneous iridium photocatalysis (FIG. 6, products 69 and 71). Similar to the situation in the synthesis of 58, the reaction of 3-(1-naphthylmethylene)oxetane is highly diastereoselective. The scope with regard to the electron-deficient alkenes is also fairly broad (FIG. 6, products 77-85). In addition to the aforementioned examples, α-acetamidoacrylate and α-fluoroacrylate can serve as effective coupling partners in the crossed cycloaddition, providing a straightforward method for the preparation of 2-oxaspiro[3.3]heptanes containing α-amino acid moieties and fluorine atoms (FIG. 6, products 81 and 82).

The coupling partner scope in the heterogeneous copper photocatalytic system can be further expanded to include a wide array of styrenes that have yet to be investigated (FIG. 6). Electron-donating or -withdrawing groups at various positions of the aryl ring had no obvious impact on the reaction yield (FIG. 6, products 86-92). All the 2-oxaspiro[3.3]heptane products bearing heteroaryl groups, such as pyridyl (93), indolyl (94), benzofuranyl (95), and benzothienyl (96), can be synthesized efficiently; see FIG. 6. The cis-isomer of 93 was completely converted into the trans-isomer upon column purification with silica gel, possibly due to the ring-opening isomerization triggered by protonation of the pyridine moiety. This reaction protocol also works well with 1,1-diphenylethylene, affording a triaryl substituted 2-oxaspiro[3.3]heptane (97) in 83% yield, as shown in FIG. 6.

### Stability and Recyclability Tests:

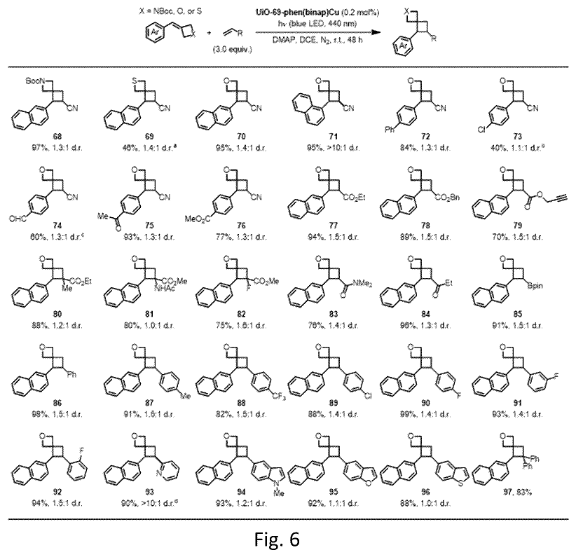

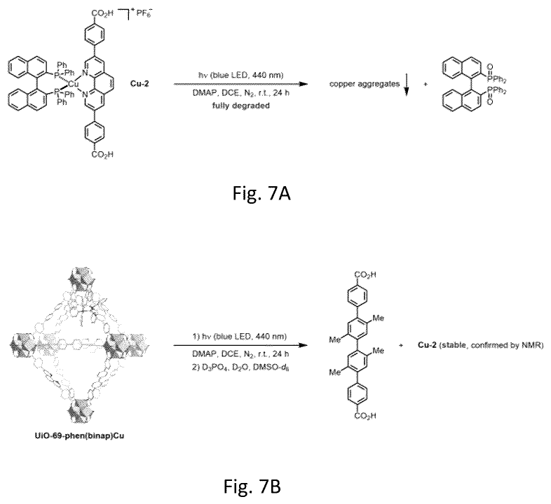

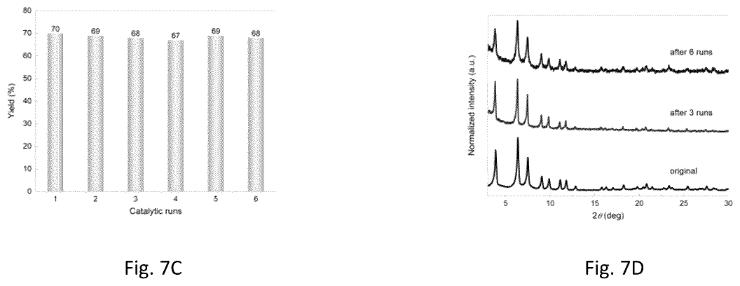

The significance of employing a heterogenization strategy in the design of binap-based copper photosensitizer catalysts was elucidated by control experiments comparing the photostability of Cu-2 and UiO-69-phen(binap)Cu under the standard reaction conditions without substrates (FIGS. 7a and 7b). Upon blue-LED light irradiation for 24 hours, the heteroleptic copper(I) complex (Cu-2), which serves as the copper-functionalized linker for the UiO-69 MOF, was completely degraded into insoluble aggregates along with binap dioxide. In contrast, UiO-69-phen(binap)Cu exhibited very high stability when exposed to such light: Cu-2 was detected by NMR after MOF digestion with deuterated phosphoric acid in a solvent mixture of deuterated water and dimethyl sulfoxide; the ratio between Cu-2 and the normal linker remained constant before and after light irradiation. Due to its heterogeneity and high photostability, UiO-69-phen(binap)Cu can be easily recovered by filtration and reused at least six times without any loss of its photocatalytic activity (FIG. 7c). The PXRD patterns (FIG. 7d), microscopic images, and N₂ adsorption isotherms of the recovered copper photocatalysts after six cycles of [2+2]cycloadditions indicated that UiO-69-phen(binap)Cu retained its crystalline structure after photocatalysis.

### Mechanistic Studies:

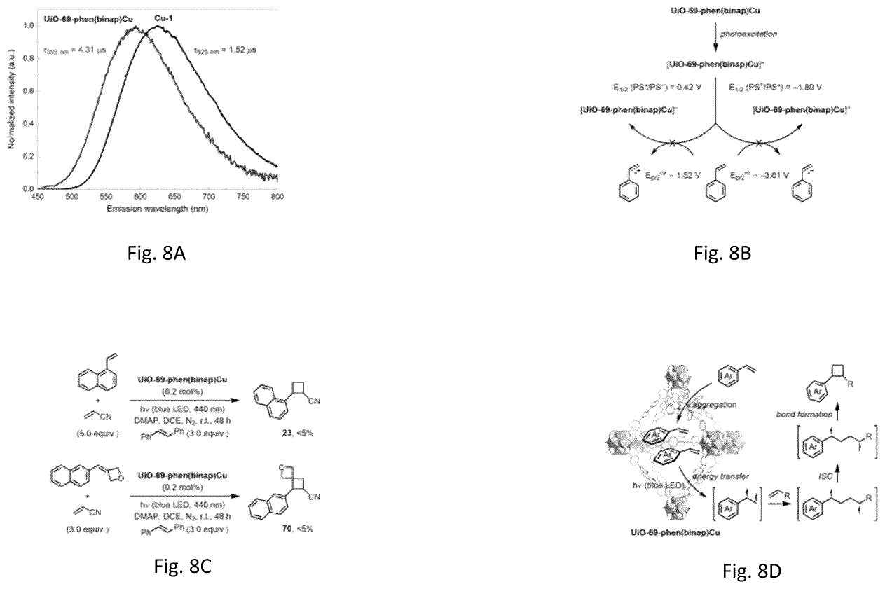

Upon post-synthetic metalation with the cationic binap-ligated copper(I), the maximum absorption peak was observed at 449 nm, resulting from metal-to-ligand charge transfer transitions, appeared alongside the original peak at 351 nm from the MOF support (UiO-69-phen) in the solid-state absorption spectrum of UiO-69-phen(binap)Cu. Compared to other heterogeneous copper photocatalysts, the absorption profile of UiO-69-phen(binap)Cu showed the highest intensity in the region of blue-LED excitation. Notably, the phosphorescence peak of the heterogenized copper(I) species is blue-shifted by 33 nm from that of its homogeneous counterpart, with an increased excited-state lifetime at room temperature from 1.52 to 4.31 μs (FIG. 8a). In the first triplet excited state (T₁) of heteroleptic copper complexes, the copper center tends to adopt square-planar coordination, as opposed to the tetrahedral coordination observed in the ground state (S₀). The geometrical restriction imposed by the octahedral UiO-69 MOF raises the energy of the T₁ state and impedes the T₁→S₀ transition. The increases in transition energies (from 45.7 to 48.3 kcal mol⁻¹) and excited-state lifetimes (nearly three folds) could greatly facilitate the energy-transfer processes in the intermolecular crossed [2+2]cycloadditions of styrenes. On the other hand, based on the estimated excited-state redox potentials of UiO-69-phen(binap)Cu, the single-electron transfer between the copper active site and the styrene substrate is strongly disfavored (Roth, H. G., Romero, N. A. & Nicewicz, D. A. Experimental and calculated electrochemical potentials of common organic molecules for applications to single-electron redox chemistry. Synlett 27, 714-723 (2016)) (FIG. 8b). Furthermore, using trans-stilbene as a triplet-energy-transfer inhibitor (Cheng, X., Li, T., Liu, Y. & Lu, Z. Stereo- and enantioselective benzylic C—H alkenylation via photoredox/nickel dual catalysis. ACS Catal. 11, 11059-11065 (2021)), it was found that the crossed cycloadditions with acrylonitrile were completely suppressed; no cycloadducts were detected (FIG. 8c).

It was shown that 1-vinylnaphthalene quenches the luminescence of UiO-69-phen(binap)Cu but not UiO-69-phen(xantphos)Cu, demonstrating the superior activity of UiO-69-phen(binap)Cu in facilitating triplet energy transfer from photoexcited copper(I) to styrenes. In addition, the rate order of simple styrene was determined to be 4.28±0.40, indicating that substrate aggregation occurred prior to photoexcitation. Taken together, the following catalytic cycle may be operative (FIG. 8d). The styrene substrate aggregates in the MOF pores to form an exciplex with a much lower transition energy (Liu, Z. et al. Aggregation-enabled intermolecular photo[2+2]cycloaddition of aryl terminal olefins by visible-light catalysis. CCS Chem. 2, 582-588 (2020)), which is then sensitized by the excited-state copper(I) photocatalyst via energy transfer. The resulting 1,2-triplet styrene undergoes an intermolecular radical addition with an electron-deficient alkene or a simple styrene to generate a 1,4-triplet diradical. The intersystem crossing (ISC) followed by intramolecular radical recombination yields the [2+2]cycloaddition product.

### Conclusions

This example detailed for the synthesis and use of well-defined crystalline MOFs to support heteroleptic copper(I) complexes in order to facilitate a series of intermolecular crossed [2+2]cycloadditions via energy transfer under visible light irradiation. The incorporation of binap-containing copper triplet photosensitizers onto the MOF organic linkers substantially improved the photostability and increased the excited-state lifetime which allowed cycloadditions of various styrenes with electron-deficient alkenes. This heterogeneous copper photocatalysis demonstrated broad substrate scope, excellent catalytic efficiency, and remarkable catalyst recyclability, as well a general approach for designing highly reactive copper photocatalysts for diverse energy-transfer-mediated organic transformations.

In particular, it was shown that associating a binap-ligated heteroleptic copper(I) complex to the linker of a microporous zirconium-based metal-organic framework (MOF) produced a highly stable and reusable heterogeneous photocatalyst with an extended excited-state lifetime. Under visible light irradiation, this copper triplet photosensitizer efficiently promoted multiple intermolecular crossed [2+2]cycloadditions, including a cycloaddition reaction of simple styrenes with electron-deficient alkenes.

### Additional Example 1 Details

### Synthesis of Organic Linkers:

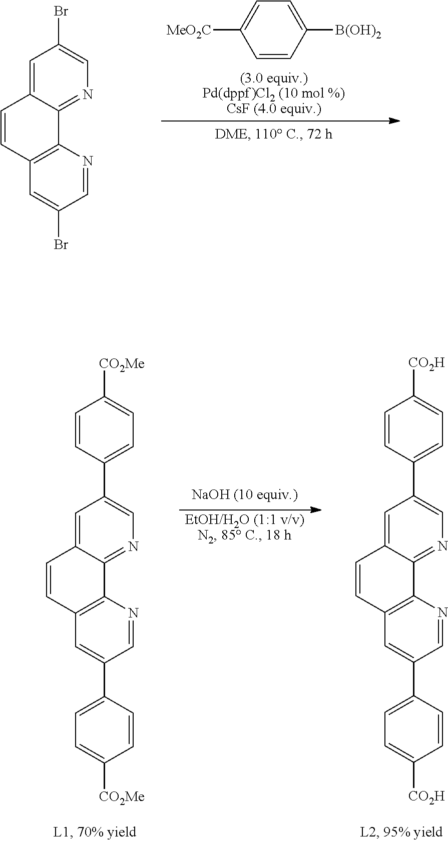

Dimethyl 4,4′-(1,10-phenanthroline-3,8-diyl)dibenzoate (Li). To a solution of 3,8-dibromo-1,10-phenanthroline (3.4 g, 10 mmol, 1.0 equiv.) in dimethoxyethane (150 mL), 4-(methoxycarbonyl)phenylboronic acid (5.4 g, 30 mmol, 3.0 equiv.), Pd(dppf)Cl₂ (0.732 g, 1.0 mmol, 10 mol %), and CsF (6.1 g, 40 mmol, 4.0 equiv.) were added. After stirring at 110° C. for 72 hours under N₂ atmosphere, the solid was collected by centrifugation and washed with water, chloroform and cold acetone, respectively. Then resulted crude solid was further purified by Soxhlet extraction with chloroform for 48 hours. The extraction was concentrated in vacuo to give 3.1 g (7.0 mmol, 70% yield) of the title compound as a white solid.

¹H NMR (CDCl₃, 400 MHz): δ 9.48 (s, 2H), 8.48 (s, 2H), 8.24 (d, J=8.0 Hz, 4H), 7.94 (s, 2H), 7.88 (d, J=8.0 Hz, 4H), 3.99 (s, 6H).

¹³C NMR (CDCl₃, 100 MHz): δ 166.7, 151.5, 149.3, 141.8, 134.9, 134.0, 130.5, 130.1, 128.6, 127.5, 127.4, 52.3.

HRMS (ESI) calculated for C₂₈H₂₀N₂O₄ [M+H]⁺ requires m/z 449.1496, found m/z 449.1421.

4,4′-(1,10-Phenanthroline-3,8-diyl)dibenzoic acid (L2). To a suspension of L1 (224 mg, 0.5 mmol, 1.0 equiv.) in ethanol (5.0 mL), NaOH (200 mg, 5 mmol, 10 equiv.) dissolved in water (5.0 mL) was added dropwise. The mixture was refluxed at 85° C. for 18 hours under N₂ atmosphere. After cooling to room temperature, the solution was acidified to pH ˜5 with 1 M HCl. The solid was collected by filtration, washed with water, ethanol and dried in vacuo to afford 200 mg (0.475 mmol, 95% yield) of the title compound as a pale yellow solid.

¹H NMR (CDCl₃, 400 MHz): δ 13.12 (brs, 2H), 9.51 (d, J=2.0 Hz, 2H), 8.94 (d, J=2.0 Hz, 2H), 8.19-8.08 (m, 10H).

¹³C NMR (CDCl₃, 100 MHz): δ 167.0, 134.2, 130.6, 130.2, 127.6, 127.5.

HRMS (ESI) calculated for C₂₆H₁₆N₂O₄ [M+H]⁺ requires m/z 421.1183, found m/z 421.1127.

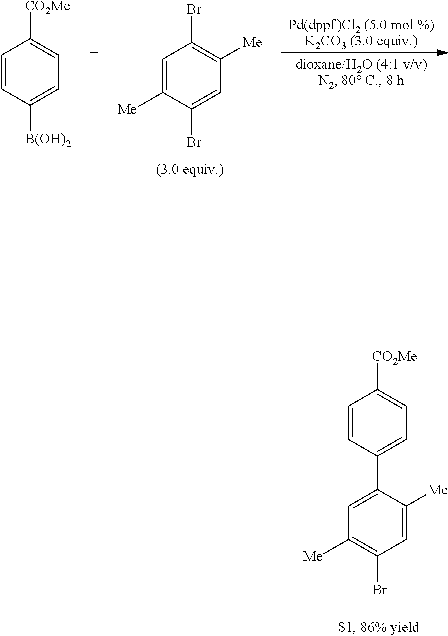 [SMILES: COC(=O)c1ccc(B(O)O)cc1 ; Cc1cc(Br)c(C)cc1Br ; "Pd(dppf)Cl2 (5.0 mol %)K2CO3 (3.0 equiv.)" ; "dioxane/H2O (4:1 v/v)N2, 80° C., 8 h" ; COC(=O)c1ccc(-c2cc(C)c(Br)cc2C)cc1]

4′-Bromo-2′,5′-dimethyl-[1,1′-biphenyl]-4-carboxylate (S1). To a suspension of 4-(methoxycarbonyl)phenylboronic acid (1.8 g, 10 mmol, 1.0 equiv.), 1,4-dioxane (48 mL), and water (12 mL), 1,4-dibromo-2,5-dimethyl-benzene (7.9 g, 30 mmol, 3.0 equiv.), Pd(dppf)Cl₂ (0.37 g, 0.5 mmol, 5.0 mol %), and K₂CO₃ (4.1 g, 30 mmol, 3.0 equiv.) were added. After stirring at 80° C. for 8 hours under N₂ atmosphere, the reaction mixture was diluted with water, and the aqueous layer was extracted with ethyl acetate (3×50 mL). The combined organic layers were washed with water and brine, dried over anhydrous sodium sulfate, and concentrated in vacuo. The crude mixture was purified by flash column chromatography using hexane/ethyl acetate=10:1 as the eluent to give 2.7 g (8.6 mmol, 86% yield) of the title compound as a white solid.

¹H NMR (CDCl₃, 400 MHz): δ 8.08 (d, J=8.4 Hz, 2H), 7.46 (s, 1H), 7.36 (d, J=8.4 Hz, 2H), 7.08 (s, 1H), 3.94 (s, 3H), 2.39 (s, 3H), 2.19 (s, 3H).

¹³C NMR (CDCl₃, 100 MHz): δ 166.9, 145.6, 140.0, 135.2, 134.4, 133.9, 131.7, 129.5, 129.1, 128.8, 124.1, 52.2, 22.3, 19.6.

HRMS (ESI) calculated for C₁₆H₁₅BrO₂[M+H]⁺ requires m/z 319.0328, found m/z 319.0306.

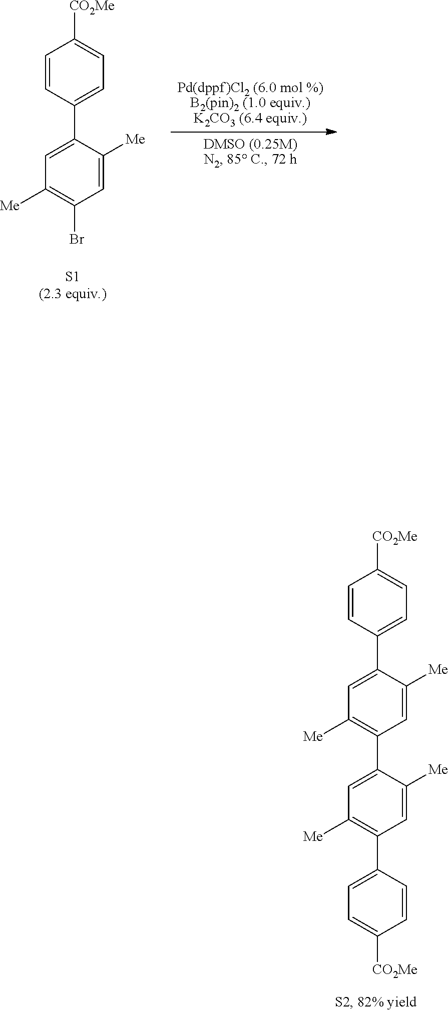 [SMILES: COC(=O)c1ccc(-c2cc(C)c(Br)cc2C)cc1 ; "DMSO (0.25M)N2, 85° C., 72 h" ; "Pd(dppf)Cl2 (6.0 mol %)B2(pin)2 (1.0 equiv.)K2CO3 (6.4 equiv.)" ; COC(=O)c1ccc(-c2cc(C)c(-c3cc(C)c(-c4ccc(C(=O)OC)cc4)cc3C)cc2C)cc1]

Dimethyl 2′,2″,5′,5″-tetramethyl-[1,1′:4′,1″:4″,1″′-quaterphenyl]-4,4″′-dicarboxylate (S2). To a solution of S1 (7.3 g, 23 mmol, 2.3 equiv.) in dimethyl sulfoxide (40 mL), bis(pinacolato)diboron (2.5 g, 10 mmol, 1.0 equiv.), Pd(dppf)Cl₂ (0.439 g, 0.6 mmol, 6.0 mol %), and K₂CO₃ (8.8 g, 64 mmol, 6.4 equiv.) were added. After stirring at 85° C. for 72 hours under N₂ atmosphere, the reaction mixture was diluted with water, and the aqueous layer was extracted with ethyl acetate (3×60 mL). The combined organic layers were washed with water and brine, dried over anhydrous sodium sulfate, and concentrated in vacuo. The crude mixture was purified by flash column chromatography using hexane/ethyl acetate=9:1 as the eluent to give 3.3 g (8.2 mmol, 82% yield) of the title compound as a white solid.

¹H NMR (CDCl₃, 400 MHz): δ 8.11 (d, J=8.2 Hz, 4H), 7.48 (d, J=8.2 Hz, 4H), 7.15 (s, 2H), 7.07 (s, 2H), 3.96 (s, 6H), 2.27 (s, 6H), 2.13 (s, 6H).

¹³C NMR (CDCl₃, 100 MHz): δ 167.1, 146.7, 140.8, 139.6, 133.4, 132.1, 131.5, 131.0, 129.4, 129.3, 128.5, 52.1, 19.9, 19.4.

HRMS (ESI) calculated for C₃₂H₃₀O₄[M+H]⁺ requires m/z 479.2217, found m/z 479.2195.

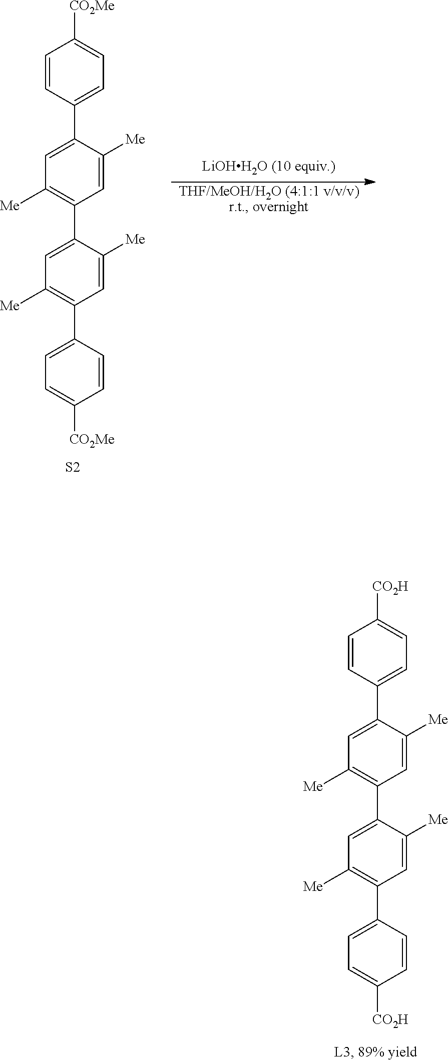

2′,2″,5′,5″-Tetramethyl-[1,1′:4′,1″:4″,1″′-quaterphenyl]-4,4′″-dicarboxylic acid (L3). To a solution of S2 (2.8 g, 6.0 mmol, 10 equiv.) in a mixed solvent of tetrahydrofuran (96 mL), methanol (24 mL), and water (24 mL), LiOH·H₂O (2.5 g, 60 mmol, 10 equiv.) was added. After stirring at room temperature overnight, the solution was acidified to pH ˜5 with 1 M HCl. Then solvent was removed. The resulted solid was collected via filtration and washed with water and dried in vacuo to afford 2.4 g (5.3 mmol, 89% yield) of the title compound as a white solid.

¹H NMR (DMSO-d₆, 400 MHz): δ 12.99 (brs, 2H), 8.03 (d, J=8.2 Hz, 4H), 7.55 (d, J=8.2 Hz, 4H), 7.20 (s, 2H), 7.08 (s, 2H), 2.25 (s, 6H), 2.08 (s, 6H).

¹³C NMR (DMSO-d₆, 100 MHz): δ 167.2, 145.6, 140.3, 139.2, 132.8, 131.8, 131.3, 130.8, 129.3, 129.2, 19.6, 19.0.

HRMS (ESI) calculated for C₃₀H₂₆O₄[M+H]⁺ requires m/z 451.1904, found m/z 451.1837.

### Synthesis of Copper Complexes:

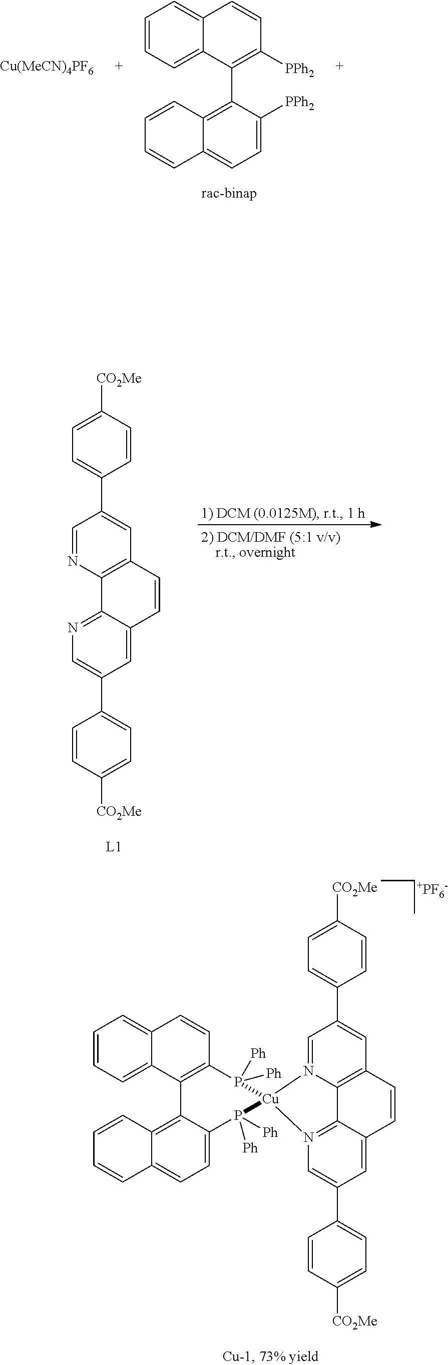

Cu-1. To a 50-mL flame-dried Schlenk flask cooled under N₂, Cu(MeCN)₄PF₆ (93 mg, 0.25 mmol), [1,1′-binaphthalene]-2,2′-diyl)bis(diphenylphosphane) (rac-binap) (156 mg, 0.25 mmol, 1.0 equiv.), and dichloromethane (20 mL) were added sequentially. After stirring at room temperature for 1 hour, Li (105 mg, 0.25 mmol, 1.0 equiv.) in 15 mL of mixed solvents of dichloromethane and dimethylformamide (5:1 v/v) was added in dropwise. Then, the whole solution was stirred at room temperature overnight. After the reaction was completed, dichloromethane was removed in vacuo. The residue was diffused into diethyl ether (80 mL) dropwise and the resulted yellow solid (229 mg, 0.183 mmol, 73% yield) was collected via filtration, washed by diethyl ether for several times and dried in vacuo.

¹H NMR (CDCl₃, 400 MHz): δ 8.90 (s, 2H), 8.53 (s, 2H), 8.36 (s, 2H), 8.23 (d, J=8.2 Hz, 4H), 7.63 (d, J=8.2 Hz, 2H), 7.59-7.51 (m, 6H), 7.46 (t, J=7.4 Hz, 2H), 7.36-7.11 (m, 14H), 7.08-7.00 (m, 4H), 6.92 (t, J=7.4 Hz, 2H), 6.78 (d, J=8.4 Hz, 2H), 6.70 (t, J=7.4 Hz, 4H), 4.02 (s, 6H).

³¹P NMR (CDCl₃, 162 MHz): δ 1.7, −135.4-−153.0 (m, 1P).

¹³C NMR (CDCl₃, 100 MHz): δ 166.6, 148.8, 142.2 (virtual triplet, J_{PC}=1.6 Hz), 139.9, 139.8 (virtual triplet, J_{PC}=10.0 Hz), 136.7, 135.9, 134.1 (virtual triplet, J_{PC}=4.2 Hz), 133.9 (virtual triplet, J_{PC}=9.2 Hz), 133.20, 133.15 (virtual triplet, J_{PC}=8.2 Hz), 131.7 (virtual triplet, J_{PC}=17.7 Hz), 130.7, 130.3 (virtual triplet, J_{PC}=13.8 Hz), 130.0, 129.3 (virtual triplet, J_{PC}=5.0 Hz), 129.1 (virtual triplet, J_{PC}=2.9 Hz), 128.7, 128.3 (virtual triplet, J_{PC}=16.5 Hz), 128.2, 127.7 (virtual triplet, J_{PC}=5.3 Hz), 127.5, 127.4 (virtual triplet, J_{PC}=3.5 Hz), 127.23 (virtual triplet, J_{PC}=29.9 Hz), 127.21 52.5.

FT-IR (film): 2923, 1717, 1431, 1280, 1106, 838, 748, 558 cm⁻¹.

HRMS (ESI) calculated for [C₇₂H₅₂CuN₂O₄P₂]⁺ requires m/z 1133.2693, found m/z 1133.2693.

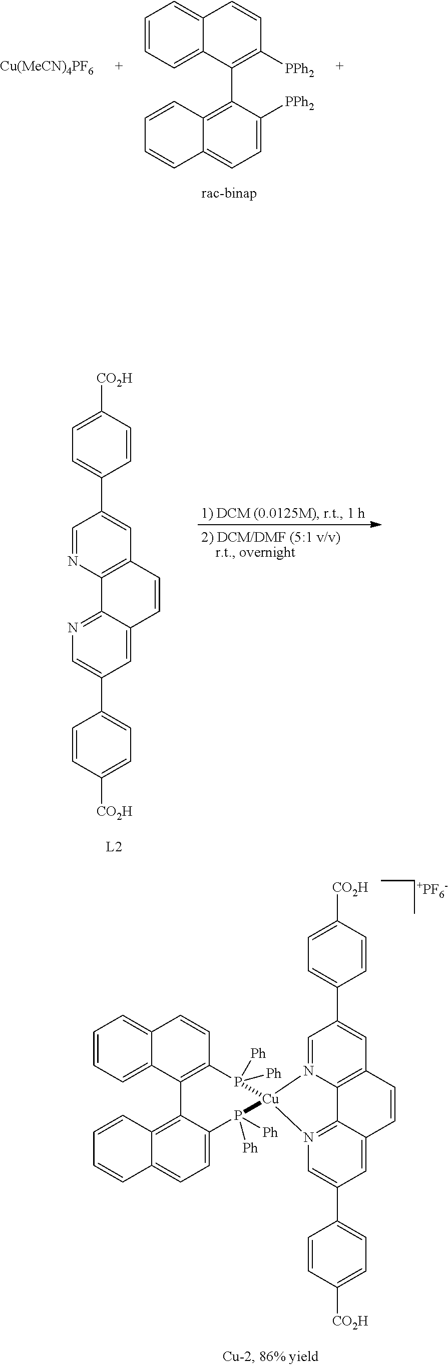

Cu-2. To a 50-mL flame-dried Schlenk flask cooled under N₂, Cu(MeCN)₄PF₆ (93 mg, 0.25 mmol), dichloromethane (20 mL), and rac-binap (156 mg, 0.25 mmol, 1.0 equiv.) were added sequentially. After stirring at room temperature for 1 hour, L2 (112 mg, 0.25 mmol, 1.0 equiv.) in dichloromethane and dimethylformamide (5:1 v/v) was added in dropwise. Then, the whole solution was stirred at room temperature overnight. After the reaction was completed, dichloromethane was removed in vacuo. The residue was diffused into diethyl ether (50 mL) dropwise and the resulted yellow solid (276 mg, 0.215 mmol, 86% yield) was collected via filtration, washed by diethyl ether for several times and dried in vacuo.

¹H NMR (DMSO-d₆, 400 MHz): δ 13.2 (brs, 2H), 9.27 (s, 2H), 8.78 (s, 2H), 8.40 (s, 2H), 8.16 (d, J=7.6 Hz, 4H), 7.85-7.66 (m, 8H), 7.49-7.06 (m, 20H), 6.91 (s, 2H), 6.76-6.65 (m, 6H).

³¹P NMR (DMSO-d₆, 162 MHz): δ 2.3, −135.4-−153.0 (m, 1P).

FT-IR (film): 2973, 1689, 1276, 1069, 838, 743, 693 cm⁻¹.

HRMS (ESI) calculated for [C₇₀H₄₈CuN₂O₄P₂]⁺ requires m/z 1105.2380, found m/z 1105.2373.

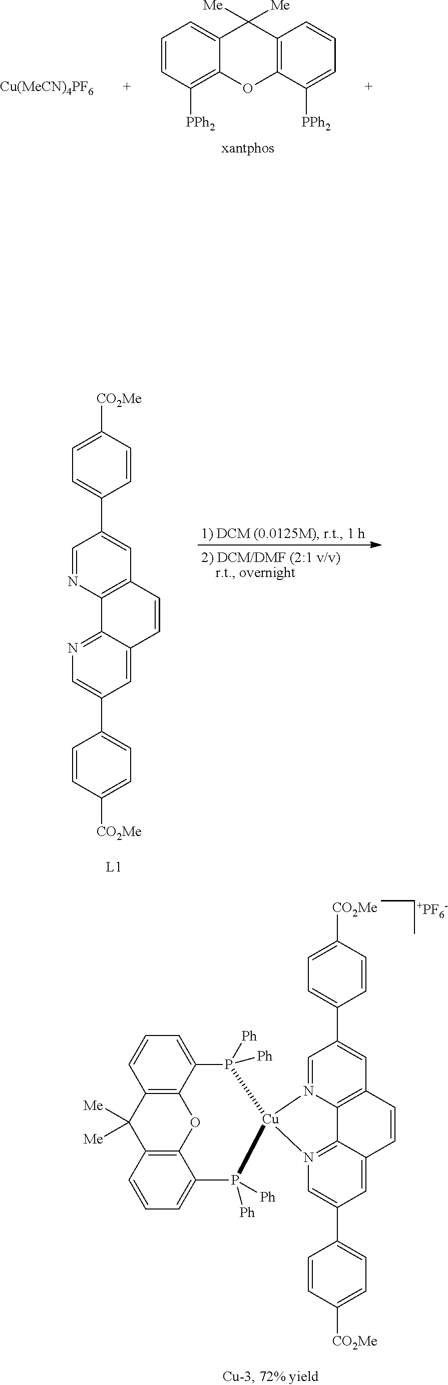

Cu-3. To a 50-mL flame-dried Schlenk flask cooled under N₂, Cu(MeCN)₄PF₆ (93 mg, 0.25 mmol), (9,9-dimethyl-9H-xanthene-4,5-diyl)bis(diphenylphosphane) (xantphos) (146 mg, 0.25 mmol, 1.0 equiv.), and dichloromethane (20 mL) were added sequentially. After stirring at room temperature for 1 hour, L1 (105 mg, 0.25 mmol, 1.0 equiv.) in 15 mL of mixed solvents of dichloromethane and dimethylformamide (2:1 v/v) was added in dropwise. Then, the whole solution was stirred at room temperature overnight. After the reaction was completed, dichloromethane was removed in vacuo. The residue was diffused into diethyl ether (80 mL) dropwise and the resulted orange solid (216 mg, 0.18 mmol, 72% yield) was collected via filtration, washed by diethyl ether for several times and dried in vacuo.

¹H NMR (DMSO-d₆, 400 MHz): δ 9.13 (s, 2H), 8.69 (s, 2H), 8.30 (s, 2H), 8.13 (d, J=8.4 Hz, 4H), 7.91 (d, J=7.2 Hz, 2H), 7.61 (d, J=8.4 Hz, 4H), 7.28 (t, J=7.6 Hz, 6H), 7.13 (t, J=7.6 Hz, 8H), 7.04-6.91 (m, 8H), 6.64-6.54 (m, 2H), 3.93 (s, 6H), 1.70 (s, 6H).

³¹P NMR (DMSO-d₆, 162 MHz): δ −11.4, −124.7-159.9 (m, 1P).

¹³C NMR (DMSO-d₆, 100 MHz): δ 165.8, 154.5 (virtual triplet, J_{PC}=6.4 Hz), 147.4, 141.7 (virtual triplet, J_{PC}=1.8 Hz), 139.7, 135.9, 135.5, 134.0, 132.5 (virtual triplet, J_{PC}=8.0 Hz), 131.0, 130.7 (virtual triplet, J_{PC}=17.0 Hz), 130.1 (two carbon signals overlapped), 130.0, 129.4, 128.8 (virtual triplet, J_{PC}=4.7 Hz), 128.2, 127.9, 127.7, 125.6, 119.1 (virtual triplet, J_{PC}=13.6 Hz), 52.5, 36.0, 27.7.

FT-IR (film): 2933, 1708, 1231, 845, 746, 670 cm⁻¹.

HRMS (ESI) calculated for [C₆₇H₅₂CuN₂O₅P₂]⁺ requires m/z 1089.2642, found m/z 1089.2258.

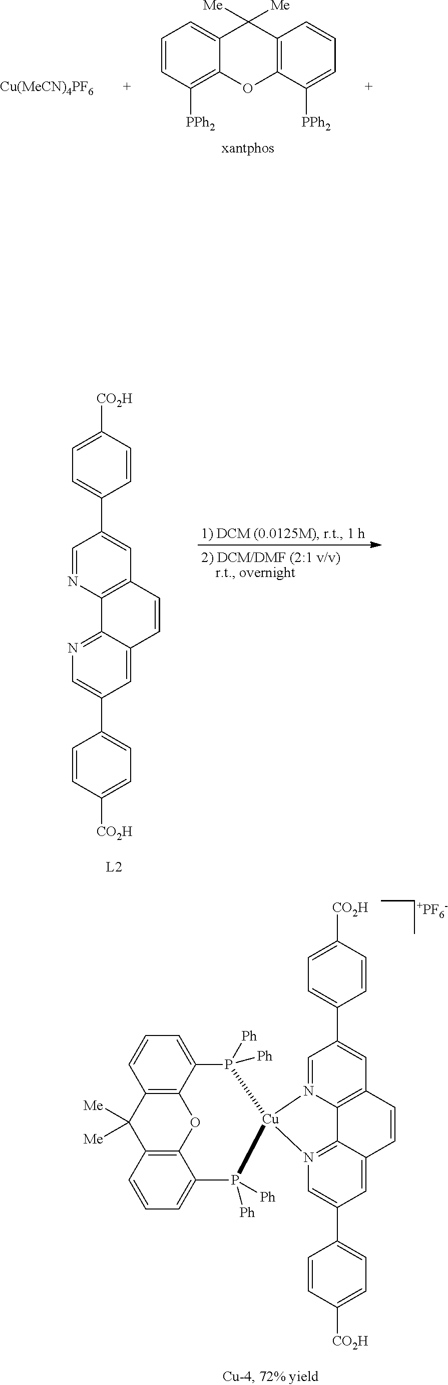

Cu-4. To a 50-mL flame-dried Schlenk flask cooled under N₂, Cu(MeCN)₄PF₆ (93 mg, 0.25 mmol), xantphos (146 mg, 0.25 mmol, 1.0 equiv.), and dichloromethane (20 mL) were added sequentially. After stirring at room temperature for 1 hour, L2 (105 mg, 0.25 mmol, 1.0 equiv.) in 15 mL of mixed solvents of dichloromethane and dimethylformamide (2:1 v/v) was added in dropwise. Then, the whole solution was stirred at room temperature overnight. After the reaction was completed, dichloromethane was removed in vacuo. The residue was diffused into diethyl ether (80 mL) dropwise and the resulted orange solid (216 mg, 0.18 mmol, 72% yield) was collected via filtration, washed by diethyl ether for several times and dried in vacuo.

¹H NMR (DMSO-d₆, 400 MHz): δ 13.2 (brs, 2H), 9.12 (s, 2H), 8.70 (s, 2H), 8.30 (s, 2H), 8.12 (d, J=8.4 Hz, 4H), 7.89 (s, 2H), 7.60 (s, 2H), 7.42-6.83 (m, 24H), 6.58 (d, J=6.9 Hz, 2H), 1.69 (s, 6H).

³¹P NMR (DMSO-d₆, 162 MHz): δ −11.4, −135.4-−153.0 (m, 1P).

FT-IR (film): 2985, 1705, 1226, 842, 743, 693 cm⁻¹.

HRMS (ESI) calculated for [C₆₅H₄₈CuN₂O₅P₂]⁺ requires m/z 1061.2329, found m/z 1061.2329.

### Preparation and Characterization of UiO-69 MOF Materials:

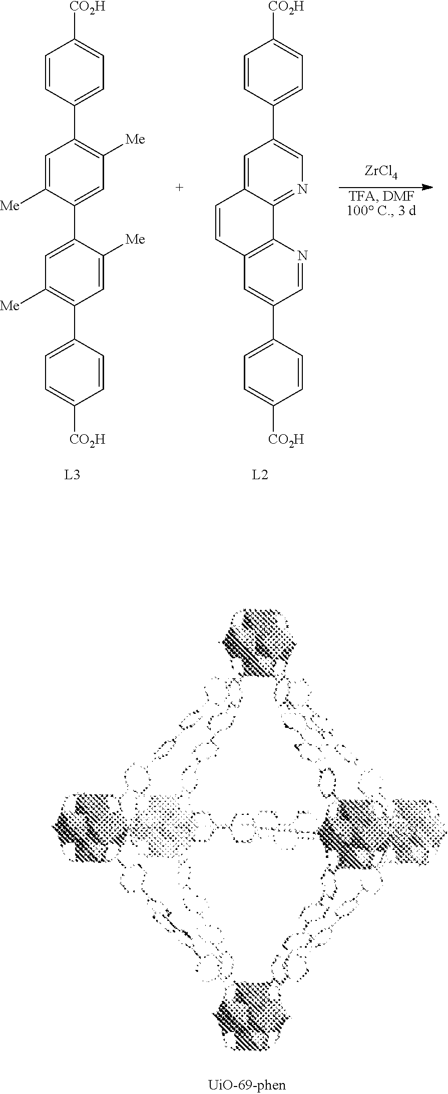 [SMILES: Cc1cc(-c2cc(C)c(-c3ccc(C(=O)O)cc3)cc2C)c(C)cc1-c1ccc(C(=O)O)cc1 ; O=C(O)c1ccc(-c2cnc3c(ccc4cc(-c5ccc(C(=O)O)cc5)cnc43)c2)cc1 ; "ZrCl4" ; "TFA, DMF100° C., 3 d"]

ZrCl₄ (179 mg, 0.77 mmol), 4,4′-(1,10-phenanthroline-3,8-diyl)dibenzoic acid (L2) (50 mg, 0.12 mmol), terphenyl dicarboxylic acid (L3) (270 mg, 0.60 mmol), and trifluoroacetic acid (0.4 mL) were ultrasonically dispersed in dimethylformamide (34 mL). The resulting solution was heated to 100° C. in a 50-mL glass tube for 3 days. After cooling to room temperature, the supernatant was decanted and the solid was washed five times with dimethylformamide and acetone. UiO-69-phen was then collected by filtration and dried in air to give a white solid (389 mg, 60% yield based on linkers). The product (2 mg) was digested in a mixture of a saturated K₃PO₄/D₂O solution and DMSO-d₆ (0.6 mL, 1:2 v/v) and subjected to ¹H NMR analysis. The ratio of L2 and L3 was found to be 1:5. The chemical formula of UiO-69-phen was determined to be Zr₆O₄(OH)₄(L2)(L3)₅-4.0DMF based on NMR digestion data and TGA.

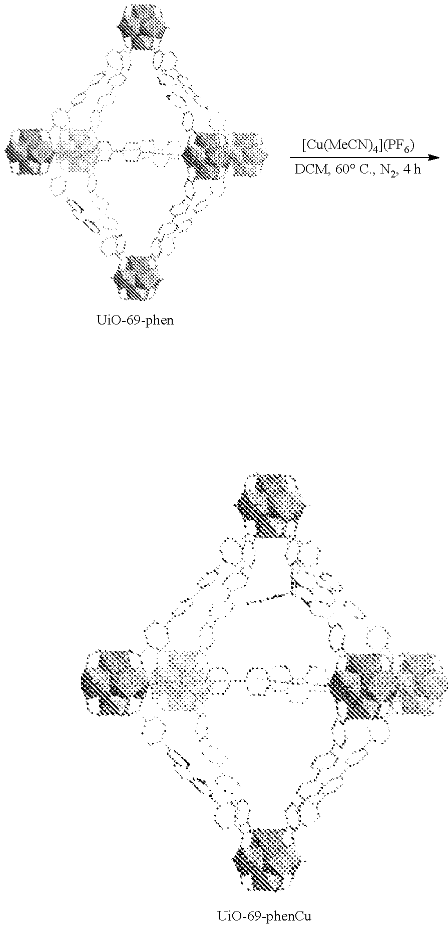

UiO-69-phen (30.0 mg, 5.6 μmol based on L2) was immersed in a 2-mL dichloromethane solution of [Cu (MeCN)₄](PF₆) (10.4 mg, 28 μmol) and stirred under N₂ atmosphere at 60° C. for 4 hours. After cooling to room temperature, the supernatant was decanted and the solid was washed five times with dimethylformamide and acetone. UiO-69-phenCu was then collected by filtration and dried in air to give a green solid (22.7 mg, 93% yield). ICP-AES analysis indicated a Zr/Cu ratio of 6:1. The chemical formula of UiO-69-phenCu was determined to be {Zr₆O₄ (OH)₄(L2)(L3)₅[Cu(CH₃CN)₂PF₆]}·0.4.4DMF based on ICP-AES and TGA.

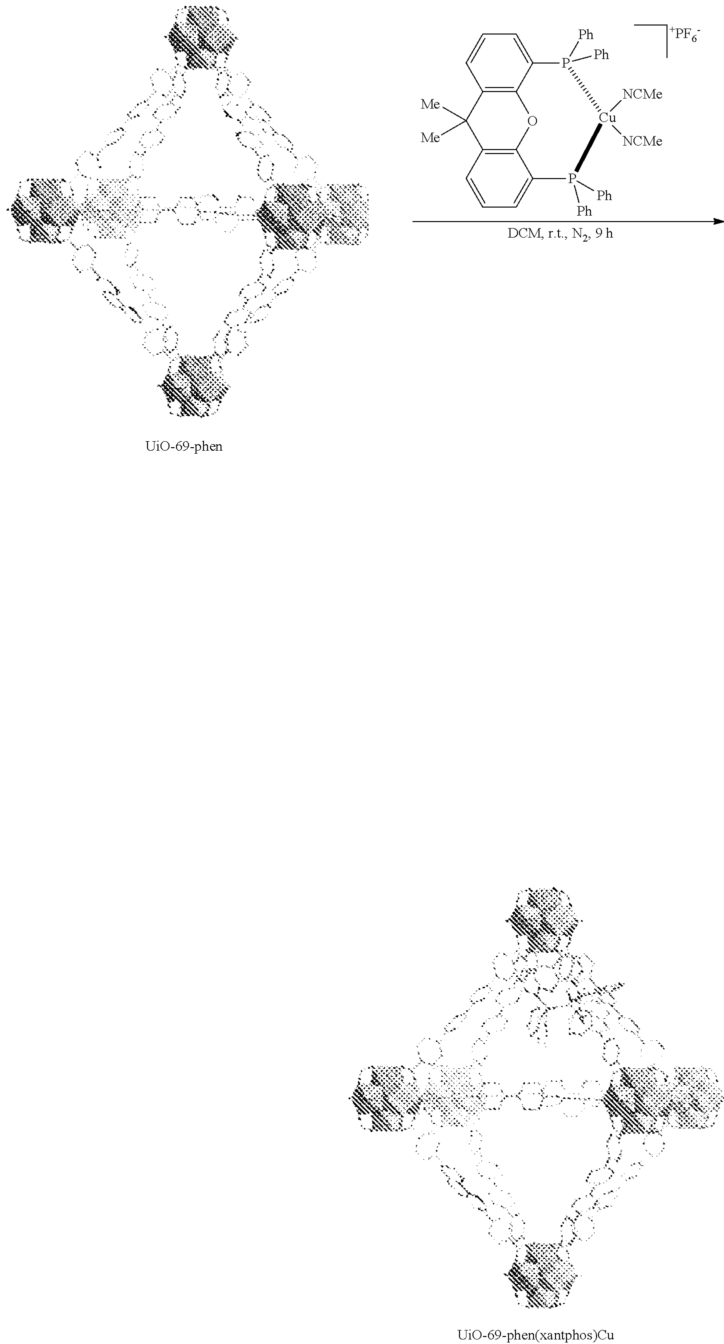

UiO-69-phen (30.0 mg, 5.6 mol based on L2) was immersed in a 2-mL dichloromethane solution of [Cu(xantphos)(MeCN)₂](PF₆) (24.3 mg, 28 mol) and stirred under N₂ atmosphere at room temperature for 9 hours. After post-synthetic metalation, the supernatant was decanted and the solid was washed five times with DMF and acetone. UiO-69-phen(xantphos)Cu was then collected by filtration and dried in air to give a yellow solid (30.9 mg, 91% yield). ICP-AES analysis indicated a Zr/Cu ratio of 6:1. The product (2 mg) was digested in a solution of D₃PO₄/D₂O/DMSO-d₆ (1:1:5 v/v/v) and subjected to ¹H NMR analysis. The ratio of Cu-3 and L3 was found to be 1:5. The chemical formula of UiO-69-phen(xantphos)Cu was determined to be

{Zr₆O₄(OH)₄(L2)(L3)₅[Cu(xantphos)PF₆]}·4.9DMF based on NMR digestion data and TGA.

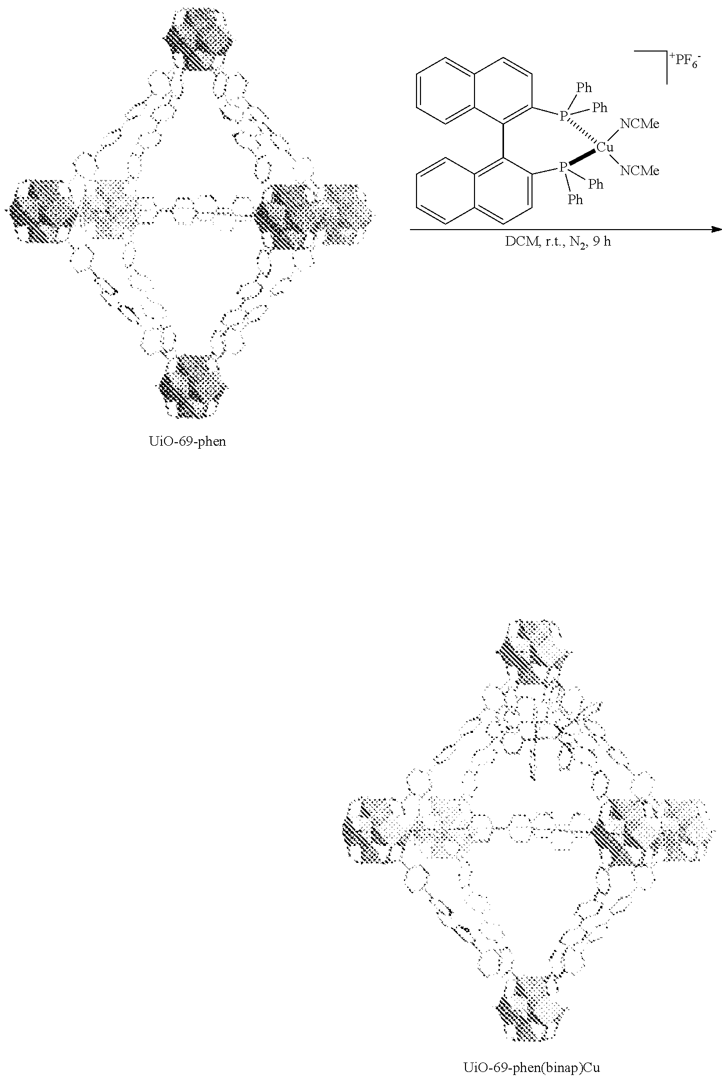

UiO-69-phen (300 mg, 56 μmol based on L2) was immersed in a 20-mL dichloromethane solution of [Cu(binap)(MeCN)₂](PF₆) (256 mg, 0.28 mmol) and stirred under N₂ atmosphere at room temperature for 9 hours. After post-synthetic metalation, the supernatant was decanted and the solid was washed five times with DMF and acetone. UiO-69-phen(binap)Cu was then collected by filtration and dried in air to give a yellow solid (312 mg, 94% yield). ICP-AES analysis indicated a Zr/Cu ratio of 6:1. The product (2 mg) was digested in a solution of D₃PO₄/D₂O/DMSO-d₆ (1:1:5 v/v/v) and subjected to ¹H NMR analysis. The ratio of Cu-2 and L3 was found to be 1:5. The chemical formula of UiO-69-phen(binap)Cu was determined to be {Zr₆O₄(OH)₄(L2)(L3)₅[Cu(binap)PF₆]}·6.0DMF based on NMR digestion data and TGA.

Based on TGA (FIG. 9), the molecular weight of as-prepared UiO-69-phen(binap)Cu was determined to be 5926. In another word, 1.0 mmol of the copper catalyst can be obtained in 5926 mg of UiO-69-phen(binap)Cu. To run a catalytic reaction (on a 0.2-mmol scale) with 0.2 mol % of the copper catalyst: The amount of UiO-69-phen(binap)Cu=5926 mg x 0.2×0.002=2.4 mg

### Synthesis of Exocyclic Arylidenes:

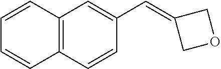 [SMILES: C(=C1COC1)c1ccc2ccccc2c1]

3-(Naphthalen-2-ylmethylene)oxetane. The title compound was synthesized according to the literature reported method² using the corresponding phosphonium bromide (9.7 g, 20 mmol, 1.0 equiv.), oxetan-3-one (1.4 mL, 24 mmol, 1.2 equiv.), ^{t}BuOK (2.2 g, 20 mmol, 1.0 equiv.), and tetrahydrofuran (200 mL). After the completion of the reaction, the crude mixture was purified by flash column chromatography using hexanes/ethyl acetate (10:1 v/v) as the eluent to give 2.7 g (13.6 mmol, 68% yield) of the title compound as a white solid.

¹H NMR (CDCl₃, 400 MHz): δ 7.84-7.72 (m, 3H), 7.52-7.40 (m, 3H), 7.10 (dd, J=8.6, 1.8 Hz, 1H), 6.31-6.20 (m, 1H), 5.72-5.63 (m, 2H), 5.48-5.39 (m, 2H).

¹³C NMR (CDCl₃, 100 MHz): δ 137.4, 133.50, 133.49, 132.2, 128.4, 127.9, 127.6, 126.4, 126.3, 126.0, 124.6, 119.8, 80.5, 80.3.

HRMS (EI) calculated for [C₁₄H₁₂O]⁺ requires m/z 196.0883, found m/z 196.0884.

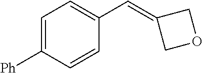 [SMILES: C(=C1COC1)c1ccc(-c2ccccc2)cc1]

3-([1,1′-Biphenyl]-4-ylmethylene)oxetane. The title compound was synthesized according to the literature reported method² using the corresponding phosphonium bromide (1.2 g, 2.0 mmol, 1.0 equiv.), oxetan-3-one (180 μL, 3.0 mmol, 1.5 equiv.), ^{t}BuOK (0.224 g, 2.0 mmol, 1.0 equiv.), and tetrahydrofuran (20 mL). After the completion of the reaction, the crude mixture was purified by flash column chromatography using hexanes/ethyl acetate (10:1 v/v) as the eluent to give 0.305 g (1.38 mmol, 69% yield) of the title compound as a white solid.

¹H NMR (CDCl₃, 400 MHz): δ 7.62-7.53 (m, 4H), 7.47-7.40 (m, 2H), 7.37-7.31 (m, 1H), 7.07 (d, J=8.4 Hz, 2H), 6.18-6.08 (m, 1H), 5.66-5.55 (m, 2H), 5.45-5.35 (m, 2H)

¹³C NMR (CDCl₃, 100 MHz): δ 140.5, 139.6, 137.2, 135.0, 128.8, 127.5, 127.4, 126.9, 119.3, 80.40, 80.27.

HRMS (EI) calculated for [C₁₆H₁₄O]⁺ requires m/z 222.1039, found m/z 222.1041.

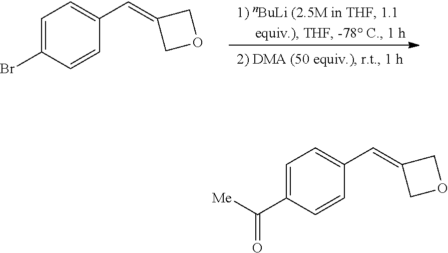 [SMILES: Brc1ccc(C=C2COC2)cc1 ; CC(=O)c1ccc(C=C2COC2)cc1]

To a 25-mL flame-dried Schlenk flask cooled under N₂, 3-(4-bromobenzylidene)oxetane (222 mg, 1.0 mmol, 1.0 equiv.) was added to 4 mL of tetrahydrofuran (0.25 M). After cooled to −78° C., ⁿBuLi (2.5 M in THF, 440 μL, 1.1 equiv.) was injected dropwise. The resulting solution was stirred at −78° C. for 1 hour and then dimethylacetamide (50 mmol, 4.6 mL) was added. The system was slowly warmed to room temperature and stirred at ambient temperature for another 1 hour. The reaction was then quenched by saturated aqueous NH₄Cl solution, extracted with ethyl acetate (3×30 mL). The combined organic layers were washed by water, brine, dried over anhydrous sodium sulfate, and concentrated in vacuo. The residue was purified by flash column chromatography using hexanes/ethyl acetate (7:1 v/v) as the eluent to give 58 mg (0.31 mmol, 31% yield) of 1-(4-(oxetan-3-ylidenemethyl)phenyl)ethan-1-one as a colorless oil.

¹H NMR (CDCl₃, 400 MHz): δ 7.92 (d, J=8.4 Hz, 2H), 7.07 (d, J=8.4 Hz, 2H), 6.19-6.14 (m, 1H), 5.62-5.56 (m, 2H), 5.44-5.37 (m, 2H), 2.59 (s, 3H).

¹³C NMR (CDCl₃, 100 MHz): δ 197.4, 140.8, 140.5, 135.3, 128.9, 127.1, 119.0, 80.3, 80.1, 26.5.

HRMS (EI) calculated for [C₁₂H₁₂O₂]⁺ requires m/z 188.0832, found m/z 188.0835.

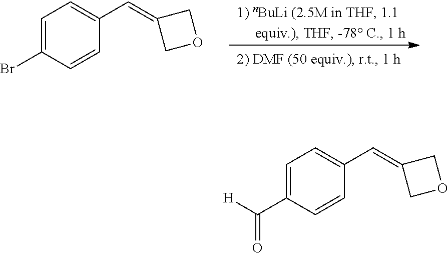 [SMILES: Brc1ccc(C=C2COC2)cc1 ; O=Cc1ccc(C=C2COC2)cc1]

To a 25-mL flame-dried Schlenk flask cooled under N₂, 3-(4-bromobenzylidene)oxetane (222 mg, 1.0 mmol, 1.0 equiv.) was added to 4 mL of tetrahydrofuran (0.25 M). After cooled to −78° C., ⁿBuLi (2.5 M in THF, 440 μL, 1.1 equiv.) was injected dropwise. The resulting solution was stirred at −78° C. for 1 hour and then dimethylformamide (50 mmol, 3.9 mL) was added. The system was slowly warmed to room temperature and stirred at ambient temperature for another 1 hour. The reaction was then quenched by saturated aqueous NH₄Cl solution, extracted with ethyl acetate (3×30 mL). The combined organic layers were washed by water, brine, dried over anhydrous sodium sulfate, and concentrated in vacuo. The residue was purified by flash column chromatography using hexanes/ethyl acetate (7:1 v/v) as the eluent to give 89 mg (0.51 mmol, 51% yield) of 4-(oxetan-3-ylidenemethyl)benzaldehyde as a colorless oil.

¹H NMR (CDCl₃, 400 MHz): δ 9.98 (s, 1H), 7.84 (d, J=8.4 Hz, 2H), 7.14 (d, J=8.4 Hz, 2H), 6.22-6.16 (m, 1H), 5.64-5.57 (m, 2H), 5.45-5.38 (m, 2H).

¹³C NMR (CDCl₃, 100 MHz): δ 191.5, 141.9, 141.7, 134.6, 130.2, 127.5, 119.0, 80.3, 80.1.

HRMS (EI) calculated for [C₁₁H₁₀O₂]⁺ requires m/z 174.0675, found m/z 174.0679.

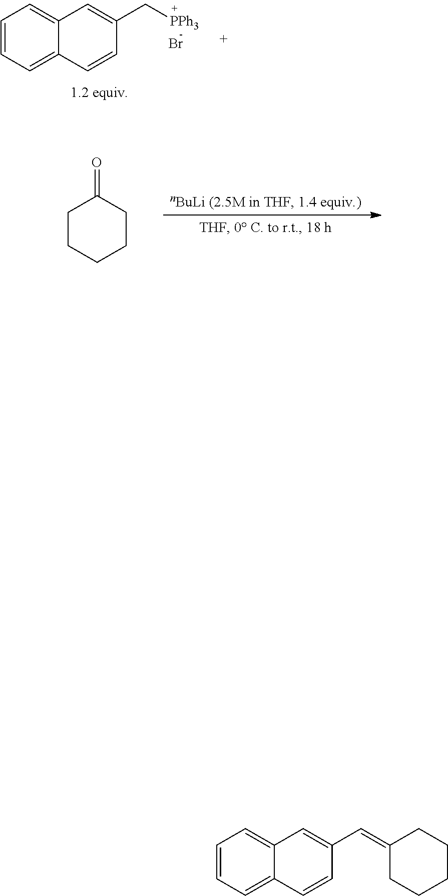

To a 50-mL flame-dried Schlenk flask cooled under N₂, the phosphonium bromide (2.9 g, 6.0 mmol, 1.2 equiv.) was added to 15 mL of tetrahydrofuran. After cooled to 0° C., ⁿBuLi (2.5 M in THF, 2.8 mL, 1.4 equiv.) was injected dropwise. The resulting solution was stirring at 0° C. for 1 hour and then cyclohexanone (5.0 mmol, 518 μL, 1.0 equiv.) in 5 mL of tetrahydrofuran was added dropwise. The system was warmed to room temperature and stirred at ambient temperature for another 18 hour. The reaction was then quenched by saturated aqueous NH₄Cl solution and extracted with ethyl acetate (3×40 mL). The combined organic layers were washed by water, brine, dried over anhydrous sodium sulfate, and concentrated in vacuo. The residue was purified by flash column chromatography using hexanes as the eluent to give 0.697 g (3.15 mmol, 63% yield) of 2-(cyclohexylidenemethyl)naphthalene as a colorless oil.

¹H NMR (CDCl₃, 400 MHz): δ 7.83-7.71 (m, 3H), 7.65-7.60 (m, 1H), 7.48-7.38 (m, 2H), 7.38-7.32 (m, 1H), 6.37 (s, 1H), 2.49-2.40 (m, 2H), 2.35-2.26 (m, 2H), 1.73-1.54 (m, 6H).

¹³C NMR (CDCl₃, 100 MHz): δ 144.0, 135.9, 133.4, 131.8, 127.8, 127.7, 127.5, 127.4, 127.1, 125.9, 125.3, 122.0, 37.7, 29.6, 28.6, 27.9, 26.7.

HRMS (EI) calculated for [C₁₇H₁₈]⁺ requires m/z 222.1403, found m/z 222.1406.

### Photoinduced Intermolecular Crossed [2+2] Cycloadditions:

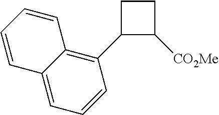 [SMILES: COC(=O)C1CCC1c1cccc2ccccc12]

Trans-methyl 2-(naphthalen-1-yl)cyclobutane-1-carboxylate (1). The title compound was synthesized according to the General Procedure A using 1-vinylnaphthalene (30.7 mg, 0.2 mmol) and methyl acrylate (91 μL, 1.0 mmol). The crude mixture was purified by flash column chromatography using hexanes/ethyl acetate (20:1 v/v) as the eluent to give 35.2 mg (0.146 mmol, 73% yield, >10:1 d.r.) of the title compound as a colorless oil.

¹H NMR (CDCl₃, 400 MHz): δ 7.96-7.92 (m, 1H), 7.86-7.82 (m, 1H), 7.74-7.70 (m, 1H), 7.51-7.44 (m, 4H), 4.44 (q, J=9.2 Hz, 1H), 3.69 (s, 3H), 3.57-3.48 (m, 1H), 2.63-2.52 (m, 1H), 2.49-2.38 (m, 1H), 2.27-2.07 (m, 2H).

¹³C NMR (CDCl₃, 100 MHz): δ 174.9, 139.0, 133.7, 131.3, 128.7, 127.0, 125.8, 125.6, 125.4, 123.9, 122.6, 51.8, 43.1, 41.0, 27.0, 22.0.

HRMS (EI) calculated for [C₁₆H₁₆O₂]⁺ requires m/z 240.1145, found m/z 240.1148.

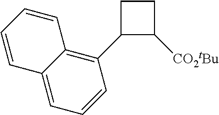 [SMILES: CC(C)(C)OC(=O)C1CCC1c1cccc2ccccc12]

Trans-tert-butyl 2-(naphthalen-1-yl)cyclobutane-1-carboxylate (2). The title compound was synthesized according to the General Procedure A using 1-vinylnaphthalene (30.9 mg, 0.2 mmol) and tert-butyl acrylate (145 ptL, 1.0 mmol). The crude mixture was purified by flash column chromatography using hexanes/ethyl acetate (20:1 v/v) as the eluent to give 46.1 mg (0.164 mmol, 80% yield, >10:1 d.r.) of the title compound as a colorless oil.

¹H NMR (CDCl₃, 400 MHz): δ 8.01-7.96 (m, 1H), 7.86-7.82 (m, 1H), 7.74-7.70 (m, 1H), 7.50-7.44 (m, 4H), 4.38 (q, J=9.2 Hz, 1H), 3.43-3.33 (m, 1H), 2.58-2.49 (m, 1H), 2.42-2.32 (m, 1H), 2.25-2.06 (m, 2H), 1.44 (s, 9H).

¹³C NMR (CDCl₃, 100 MHz): δ 173.9, 139.5, 133.7, 131.4, 128.6, 126.8, 125.7, 125.5, 125.4, 124.1, 122.7, 80.3, 44.8, 40.8, 28.1, 26.6, 21.9.

HRMS (EI) calculated for [C₁₉H₂₂O₂]⁺ requires m/z 282.1614, found m/z 282.1624.

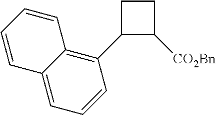 [SMILES: O=C(OCc1ccccc1)C1CCC1c1cccc2ccccc12]

Trans-benzyl 2-(naphthalen-1-yl)cyclobutane-1-carboxylate (3). The title compound was synthesized according to the General Procedure A using 1-vinylnaphthalene (30.9 mg, 0.2 mmol) and benzyl acrylate (162 mg, 1.0 mmol). The crude mixture was purified by flash column chromatography using hexanes/ethyl acetate (20:1 v/v) as the eluent to give 46.4 mg (0.146 mmol, 73% yield, >10:1 d.r.) of the title compound as a colorless oil.

¹H NMR (CDCl₃, 400 MHz): δ 7.97-7.91 (m, 1H), 7.88-7.82 (m, 1H), 7.76-7.68 (m, 1H), 7.51-7.43 (m, 4H), 7.35-7.26 (m, 5H), 5.23-5.06 (m, 2H), 4.56-4.38 (m, 1H), 3.64-3.47 (m, 1H), 2.65-2.39 (m, 2H), 2.29-2.10 (m, 2H).

¹³C NMR (CDCl₃, 100 MHz): δ 174.3, 139.0, 136.0, 133.7, 131.3, 128.7, 128.5, 128.1, 128.0, 127.0, 125.8, 125.6, 125.4, 123.9, 122.7, 66.3, 43.5, 40.9, 26.9, 22.0.

HRMS (EI) calculated for [C₂₂H₂₀O₂]⁺ requires m/z 316.1458, found m/z 316.1467.

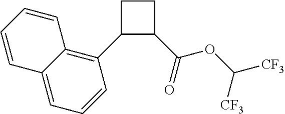 [SMILES: O=C(OC(C(F)(F)F)C(F)(F)F)C1CCC1c1cccc2ccccc12]

Trans-3,3,3-trifluoro-2-(trifluoromethyl)propyl-2-(naphthalen-1-yl)cyclobutane-1-carboxylate (4). The title compound was synthesized according to the General Procedure A using 1-vinylnaphthalene (30.8 mg, 0.2 mmol) and 1,1,1,3,3,3-hexafluoropropan-2-yl acrylate (222 mg, 1.0 mmol). The crude mixture was purified by flash column chromatography using hexanes/ethyl acetate (20:1 v/v) as the eluent to give 45.0 mg (0.120 mmol, 60% yield, >10:1 d.r.) of the title compound as a colorless oil.

¹H NMR (CDCl₃, 400 MHz): δ 7.94-7.83 (m, 2H), 7.75 (d, J=8.0 Hz, 1H), 7.53-7.44 (m, 3H), 7.43-7.38 (m, 1H), 5.90-5.72 (m, 1H), 4.52 (q, J=9.2 Hz, 1H), 3.77-3.64 (m, 1H), 2.75-2.61 (m, 1H), 2.54-2.32 (m, 2H), 2.29-2.16 (m, 1H).

¹⁹F NMR (376 MHz, CDCl₃): δ −73.3.

¹³C NMR (CDCl₃, 100 MHz): δ 171.1, 138.0, 133.8, 131.0, 128.8, 127.4, 126.0, 125.8, 125.4, 123.6, 122.5, 120.4 (q, J=272.2 Hz), 66.6 (p, J=34.7 Hz), 42.2, 40.7, 27.1, 22.2.

HRMS (EI) calculated for [C₁₈H₁₄F₆O₂]⁺ requires m/z 376.0893, found m/z 376.0893.

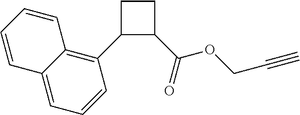 [SMILES: C#CCOC(=O)C1CCC1c1cccc2ccccc12]

Trans-prop-2-yn-1-yl 2-(naphthalen-1-yl)cyclobutane-1-carboxylate (5). The title compound was synthesized according to the General Procedure A using 1-vinylnaphthalene (30.8 mg, 0.2 mmol) and prop-2-yn-1-yl acrylate (110 mg, 1.0 mmol). The crude mixture was purified by flash column chromatography using hexanes/ethyl acetate (20:1 v/v) as the eluent to give 38.1 mg (0.144 mmol, 72% yield, >10:1 d.r.) of the title compound as a colorless oil.

¹H NMR (CDCl₃, 400 MHz): δ 7.98-7.91 (m, 1H), 7.87-7.82 (m, 1H), 7.76-7.70 (m, 1H), 7.52-7.42 (m, 4H), 4.70 (qd, J=15.6, 2.4 Hz, 2H), 4.48 (q, J=9.2 Hz, 1H), 3.63-3.49 (m, 1H), 2.65-2.54 (m, 1H), 2.51-2.38 (m, 2H), 2.31-2.10 (m, 2H).

¹³C NMR (CDCl₃, 100 MHz): δ 173.6, 138.8, 133.8, 131.2, 128.7, 127.0, 125.9, 125.6, 125.4, 123.8, 122.6, 77.7, 74.9, 52.1, 43.0, 40.8, 27.0, 22.0.

HRMS (EI) calculated for [C₁₈H₁₆O₂]⁺ requires m/z 264.1145, found m/z 264.1153.

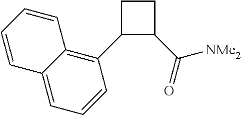 [SMILES: CN(C)C(=O)C1CCC1c1cccc2ccccc12]

Trans-N,N-dimethyl-2-(naphthalen-1-yl)cyclobutane-1-carboxamide (6). The title compound was synthesized according to the General Procedure A using 1-vinylnaphthalene (30.8 mg, 0.2 mmol) and N,N-dimethylacrylamide (110 μL, 1.0 mmol). The crude mixture was purified by flash column chromatography using hexanes/ethyl acetate (3:1 v/v) as the eluent to give 37.0 mg (0.146 mmol, 73% yield, >10:1 d.r.) of the title compound as a colorless oil.

¹H NMR (CDCl₃, 400 MHz): δ 8.11-8.05 (m, 1H), 7.86-7.80 (m, 1H), 7.71 (d, J=7.6 Hz, 1H), 7.52-7.38 (m, 4H), 4.70-4.57 (m, 1H), 3.59-3.47 (m, 1H), 2.94 (s, 3H), 2.76 (s, 3H), 2.54-2.45 (m, 1H), 2.41-2.17 (m, 3H).

¹³C NMR (CDCl₃, 100 MHz): δ 173.6, 140.1, 133.8, 131.5, 128.5, 126.8, 125.7, 125.6, 125.4, 124.2, 122.6, 43.7, 39.2, 36.7, 35.6, 25.3, 22.3.

HRMS (EI) calculated for [C₁₇H₁₉NO]⁺ requires m/z 253.1461, found m/z 253.1471.

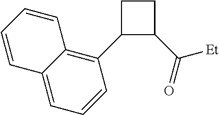 [SMILES: CCC(=O)C1CCC1c1cccc2ccccc12]

1-(2-(Naphthalen-1-yl)cyclobutyl)propan-1-one (7). The title compound was synthesized according to the General Procedure A using 1-vinylnaphthalene (30.8 mg, 0.2 mmol) and pent-1-en-3-one (89 μL, 1.0 mmol). The crude mixture was purified by flash column chromatography using hexanes/ethyl acetate (15:1 v/v) as the eluent to give 31.9 mg (0.134 mmol, 67% yield, 6.0:1 d.r.) of the title compound as a colorless oil.

¹H NMR (CDCl₃, 400 MHz): δ 8.01-7.91 (m, 1H), 7.89-7.81 (m, 1H), 7.76-7.70 (m, 1H), 7.52-7.42 (m, 4H), 4.48-4.35 (m, 1H), 3.69-3.57 (m, 1H), 2.53-2.29 (m, 4H), 2.23-2.10 (m, 2H), 1.03 (t, J=7.2 Hz, 3H).

¹³C NMR (CDCl₃, 100 MHz): δ 211.7, 139.5, 133.8, 131.3, 128.7, 126.9, 125.9, 125.7, 125.4, 123.8, 122.5, 50.4, 39.5, 34.6, 26.1, 21.5, 7.6.

HRMS (EI) calculated for [C₁₇H₁₈O₄]⁺ requires m/z 238.1352, found m/z 238.1354.

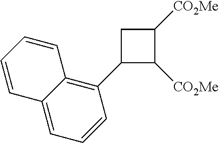 [SMILES: COC(=O)C1CC(c2cccc3ccccc23)C1C(=O)OC]

Dimethyl 3-(naphthalen-1-yl)cyclobutane-1,2-dicarboxylate (8). The title compound was synthesized according to the General Procedure A using 1-vinylnaphthalene (30.8 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (10:1 v/v) as the eluent to give 52.0 mg (0.174 mmol, 87% yield, 2.0:1 d.r.) of the title compound as a colorless oil.

¹H NMR (CDCl₃, 400 MHz): δ 7.98 (d, J=8.4 Hz, 0.34H), 7.91-7.81 (m, 1.66H), 7.77-7.71 (m, 1H), 7.55-7.42 (m, 4H), 4.83 (q, J=9.2 Hz, 0.34H), 4.31 (q, J=9.6 Hz, 0.66H), 4.13-3.95 (m, 0.34H), 3.84-3.76 (m, 1.68H), 3.75-3.62 (m, 4.30H), 3.60-3.49 (m, 0.66H), 3.16-3.06 (m, 0.34H), 2.93-2.83 (m, 1.68H), 2.73-2.64 (m, 0.34H), 2.33 (q, J=10.4 Hz, 0.66H).

¹³C NMR (CDCl₃, 100 MHz): δ 174.8, 173.5, 173.2, 171.7, 137.5, 134.9, 133.7, 133.5, 131.6, 131.2, 128.8, 128.5, 127.6, 127.4, 126.0, 125.7, 125.6, 125.5, 125.2, 123.7, 123.5, 123.4, 123.0, 52.17, 52.14, 52.07, 51.0, 47.3, 45.7, 37.3, 37.1, 36.7, 36.5, 30.7, 26.5.

HRMS (EI) calculated for [C₁₈H₁₈O₄]⁺ requires m/z 298.1200, found m/z 298.1202.

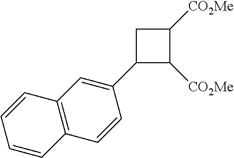 [SMILES: COC(=O)C1CC(c2ccc3ccccc3c2)C1C(=O)OC]

Dimethyl 3-(naphthalen-2-yl)cyclobutane-1,2-dicarboxylate (9). The title compound was synthesized according to the General Procedure A using 2-vinylnaphthalene (30.9 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (20:1 v/v) as the eluent to give 48.9 mg (0.164 mmol, 82% yield, 1.5:1 d.r.) of the title compound as a colorless oil.

¹H NMR (CDCl₃, 400 MHz): δ 7.86-7.73 (m, 3H), 7.71-7.64 (m, 1H), 7.51-7.38 (m, 2.60H), 7.33 (dd, J=8.4, 1.6 Hz, 0.40H), 4.21-4.08 (m, 0.40H), 3.98-3.86 (m, 0.40H), 3.84-3.68 (m, 5.80H), 3.65-3.57 (m, 0.60H), 3.51-3.40 (m, 0.60H), 3.19 (s, 1.20H), 2.90-2.79 (m, 0.40H), 2.77-2.63 (m, 1H), 2.50-2.38 (m, 0.60H).

¹³C NMR (CDCl₃, 100 MHz): δ 174.6, 173.6, 173.1, 171.6, 139.5, 137.3, 133.3, 133.2, 132.4, 128.3, 127.82, 127.78, 127.7, 127.6, 127.5, 126.2, 126.0, 125.8, 125.74, 125.66, 125.0, 124.9, 52.08, 52.05, 51.4, 47.5, 46.5, 40.0, 39.8, 36.8, 36.6, 29.4, 27.7.

HRMS (EI) calculated for [C₁₈H₁₈O₄]⁺ requires m/z 298.1200, found m/z 298.1208.

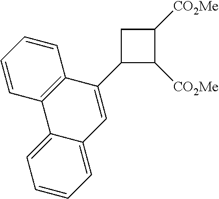 [SMILES: COC(=O)C1CC(c2cc3ccccc3c3ccccc23)C1C(=O)OC]

Dimethyl 3-(phenanthren-9-yl)cyclobutane-1,2-dicarboxylate (10). The title compound was synthesized according to the General Procedure A using 9-vinylphenanthrene (40.8 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (20:1 v/v) as the eluent to give 45.3 mg (0.130 mmol, 65% yield, 1.5:1 d.r.) of the title compound as a colorless oil.

¹H NMR (CDCl₃, 400 MHz): δ 8.76-8.67 (m, 1H), 8.67-8.60 (m, 1H), 8.05-7.98 (m, 0.40H), 7.94-7.85 (m, 1.60H), 7.73 (s, 0.60H), 7.69-7.54 (m, 4.40H), 4.92-4.73 (m, 0.40H), 4.37-4.25 (m, 0.60H), 4.11-4.04 (m, 0.40H), 3.96-3.88 (m, 0.60H), 3.82 (s, 1.20H), 3.76-3.71 (m, 3.60H), 3.70-3.65 (m, 0.40H), 3.64-3.55 (m, 0.60H), 3.31-3.19 (m, 0.40H), 2.97-2.88 (m, 0.60H), 2.86 (s, 1.20H), 2.77-2.67 (m, 0.40H), 2.41-2.27 (m, 0.60H).

¹³C NMR (CDCl₃, 100 MHz): δ 174.8, 173.5, 173.3, 171.7, 135.7, 133.1, 131.5, 131.3, 130.7, 130.4, 130.3, 129.8, 129.7, 129.5, 128.7, 128.6, 126.8, 126.7, 126.60, 126.57, 126.55, 126.5, 126.34, 126.31, 124.7, 124.5, 124.1, 123.9, 123.3, 122.9, 122.39, 122.36, 52.21, 52.19, 52.1, 51.0, 47.4, 45.2, 37.6, 37.2, 37.1, 36.81, 30.78, 26.3.

HRMS (EI) calculated for [C₂₂H₂₀O₄]⁺ requires m/z 348.1356, found m/z 348.1365.

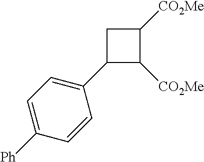 [SMILES: COC(=O)C1CC(c2ccc(-c3ccccc3)cc2)C1C(=O)OC]

Dimethyl 3-([1,1′-biphenyl]-4-yl)cyclobutane-1,2-dicarboxylate (11). The title compound was synthesized according to the General Procedure A using 4-vinyl-1,1′-biphenyl (36.0 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (20:1 v/v) as the eluent to give 51.9 mg (0.160 mmol, 80% yield, 1.5:1 d.r.) of the title compound as a colorless oil.

¹H NMR (CDCl₃, 400 MHz): δ 7.61-7.52 (m, 4H), 7.46-7.40 (m, 2H), 7.38-7.27 (m, 3H), 4.08-3.97 (m, 0.40H), 3.92-3.84 (m, 0.40H), 3.79-3.71 (m, 5.20H), 3.70-3.63 (m, 0.60H), 3.58-3.50 (m, 0.60H), 3.47-3.37 (m, 0.60H), 3.29 (s, 1.20H), 2.79-2.59 (m, 1.40H), 2.43-2.31 (m, 0.60H).

¹³C NMR (CDCl₃, 100 MHz): δ 174.6, 173.6, 173.1, 171.6, 141.2, 140.8, 140.6, 139.8, 139.7, 138.9, 128.7, 127.8, 127.24, 127.21, 127.02, 127.00, 126.91, 126.88, 52.09, 52.06, 51.4, 47.6, 46.5, 39.6, 39.4, 36.8, 36.6, 29.4, 27.8.

HRMS (EI) calculated for [C₂₀H₂₀O₄]⁺ requires m/z 324.1356, found m/z 324.1365.

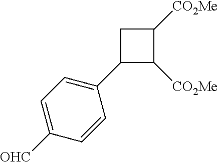 [SMILES: COC(=O)C1CC(c2ccc(C=O)cc2)C1C(=O)OC]

Dimethyl 3-(4-formylphenyl)cyclobutane-1,2-dicarboxylate (12). The title compound was synthesized according to the General Procedure A using 4-vinylbenzaldehyde (26.4 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (20:1 v/v) as the eluent to give 28.2 mg (0.102 mmol, 51% yield, 1.9:1 d.r.) of the title compound as a colorless oil.

¹H NMR (CDCl₃, 400 MHz): δ 10.00 (s, 0.65H), 9.99 (s, 0.35H), 7.91-7.78 (m, 2H), 7.50-7.42 (m, 1.35H), 7.39 (d, J=8.0 Hz, 0.65H), 4.14-4.03 (m, 0.35H), 3.95-3.86 (m, 0.35H), 3.80-3.69 (m, 5.95H), 3.57-3.38 (m, 1.30H), 3.28 (s, 1.05H), 2.80-2.59 (m, 1.35H), 2.41-2.30 (m, 0.65H).

¹³C NMR (CDCl₃, 100 MHz): δ 191.84, 191.83, 174.2, 173.4, 172.7, 171.2, 149.1, 147.1, 135.1, 130.1, 129.7, 128.0, 127.2, 127.1, 52.24, 52.20, 52.17, 51.5, 47.2, 46.4, 39.9, 39.5, 36.8, 36.6, 29.0, 27.5.

HRMS (EI) calculated for [C₁₅H₁₆O₅]⁺ requires m/z 276.0992, found m/z 276.1003.

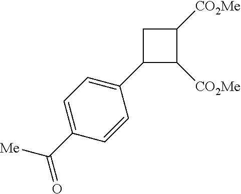 [SMILES: COC(=O)C1CC(c2ccc(C(C)=O)cc2)C1C(=O)OC]

Dimethyl 3-(4-acetylphenyl)cyclobutane-1,2-dicarboxylate (13). The title compound was synthesized according to the General Procedure A using 1-(4-vinylphenyl)ethan-1-one (32.4 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (20:1 v/v) as the eluent to give 31.9 mg (0.110 mmol, 55% yield, 1.9:1 d.r.) of the title compound as a colorless oil.

¹H NMR (CDCl₃, 400 MHz): δ 7.96-7.87 (m, 2H), 7.40-7.34 (m, 1.34H), 7.32 (d, J=8.3 Hz, 0.66H), 4.11-3.99 (m, 0.34H), 3.97-3.84 (m, 0.34H), 3.78-3.66 (m, 5.66H), 3.56-3.38 (m, 1.32H), 3.29 (s, 1.02H), 2.78-2.55 (m, 4.66H), 2.41-2.29 (m, 0.66H).

¹³C NMR (CDCl₃, 100 MHz): δ 197.71, 197.68, 174.3, 173.4, 172.8, 171.3, 147.5, 145.5, 135.8, 128.7, 128.4, 127.6, 126.8, 126.6, 52.20, 52.17, 52.1, 51.5, 47.3, 46.3, 39.7, 39.4, 36.8, 36.6, 29.1, 27.5, 26.60, 26.58.

HRMS (EI) calculated for [C₁₆H₁₈O₅]⁺ requires m/z 290.1149, found m/z 290.1156.

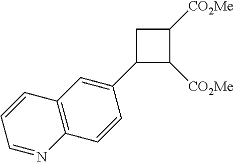 [SMILES: COC(=O)C1CC(c2ccc3ncccc3c2)C1C(=O)OC]

Dimethyl 3-(quinolin-6-yl)cyclobutane-1,2-dicarboxylate (14). The title compound was synthesized according to the General Procedure A using 6-vinylquinoline (31.1 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (6:1→3:1 v/v) as the eluent to give 31.7 mg (0.106 mmol, 53% yield, 1.4:1 d.r.) of the title compound as a colorless oil.

¹H NMR (CDCl₃, 400 MHz): δ 8.96-8.83 (m, 1H), 8.17-8.11 (m, 1H), 8.08 (d, J=8.7 Hz, 0.55H), 8.04 (d, J=8.7 Hz, 0.45H), 7.71-7.64 (m, 1.55H), 7.57 (dd, J=8.7, 2.1 Hz, 0.45H), 7.45-7.37 (m, 1H), 4.25-4.15 (m, 0.45H), 4.00-3.90 (m, 0.45H), 3.88-3.72 (m, 5.65H), 3.65-3.58 (m, 0.55H), 3.50-3.42 (m, 0.55H), 3.20 (s, 1.35H), 2.92-2.81 (m, 0.45H), 2.78-2.68 (m, 1H), 2.49-2.40 (m, 0.55H).

¹³C NMR (CDCl₃, 100 MHz): δ 174.4, 173.5, 172.9, 171.5, 150.24, 150.17, 147.4, 140.4, 138.3, 136.0, 135.9, 129.74, 129.68, 129.3, 128.6, 128.1, 128.0, 125.5, 124.8, 124.5, 121.4, 121.3, 52.18, 52.16, 52.1, 51.5, 47.5, 46.5, 39.8, 39.5, 36.8, 36.6, 29.3, 27.6.

HRMS (EI) calculated for [C₁₇H₁₇NO₄]⁺ requires m/z 299.1152, found m/z 299.1155.

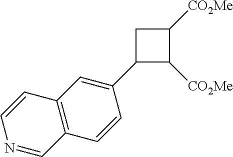 [SMILES: COC(=O)C1CC(c2ccc3cnccc3c2)C1C(=O)OC]

Dimethyl 3-(isoquinolin-6-yl)cyclobutane-1,2-dicarboxylate (15). The title compound was synthesized according to the General Procedure A using 6-vinylisoquinoline (31.1 mg, 0.2 mmol), dimethyl fumarate (34.6 mg, 0.24 mmol), and UiO-69-phen(binap)Cu (8.9 mg, 1.5 μmol based on Cu, 0.75 mol %). The crude mixture was purified by flash column chromatography using hexanes/ethyl acetate (6:1-3:1 v/v) as the eluent to give 29.9 mg (0.100 mmol, 50% yield, 1.5:1 d.r.) of the title compound as a colorless oil.

¹H NMR (CDCl₃, 400 MHz): δ 8.95-8.81 (m, 1H), 8.19-8.01 (m, 2H), 7.72-7.63 (m, 1.60H), 7.57 (dd, J=8.8, 2.1 Hz, 0.40H), 7.45-7.35 (m, 1H), 4.26-4.14 (m, 0.40H), 4.01-3.90 (m, 0.40H), 3.88-3.72 (m, 5.80H), 3.65-3.58 (m, 0.60H), 3.52-3.42 (m, 0.60H), 3.20 (s, 1.20H), 2.89-2.81 (m, 0.40H), 2.78-2.66 (m, 1H), 2.51-2.38 (m, 0.60H).

¹³C NMR (CDCl₃, 100 MHz): δ 174.4, 173.5, 172.9, 171.5, 150.3, 150.2, 147.4, 140.4, 138.2, 136.0, 135.9, 129.73, 129.68, 129.3, 128.6, 128.1, 128.0, 125.5, 124.8, 121.4, 121.3, 52.19, 52.16, 52.1, 51.5, 47.5, 46.5, 39.8, 39.5, 36.8, 36.6, 29.3, 27.6.

HRMS (EI) calculated for [C₁₇H₁₇NO₄]⁺ requires m/z 299.1152, found m/z 299.1153.

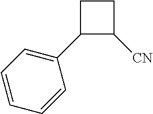 [SMILES: N#CC1CCC1c1ccccc1]

2-Phenylcyclobutane-1-carbonitrile (16). The title compound was synthesized according to the General Procedure A using styrene (41.6 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 41.5 mg (0.264 mmol, 66% yield, 1.3:1 d.r.) of the title compound as a colorless oil.

Major: ¹H NMR (CDCl₃, 400 MHz): δ 7.39-7.31 (m, 2H), 7.30-7.19 (m, 3H), 3.96-3.78 (m, 1H), 3.13-3.01 (m, 1H), 2.45-2.21 (m, 4H).

¹³C NMR (CDCl₃, 100 MHz): δ 141.2, 128.7, 127.2, 126.0, 121.3, 45.0, 29.2, 26.5, 23.1.

Minor: ¹H NMR (CDCl₃, 400 MHz): δ 7.43-7.34 (m, 2H), 7.33-7.24 (m, 3H), 3.95-3.84 (m, 1H), 3.56-3.46 (m, 1H), 2.75-2.60 (m, 1H), 2.55-2.35 (m, 2H), 2.34-2.25 (m, 1H).

¹³C NMR (CDCl₃, 100 MHz): δ 139.2, 128.6, 127.4, 127.1, 120.8, 41.7, 30.7, 25.7, 23.2.

HRMS (EI) calculated for [CiiHiiN]⁺ requires m/z 157.0886, found m/z 157.0870.

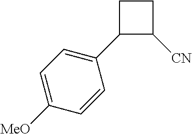 [SMILES: COc1ccc(C2CCC2C#N)cc1]

2-(4-Methoxyphenyl)cyclobutane-1-carbonitrile (17). The title compound was synthesized according to the General Procedure A using 1-methoxy-4-vinylbenzene (53.7 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 48.7 mg (0.260 mmol, 65% yield, 1.2:1 d.r.) of the title compound as a colorless oil.

Major: ¹H NMR (CDCl₃, 400 MHz): δ 7.16 (d, J=8.6 Hz, 2H), 6.88 (d, J=8.6 Hz, 2H), 3.85-3.73 (m, 4H), 3.07-2.94 (m, 1H), 2.40-2.17 (m, 4H).

¹³C NMR (CDCl₃, 100 MHz): δ 158.7, 133.3, 127.2, 121.4, 114.0, 55.3, 44.6, 29.6, 26.8, 23.0.

Minor: ¹H NMR (CDCl₃, 400 MHz): δ 7.23 (d, J=8.6 Hz, 2H), 6.91 (d, J=8.6 Hz, 2H), 3.88-3.77 (m, 4H), 3.52-3.41 (m, 1H), 2.69-2.57 (m, 1H), 2.50-2.32 (m, 2H), 2.30-2.22 (m, 1H).

¹³C NMR (CDCl₃, 100 MHz): δ 158.8, 131.4, 128.3, 121.0, 113.9, 55.2, 41.2, 30.9, 26.0, 23.0.

HRMS (EI) calculated for [C₁₂H₁₃NO]⁺ requires m/z 187.0992, found m/z 187.0980.

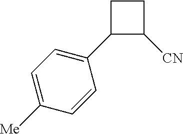 [SMILES: Cc1ccc(C2CCC2C#N)cc1]

2-(p-Tolyl)cyclobutane-1-carbonitrile (18). The title compound was synthesized according to the General Procedure A using 1-methyl-4-vinylbenzene (47.3 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 41.0 mg (0.240 mmol, 60% yield, 1.3:1 d.r.) of the title compound as a colorless oil.

Major: ¹H NMR (CDCl₃, 400 MHz): δ 7.19-7.10 (m, 4H), 3.89-3.75 (m, 1H), 3.09-2.98 (m, 1H), 2.42-2.22 (m, 7H).

¹³C NMR (CDCl₃, 100 MHz): δ 138.3, 136.9, 129.3, 125.9, 121.4, 44.8, 29.4, 26.6, 23.1, 21.0.

Minor: ¹H NMR (CDCl₃, 400 MHz): δ 7.18 (s, 4H), 3.93-3.78 (m, 1H), 3.55-3.41 (m, 1H), 2.71-2.59 (m, 1H), 2.52-2.24 (m, 6H).

¹³C NMR (CDCl₃, 100 MHz): δ 137.0, 136.2, 129.2, 127.0, 120.9, 41.5, 30.8, 25.8, 23.1, 21.1.

HRMS (EI) calculated for [C₁₂H₁₃N]⁺ requires m/z 171.1043, found m/z 171.1039.

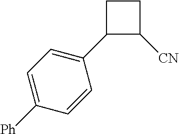 [SMILES: N#CC1CCC1c1ccc(-c2ccccc2)cc1]

2-([1,1′-Biphenyl]-4-yl)cyclobutane-1-carbonitrile (19). The title compound was synthesized according to the General Procedure A using 4-vinyl-1,1′-biphenyl (36.1 mg, 0.2 mmol) and acrylonitrile (66 μL, 1.0 mmol). The crude mixture was purified by flash column chromatography using hexanes/ethyl acetate (10:1 v/v) as the eluent to give 24.7 mg (0.106 mmol, 53% yield, 1.4:1 d.r.) of the title compound as a colorless oil.

Major: ¹H NMR (CDCl₃, 400 MHz): δ 7.60-7.54 (m, 4H), 7.48-7.40 (m, 2H), 7.38-7.28 (m, 3H), 3.98-3.82 (m, 1H), 3.18-3.03 (m, 1H), 2.49-2.22 (m, 4H).

¹³C NMR (CDCl₃, 100 MHz): δ 140.6, 140.2, 128.8, 127.39, 127.36, 127.0, 126.5, 121.3, 44.8, 29.3, 26.6, 23.2.

Minor: ¹H NMR (CDCl₃, 400 MHz): δ 7.65-7.55 (m, 4H), 7.48-7.40 (m, 2H), 7.39-7.29 (m, 3H), 4.03-3.84 (m, 1H), 3.61-3.38 (m, 1H), 2.79-2.62 (m, 1H), 2.56-2.37 (m, 2H), 2.36-2.26 (m, 1H).

¹³C NMR (CDCl₃, 100 MHz): δ 140.7, 140.2, 138.3, 128.7, 127.6, 127.3, 127.2, 127.1, 120.8, 41.5, 30.7, 25.8, 23.2.

HRMS (EI) calculated for [C₁₇H₁₅N]⁺ requires m/z 233.1199, found m/z 233.1206.

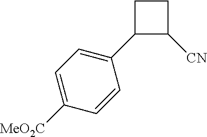 [SMILES: COC(=O)c1ccc(C2CCC2C#N)cc1]

Methyl 4-(2-cyanocyclobutyl)benzoate (20). The title compound was synthesized according to the General Procedure A using methyl 4-vinylbenzoate (32.4 mg, 0.2 mmol) and acrylonitrile (66 μL, 1.0 mmol). The crude mixture was purified by flash column chromatography using hexanes/ethyl acetate (5:1 v/v) as the eluent to give 20.7 mg (0.096 mmol, 48% yield, 1.3:1 d.r.) of the title compound as a colorless oil.

Major: ¹H NMR (CDCl₃, 400 MHz): δ 8.02 (d, J=8.4 Hz, 2H), 7.30 (d, J=8.4 Hz, 2H), 3.98-3.86 (m, 4H), 3.16-3.06 (m, 1H), 2.48-2.22 (m, 4H).

¹³C NMR (CDCl₃, 100 MHz): δ 166.7, 146.2, 130.0, 129.1, 126.0, 121.0, 52.2, 44.7, 29.0, 26.3, 23.2.

Minor: ¹H NMR (CDCl₃, 400 MHz): δ 8.06 (d, J=8.4 Hz, 2H), 7.35 (d, J=8.4 Hz, 2H), 4.01-3.88 (m, 4H), 3.60-3.51 (m, 1H), 2.75-2.64 (m, 1H), 2.58-2.40 (m, 2H), 2.36-2.27 (m, 1H).

¹³C NMR (CDCl₃, 100 MHz): δ 166.8, 144.4, 129.9, 129.2, 127.1, 120.4, 52.1, 41.5, 30.5, 25.5, 23.2.

HRMS (EI) calculated for [C₁₃H₁₃NO₂]⁺ requires m/z 215.0941, found m/z 215.0938.

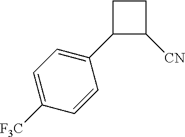 [SMILES: N#CC1CCC1c1ccc(C(F)(F)F)cc1]

2-(4-(Trifluoromethyl)phenyl)cyclobutane-1-carbonitrile (21). The title compound was synthesized according to the General Procedure A using 1-(trifluoromethyl)-4-vinylbenzene (68.9 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 52.2 mg (0.232 mmol, 58% yield, 1.3:1 d.r.) of the title compound as a colorless oil.

Major: ¹H NMR (CDCl₃, 400 MHz): δ 7.61 (d, J=8.0 Hz, 2H), 7.35 (d, J=8.0 Hz, 2H), 3.99-3.89 (m, 1H), 3.14-3.04 (m, 1H), 2.49-2.22 (m, 4H).

¹⁹F NMR (CDCl₃, 376 MHz): δ −62.5.

¹³C NMR (CDCl₃, 100 MHz): δ 145.1, 129.6 (q, J=33.0 Hz), 126.4, 125.7 (q, J=3.8 Hz), 124.0 (q, J=270.0 Hz), 120.8, 44.5, 29.0, 26.3, 23.2.

Minor: ¹H NMR (CDCl₃, 400 MHz): δ 7.64 (d, J=8.0 Hz, 2H), 7.40 (d, J=8.0 Hz, 2H), 4.01-3.90 (m, 1H), 3.61-3.51 (m, 1H), 2.75-2.63 (m, 1H), 2.58-2.42 (m, 2H), 2.36-2.28 (m, 1H).

¹⁹F NMR (CDCl₃, 376 MHz): δ −62.5.

¹³C NMR (CDCl₃, 100 MHz): δ 143.2, 129.6 (q, J=32.4 Hz), 127.5, 125.6 (q, J=3.8 Hz), 124.1 (q, J=270.0 Hz), 120.4, 41.3, 30.5, 25.6, 23.2.

HRMS (EI) calculated for [C₁₂H₁₀F₃N]⁺ requires m/z 225.0760, found m/z 225.0762.

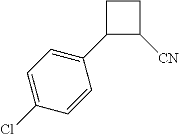 [SMILES: N#CC1CCC1c1ccc(Cl)cc1]

2-(4-Chlorophenyl)cyclobutane-1-carbonitrile (22). The title compound was synthesized according to the General Procedure A using 1-chloro-4-vinylbenzene (55.4 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 49.7 mg (0.260 mmol, 65% yield, 1.4:1 d.r.) of the title compound as a colorless oil.

Major: ¹H NMR (CDCl₃, 400 MHz): δ 7.32 (d, J=8.4 Hz, 2H), 7.16 (d, J=8.4 Hz, 2H), 3.90-3.76 (m, 1H), 3.08-2.97 (m, 1H), 2.44-2.15 (m, 4H).

¹³C NMR (CDCl₃, 100 MHz): δ 139.6, 133.0, 128.8, 127.4, 121.0, 44.4, 29.2, 26.5, 23.1.

Minor: ¹H NMR (CDCl₃, 400 MHz): δ 7.35 (d, J=8.4 Hz, 2H), 7.22 (d, J=8.4 Hz, 2H), 3.92-3.80 (m, 1H), 3.56-3.44 (m, 1H), 2.70-2.58 (m, 1H), 2.54-2.36 (m, 2H), 2.33-2.24 (m, 1H).

¹³C NMR (CDCl₃, 100 MHz): δ 137.7, 133.2, 128.7, 128.5, 120.6, 41.1, 30.6, 25.7, 23.0.

HRMS (EI) calculated for [C₁₁H₁₀ClN]⁺ requires m/z 191.0496, found m/z 191.0501.

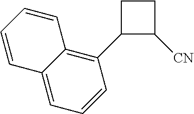 [SMILES: N#CC1CCC1c1cccc2ccccc12]

2-(Naphthalen-1-yl)cyclobutane-1-carbonitrile (23). The title compound was synthesized according to the General Procedure A using 1-vinylnaphthalene (30.8 mg, 0.2 mmol) and acrylonitrile (66 μL, 1.0 mmol). The crude mixture was purified by flash column chromatography using hexanes/ethyl acetate (10:1 v/v) as the eluent to give 28.1 mg (0.136 mmol, 68% yield, 1.6:1 d.r.) of the title compound as a colorless oil.

¹H NMR (CDCl₃, 400 MHz): δ 8.01 (d, J=8.8 Hz, 0.62H), 7.91-7.85 (m, 1H), 7.85-7.74 (m, 1.38H), 7.59-7.41 (m, 3.38H), 7.35 (d, J=7.2 Hz, 0.62H), 4.67-4.48 (m, 1H), 3.84-3.70 (m, 0.38H), 3.38-3.27 (m, 0.62H), 3.11-2.93 (m, 0.38H), 2.66-2.26 (m, 3.62H).

¹³C NMR (CDCl₃, 100 MHz): δ 136.8, 134.5, 133.8, 133.7, 131.02, 130.96, 129.1, 128.8, 128.1, 127.8, 126.4, 126.3, 126.0, 125.7, 125.34, 125.26, 124.4, 123.5, 122.6, 122.5, 121.4, 120.5, 42.5, 39.3, 31.9, 27.6, 27.2, 24.2, 23.4, 23.3.

HRMS (EI) calculated for [C₁₅H₁₃N]⁺ requires m/z 207.1043, found m/z 207.1046.

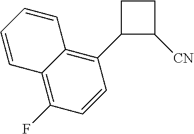 [SMILES: N#CC1CCC1c1ccc(F)c2ccccc12]

2-(4-Fluoronaphthalen-1-yl)cyclobutane-1-carbonitrile (24). The title compound was synthesized according to the General Procedure A using 1-fluoro-4-vinylnaphthalene (34.5 mg, 0.2 mmol) and acrylonitrile (66 μL, 1.0 mmol). The crude mixture was purified by flash column chromatography using hexanes/ethyl acetate (12:1 v/v) as the eluent to give 33.7 mg (0.150 mmol, 75% yield, 1.5:1 d.r.) of the title compound as a colorless oil.

Major: ¹H NMR (CDCl₃, 400 MHz): δ 8.20-8.11 (m, 1H), 8.06-7.96 (m, 1H), 7.69-7.54 (m, 2H), 7.30-7.24 (m, 1H), 7.18-7.06 (m, 1H), 4.57-4.39 (m, 1H), 3.35-3.16 (m, 1H), 2.67-2.27 (m, 4H).

¹⁹F NMR (376 MHz, CDCl₃): δ −123.9.

¹³C NMR (CDCl₃, 100 MHz): δ 158.2 (d, J=252.0 Hz), 132.7 (d, J=4.4 Hz), 132.2 (d, J=4.4 Hz), 127.4, 126.3 (d, J=1.9 Hz), 124.0 (d, J=16.3 Hz), 123.6 (d, J=2.8 Hz), 122.4 (d, J=8.7 Hz), 121.32 (d, J=4.8 Hz), 121.31, 108.6 (d, J=20.0 Hz), 42.1, 27.9, 27.1, 23.4.

Minor: ¹H NMR (CDCl₃, 400 MHz): δ 8.21-8.13 (m, 1H), 7.83-7.75 (m, 1H), 7.63-7.53 (m, 2H), 7.45-7.37 (m, 1H), 7.22-7.12 (m, 1H), 4.56-4.38 (m, 1H), 3.85-3.62 (m, 1H), 3.10-2.89 (m, 1H), 2.68-2.53 (m, 1H), 2.51-2.39 (m, 1H), 2.37-2.22 (m, 1H).

¹⁹F NMR (376 MHz, CDCl₃): δ −123.59.

¹³C NMR (CDCl₃, 100 MHz): δ 158.4 (d, J=252.3 Hz), 132.3 (d, J=4.4 Hz), 130.4 (d, J=4.3 Hz), 127.3, 126.1 (d, J=1.9 Hz), 124.4 (d, J=8.7 Hz), 124.0 (d, J=16.4 Hz), 122.7 (d, J=2.9 Hz), 121.7 (d, J=5.8 Hz), 120.4, 108.8 (d, J=20.1 Hz), 39.0, 31.9, 24.3, 23.3.

HRMS (EI) calculated for [C₁₅H₁₂FN]⁺ requires m/z 225.0948, found m/z 225.0958.

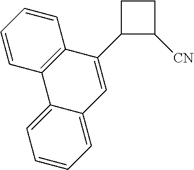 [SMILES: N#CC1CCC1c1cc2ccccc2c2ccccc12]

2-(Phenanthren-9-yl)cyclobutane-1-carbonitrile (25). The title compound was synthesized according to the General Procedure A using 9-vinylphenanthrene (40.9 mg, 0.2 mmol) and acrylonitrile (66 μL, 1.0 mmol). The crude mixture was purified by flash column chromatography using hexanes/ethyl acetate (10:1 v/v) as the eluent to give 38.6 mg (0.150 mmol, 75% yield, 1.4:1 d.r.) of the title compound as a colorless oil.

¹H NMR (CDCl₃, 400 MHz): δ 8.78-8.70 (m, 1H), 8.65 (d, J=8.1 Hz, 1H), 8.07-8.00 (m, 0.56H), 7.95-7.85 (m, 1H), 7.83-7.77 (m, 0.44H), 7.72-7.55 (m, 5H), 4.59-4.43 (m, 1H), 3.86-3.76 (m, 0.44H), 3.45-3.36 (m, 0.56H), 3.24-2.99 (m, 0.56H), 2.72-2.30 (m, 3.44H).

¹³C NMR (CDCl₃, 100 MHz): δ 135.2, 132.8, 131.4, 131.2, 130.8, 130.7, 130.13, 130.12, 130.0, 129.8, 128.8, 128.5, 126.93, 126.92, 126.81, 126.79, 126.76, 126.7, 126.5, 125.7, 124.3, 123.6, 123.5, 123.34, 123.26, 122.5, 121.5, 120.4, 42.7, 39.7, 31.9, 27.3, 27.2, 24.0, 23.44, 23.36.

HRMS (EI) calculated for [C₁₉H₁₅N]⁺ requires m/z 257.1199, found m/z 257.1207.

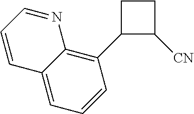 [SMILES: N#CC1CCC1c1cccc2cccnc12]

2-(Quinolin-8-yl)cyclobutane-1-carbonitrile (26). The title compound was synthesized according to the General Procedure A using 8-vinylquinoline (31.0 mg, 0.2 mmol) and acrylonitrile (66 μL, 1.0 mmol). The crude mixture was purified by flash column chromatography using hexanes/ethyl acetate (6:1 v/v) as the eluent to give 26.2 mg (0.126 mmol, 63% yield, 1.7:1 d.r.) of the title compound as a colorless oil.

¹H NMR (CDCl₃, 400 MHz): δ 8.95 (dd, J=4.2, 1.8 Hz, 0.62H), 8.88 (dd, J=4.2, 1.8 Hz, 0.38H), 8.19-8.12 (m, 1H), 7.79-7.71 (m, 1H), 7.70-7.65 (m, 0.38H), 7.61-7.48 (m, 1.62H), 7.45-7.39 (m, 1H), 4.98-4.76 (m, 1H), 3.98-3.85 (m, 0.38H), 3.54-3.40 (m, 0.62H), 2.97-2.86 (m, 0.38H), 2.68-2.21 (m, 3.62H).

¹³C NMR (CDCl₃, 100 MHz): δ 149.4, 146.4, 146.2, 139.5, 137.7, 136.4, 136.1, 128.3, 128.2, 127.4, 127.3, 127.1, 126.21, 126.19, 126.0, 122.1, 121.6, 121.3, 121.2, 42.1, 38.3, 32.3, 28.5, 26.9, 24.2, 23.8, 23.4.

HRMS (EI) calculated for [C₁₄H₁₂N₂₁ requires m/z 208.0995, found m/z 208.1004.

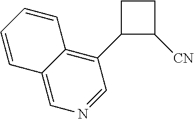 [SMILES: N#CC1CCC1c1cncc2ccccc12]

2-(Isoquinolin-4-yl)cyclobutane-1-carbonitrile (27). The title compound was synthesized according to the General Procedure A using 4-vinylisoquinoline (31.1 mg, 0.2 mmol) and acrylonitrile (66 μL, 1.0 mmol). The crude mixture was purified by flash column chromatography using hexanes/ethyl acetate (3:1 v/v) as the eluent to give 31.2 mg (0.150 mmol, 75% yield, 1.5:1 d.r.) of the title compound as a colorless oil.

¹H NMR (CDCl₃, 400 MHz): δ 9.24 (s, 0.38H), 9.20 (s, 0.62H), 8.53 (s, 0.38H), 8.44 (s, 0.62H), 8.11-7.98 (m, 1.62H), 7.84-7.73 (m, 1.38H), 7.72-7.60 (m, 1H), 4.59-4.39 (m, 1H), 3.90-3.70 (m, 0.38H), 3.39-3.26 (m, 0.62H), 3.16-3.02 (m, 0.38H), 2.71-2.34 (m, 3.62H).

¹³C NMR (CDCl₃, 100 MHz): δ 152.7, 152.4, 141.2, 139.7, 133.8, 133.7, 131.0, 130.9, 129.9, 128.8, 128.5, 128.2, 128.1, 127.5, 127.3, 122.7, 121.9, 121.1, 120.0, 40.5, 37.5, 31.5, 28.1, 26.2, 23.9, 23.72, 23.66.

HRMS (EI) calculated for [C₁₄H₁₂N₂]⁺ requires m/z 208.0995, found m/z 208.1001.

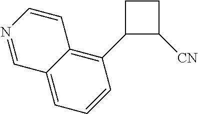 [SMILES: N#CC1CCC1c1cccc2cnccc12]

2-(Isoquinolin-5-yl)cyclobutane-1-carbonitrile (28). The title compound was synthesized according to the General Procedure A using 5-vinylisoquinoline (31.1 mg, 0.2 mmol) and acrylonitrile (66 μL, 1.0 mmol). The crude mixture was purified by flash column chromatography using hexanes/ethyl acetate (3:1 v/v) as the eluent to give 20.1 mg (0.096 mmol, 48% yield, 1.7:1 d.r.) of the title compound as a colorless oil.

¹H NMR (CDCl₃, 400 MHz): δ 9.31 (brs, 1H), 8.62 (brs, 1H), 8.02-7.87 (m, 1H), 7.85-7.79 (m, 0.60H), 7.76-7.72 (m, 0.40H), 7.71-7.56 (m, 2H), 4.66-4.26 (m, 1H), 3.85-3.68 (m, 0.40H), 3.31 (q, J=9.0 Hz, 0.60H), 3.14-2.93 (m, 0.40H), 2.67-2.33 (m, 3.60H).

¹³C NMR (CDCl₃, 100 MHz): δ 153.3, 153.1, 143.5, 143.4, 136.2, 134.0, 133.82, 133.78, 129.6, 128.7, 128.1, 127.6, 127.3, 127.0, 126.83, 126.79, 125.9, 123.7, 121.1, 120.1, 41.8, 38.5, 31.6, 27.8, 26.9, 24.2, 23.4, 23.3.

HRMS (EI) calculated for [C₁₄H₁₂N₂₁ requires m/z 208.0995, found m/z 208.1003.

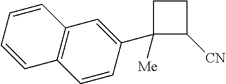 [SMILES: CC1(c2ccc3ccccc3c2)CCC1C#N]

2-Methyl-2-(naphthalen-2-yl)cyclobutane-1-carbonitrile (29). The title compound was synthesized according to the General Procedure A using 2-(prop-1-en-2-yl)naphthalene (68.0 mg, 0.4 mmol), acrylonitrile (132 μL, 2.0 mmol), 4-phenylmorpholine (39.2 mg, 0.24 mmol), UiO-69-phen(binap)Cu (35.6 mg, 6 μmol based on Cu, 1.5 mol %), and 1,2-dichloroethane (0.2 mL). The crude mixture was purified by flash column chromatography using hexanes/ethyl acetate (5:1 v/v) as the eluent to give 49.6 mg (0.224 mmol, 56% yield, 1.3:1 d.r.) of the title compound as a colorless oil.

Major: ¹H NMR (CDCl₃, 400 MHz): δ 7.86-7.78 (m, 3H), 7.56-7.43 (m, 3H), 7.32-7.26 (m, 1H), 3.43 (t, J=9.2 Hz, 1H), 2.63-2.44 (m, 2H), 2.41-2.30 (m, 1H), 2.25-2.16 (m, 1H), 1.73 (s, 3H).

¹³C NMR (CDCl₃, 100 MHz): δ 146.3, 133.2, 132.0, 128.6, 127.7, 127.6, 126.4, 125.8, 122.7, 122.3, 120.3, 45.8, 32.3, 32.0, 27.0, 21.3.

Minor: ¹H NMR (CDCl₃, 400 MHz): δ 7.87 (d, J=8.6 Hz, 1H), 7.85-7.80 (m, 2H), 7.65 (d, J=2.0 Hz, 1H), 7.51-7.43 (m, 2H), 7.35 (dd, J=8.6, 2.0 Hz, 1H), 3.28-3.19 (m, 1H), 3.07-2.96 (m, 1H), 2.68-2.56 (m, 1H), 2.32-2.16 (m, 2H), 1.65 (s, 3H).

¹³C NMR (CDCl₃, 100 MHz): δ 142.6, 133.3, 132.2, 128.4, 127.9, 127.7, 126.2, 125.8, 123.8, 123.7, 121.1, 46.6, 34.8, 31.2, 30.6, 21.1.

HRMS (EI) calculated for [C₁₆H₁₅N]⁺ requires m/z 221.1199, found m/z 221.1193.

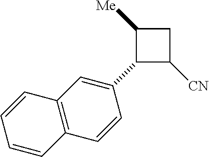 [SMILES: C[C@H]1CC(C#N)[C@@H]1c1ccc2ccccc2c1]

3-Methyl-2-(naphthalen-2-yl)cyclobutane-1-carbonitrile (30). The title compound was synthesized according to the General Procedure A using 2-(prop-1-en-1-yl)naphthalene (E:Z=4:1) (34.0 mg, 0.2 mmol) and acrylonitrile (66 μL, 1.0 mmol). The crude mixture was purified by flash column chromatography using hexanes to hexanes/ethyl acetate (10:1 v/v) as the eluent to give 39.2 mg (0.178 mmol, 89% yield, 1.1:1 d.r.) of the title compound as a colorless oil.

Major: ¹H NMR (CDCl₃, 400 MHz): δ 7.86-7.78 (m, 3H), 7.64 (s, 1H), 7.52-7.44 (m, 2H), 7.36 (dd, J=8.4, 1.8 Hz, 1H), 3.48 (t, J=9.4 Hz, 1H), 3.08-2.96 (m, 1H), 2.69-2.50 (m, 2H), 2.02 (q, J=10.0 Hz, 1H), 1.27 (d, J=6.4 Hz, 3H).

¹³C NMR (CDCl₃, 100 MHz): δ 137.7, 133.3, 132.6, 128.6, 127.69, 127.67, 126.4, 125.9, 124.8, 124.4, 121.4, 53.1, 36.1, 31.3, 25.9, 20.4.

Minor: ¹H NMR (CDCl₃, 400 MHz): δ 7.89-7.80 (m, 3H), 7.75-7.71 (m, 1H), 7.52-7.43 (m, 2H), 7.40 (dd, J=8.4, 1.8 Hz, 1H), 3.62-3.39 (m, 2H), 3.31-3.14 (m, 1H), 2.50-2.41 (m, 1H), 2.14-2.03 (m, 1H), 1.25 (d, J=6.4 Hz, 3H).

¹³C NMR (CDCl₃, 100 MHz): δ 135.9, 133.3, 132.8, 128.4, 127.9, 127.7, 126.2, 126.0, 125.9, 125.4, 121.2, 49.6, 34.8, 30.8, 28.3, 20.3.

HRMS (EI) calculated for [C₁₆H₁₅N]⁺ requires m/z 221.1199, found m/z 221.1187.

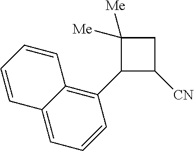 [SMILES: CC1(C)CC(C#N)C1c1cccc2ccccc12]

3,3-Dimethyl-2-(naphthalen-2-yl)cyclobutane-1-carbonitrile (31). The title compound was synthesized according to the General Procedure A using 2-(2-methylprop-1-en-1-yl)naphthalene (36.5 mg, 0.2 mmol), acrylonitrile (66 μL, 1.0 mmol), and UiO-69-phen(binap)Cu (5.9 mg, 1 μmol based on Cu, 0.5 mol %). The crude mixture was purified by flash column chromatography using hexanes to hexanes/ethyl acetate (10:1 v/v) as the eluent to give 45.1 mg (0.192 mmol, 96% yield, 1.2:1 d.r.) of the title compound as a colorless oil.

¹H NMR (CDCl₃, 400 MHz): δ 7.92-7.71 (m, 3.50H), 7.58-7.37 (m, 3H), 7.19 (d, J=8.4 Hz, 0.50H), 3.81-3.38 (m, 2H), 2.42-2.03 (m, 2H), 1.41-1.29 (m, 3H), 1.10 (s, 1.50H), 0.76 (s, 1.50H).

¹³C NMR (CDCl₃, 100 MHz): δ 135.0, 134.9, 133.24, 133.19, 132.43, 132.38, 128.1, 127.9, 127.8, 127.7, 127.6, 127.5, 126.8, 126.3, 126.14, 126.09, 125.80, 125.79, 125.1, 124.9, 122.0, 121.8, 54.1, 51.3, 40.6, 39.7, 37.5, 36.9, 30.8, 30.4, 24.2, 22.9, 22.2, 20.5.

HRMS (EI) calculated for [C₁₇H₁₇N]⁺ requires m/z 235.1356, found m/z 235.1356.

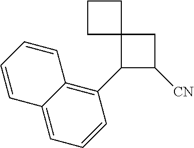 [SMILES: N#CC1CC2(CCC2)C1c1cccc2ccccc12]

1-(Naphthalen-2-yl)spiro[3.3]heptane-2-carbonitrile (32). The title compound was synthesized according to the General Procedure A using 2-(cyclobutylidenemethyl)naphthalene (38.9 mg, 0.2 mmol) and acrylonitrile (40 μL, 0.6 mmol). The crude mixture was purified by flash column chromatography using hexanes to hexanes/ethyl acetate (10:1 v/v) as the eluent to give 46.9 mg (0.190 mmol, 95% yield, 1.3:1 d.r.) of the title compound as a colorless oil.

¹H NMR (CDCl₃, 400 MHz): δ 7.90-7.80 (m, 3H), 7.81-7.73 (m, 0.44H), 7.59-7.54 (m, 0.56H), 7.53-7.43 (m, 2.44H), 7.29 (dd, J=8.5, 1.9 Hz, 0.56H), 3.79-3.64 (m, 1H), 3.59-3.45 (m, 0.44H), 3.34-3.21 (m, 0.56H), 2.75-2.63 (m, 0.44H), 2.61-2.54 (m, 0.44H), 2.51-2.36 (m, 1H), 2.23-1.63 (m, 5.56H), 1.57-1.49 (m, 0.56H).

¹³C NMR (CDCl₃, 100 MHz): δ 135.0, 134.9, 133.31, 133.28, 132.58, 132.57, 128.3, 128.1, 127.9, 127.7, 127.63, 127.59, 127.4, 126.3, 126.2, 126.1, 125.9, 125.8, 125.7, 125.3, 121.4, 121.0, 53.7, 52.2, 47.2, 46.9, 38.3, 37.1, 35.4, 33.3, 30.7, 28.8, 24.3, 21.7, 15.89, 15.86.

HRMS (EI) calculated for [C₁₈H₁₇N]⁺ requires m/z 247.1356, found m/z 247.1356.

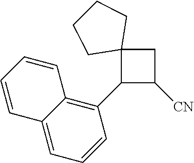 [SMILES: N#CC1CC2(CCCC2)C1c1cccc2ccccc12]

1-(Naphthalen-2-yl)spiro[3.4]octane-2-carbonitrile (33). The title compound was synthesized according to the General Procedure A using 2-(cyclopentylidenemethyl)naphthalene (41.6 mg, 0.2 mmol), acrylonitrile (40 μL, 0.6 mmol), and UiO-69-phen(binap)Cu (8.9 mg, 1.5 μmol based on Cu, 0.75 mol %). The crude mixture was purified by flash column chromatography using hexanes to hexanes/ethyl acetate (10:1 v/v) as the eluent to give 26.8 mg (0.102 mmol, 51% yield, 1.0:1 d.r.) of the title compound as a colorless oil.

¹H NMR (CDCl₃, 400 MHz): δ 7.89-7.77 (m, 3.50H), 7.58-7.42 (m, 3H), 7.26-7.21 (m, 0.50H), 3.96 (d, J=10.2 Hz, 0.50H), 3.76 (d, J=9.2 Hz, 0.50H), 3.67-3.55 (m, 0.50H), 3.42 (q, J=9.2 Hz, 0.50H), 2.54-2.20 (m, 2H), 1.95-1.76 (m, 2H), 1.65-1.26 (m, 6H).

¹³C NMR (CDCl₃, 100 MHz): δ 135.1, 135.0, 133.3, 133.2, 132.52, 132.50, 128.1, 127.90, 127.87, 127.7, 127.61, 127.56, 127.5, 126.5, 126.3, 126.1, 125.9, 125.8, 125.5, 125.4, 121.7, 121.4, 52.9, 51.4, 51.1, 51.0, 40.9, 39.9, 38.0, 37.6, 34.3, 32.6, 24.0, 23.7, 23.6, 23.5, 23.4, 21.4.

HRMS (EI) calculated for [C₁₉H₁₉N]⁺ requires m/z 261.1512, found m/z 261.1515.

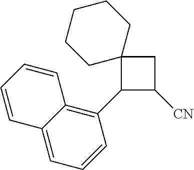 [SMILES: N#CC1CC2(CCCCC2)C1c1cccc2ccccc12]

1-(Naphthalen-2-yl)spiro[3.5]nonane-2-carbonitrile (34). The title compound was synthesized according to the General Procedure A using 2-(cyclohexylidenemethyl)naphthalene (44.5 mg, 0.2 mmol), acrylonitrile (40 μL, 0.6 mmol), and UiO-69-phen(binap)Cu (8.9 mg, 1.5 μmol based on Cu, 0.75 mol %). The crude mixture was purified by flash column chromatography using hexanes to hexanes/ethyl acetate (10:1 v/v) as the eluent to give 28.2 mg (0.102 mmol, 51% yield, 1.0:1 d.r.) of the title compound as a colorless oil.

¹H NMR (CDCl₃, 400 MHz): δ 7.92-7.73 (m, 3.50H), 7.58-7.41 (m, 3H), 7.26-7.19 (m, 0.50H), 3.70 (d, J=10.2 Hz, 0.50H), 3.67-3.55 (m, 1H), 3.47 (q, J=9.2 Hz, 0.50H), 2.44-2.08 (m, 2H), 1.88-0.76 (m, 10H).

¹³C NMR (CDCl₃, 100 MHz): δ 134.4, 134.24, 133.20, 133.1, 132.47, 132.45, 128.0, 127.9, 127.7, 127.6, 127.5, 127.0, 126.3, 126.1, 125.82, 125.77, 125.3, 122.1, 121.7, 54.5, 51.9, 44.1, 40.5, 39.5, 35.3, 33.9, 33.2, 30.9, 25.6, 25.5, 22.93, 22.91, 22.8, 21.90, 21.86, 20.5.

HRMS (EI) calculated for [C₂₀H₂₁N]⁺ requires m/z 275.1669, found m/z 275.1671.

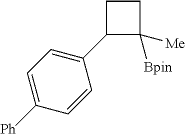

2-(2-([1,1′-Biphenyl]-4-yl)-1-methylcyclobutyl)-4,4,5,5-tetramethyl-1,3,2-dioxaborolane (35). The title compound was synthesized according to the General Procedure A using 4,4,5,5-tetramethyl-2-(prop-1-en-2-yl)-1,3,2-dioxaborolane (168 mg, 1.0 mmol) and 4-vinyl-1,1′-biphenyl (36.1 mg, 0.2 mmol). The crude mixture was purified by flash column chromatography using hexanes/ethyl acetate (30:1 v/v) as the eluent to give 31.3 mg (0.090 mmol, 45% yield, 3.4:1 d.r.) of the title compound as a colorless oil.

Major: ¹H NMR (CDCl₃, 400 MHz): δ 7.59 (d, J=8.0 Hz, 2H), 7.53 (d, J=8.0 Hz, 2H), 7.46-7.37 (m, 2H), 7.34-7.28 (m, 1H), 7.26-7.19 (m, 2H), 3.87-3.75 (m, 1H), 2.69-2.57 (m, 1H), 2.36-2.19 (m, 2H), 1.55-1.46 (m, 1H), 1.30 (s, 12H), 0.89 (s, 3H).

¹³C NMR (CDCl₃, 100 MHz): δ 141.6, 141.2, 138.3, 128.6, 127.9, 127.0, 126.9, 126.6, 83.2, 42.8, 27.1, 24.8, 24.7, 22.7, 17.5.

Minor: ¹H NMR (CDCl₃, 400 MHz): δ 7.55 (d, J=7.4 Hz, 2H), 7.48 (d, J=8.4 Hz, 2H), 7.44-7.39 (m, 2H), 7.34-7.31 (m, 1H), 7.30-7.27 (m, 2H), 3.33-3.23 (m, 1H), 2.55-2.44 (m, 1H), 2.22-2.11 (m, 2H), 1.70-1.61 (m, 1H), 1.38 (s, 3H), 0.95 (s, 6H), 0.91 (s, 6H).

¹³C NMR (CDCl₃, 100 MHz): δ 143.6, 141.5, 138.6, 128.7, 127.2, 127.0, 126.9, 126.6, 82.8, 50.4, 29.0, 26.2, 24.7, 24.6, 22.0.

HRMS (EI) calculated for [C₂₃H₂₉BO₂]⁺ requires m/z 348.2255, found m/z 348.2250.

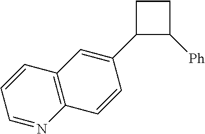 [SMILES: c1ccc(C2CCC2c2ccc3ncccc3c2)cc1]

6-(2-Phenylcyclobutyl)quinoline (36). The title compound was synthesized according to the General Procedure B using 6-vinylquinoline (311 mg, 2.0 mmol) and styrene (1.1 mL, 10 mmol). The crude mixture was purified by flash column chromatography using hexanes to hexanes/ethyl acetate (5:1 v/v) as the eluent to give 363 mg (1.40 mmol, 70% yield, 2.9:1 d.r.) of the title compound as a colorless oil.

¹H NMR (CDCl₃, 400 MHz): δ 9.37-8.96 (m, 1H), 8.59-8.37 (m, 1H), 7.90 (d, J=8.4 Hz, 0.75H), 7.70-7.57 (m, 1.75H), 7.53-7.46 (m, 1H), 7.44 (s, 0.25H), 7.36-7.29 (m, 1.50H), 7.27-7.20 (m, 2.25H), 7.13-6.91 (m, 1.50H), 4.28-4.08 (m, 0.50H), 3.84-3.63 (m, 1.50H), 2.65-2.49 (m, 1H), 2.48-2.35 (m, 1.50H), 2.32-2.18 (m, 1.50H).

¹³C NMR (CDCl₃, 100 MHz): δ 149.7, 149.5, 147.3, 146.9, 144.2, 142.9, 141.0, 140.2, 135.7, 135.6, 130.7, 129.3, 129.1, 128.4, 128.3, 128.2, 127.80, 127.78, 126.6, 126.2, 125.7, 125.4, 124.4, 121.1, 120.8, 47.9, 47.8, 45.3, 45.1, 26.0, 25.9, 24.3, 24.2.

HRMS (EI) calculated for [C₁₉H₁₇N]⁺ requires m/z 259.1356, found m/z 259.1360.

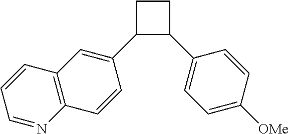 [SMILES: COc1ccc(C2CCC2c2ccc3ncccc3c2)cc1]

6-(2-(4-Methoxyphenyl)cyclobutyl)quinoline (37). The title compound was synthesized according to the General Procedure B using 6-vinylquinoline (312 mg, 2.0 mmol) and 1-methoxy-4-vinylbenzene (1.3 mL, 10 mmol). The crude mixture was purified by flash column chromatography using hexanes to hexanes/ethyl acetate (3:1 v/v) as the eluent to give 440 mg (1.52 mmol, 76% yield, 3.2:1 d.r.) of the title compound as a colorless oil.

¹H NMR (CDCl₃, 400 MHz): δ 8.86 (s, 1H), 8.15-7.75 (m, 2H), 7.66-7.28 (m, 2.52H), 7.25-7.11 (m, 2H), 6.92-6.81 (m, 2H), 6.57 (d, J=8.8 Hz, 0.48H), 4.25-4.02 (m, 0.48H), 3.83-3.55 (m, 4.52H), 2.62-2.47 (m, 0.96H), 2.45-2.31 (m, 1.52H), 2.28-2.13 (m, 1.52H).

¹³C NMR (CDCl₃, 100 MHz): δ 158.1, 157.5, 142.9, 140.3, 136.3, 135.71, 135.65, 133.2, 130.8, 129.30, 129.26, 129.2, 129.1, 128.8, 127.6, 125.4, 124.4, 113.82, 113.77, 113.7, 113.2, 55.2, 55.0, 48.2, 47.5, 45.1, 44.6, 26.3, 25.6, 24.6, 24.1.

HRMS (EI) calculated for [C₂₀H₁₉NO]⁺ requires m/z 289.1461, found m/z 289.1462.

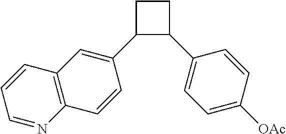 [SMILES: CC(=O)Oc1ccc(C2CCC2c2ccc3ncccc3c2)cc1]

4-(2-(Quinolin-6-yl)cyclobutyl)phenyl acetate (38). The title compound was synthesized according to the General Procedure B using 6-vinylquinoline (313 mg, 2.0 mmol) and 4-vinylphenyl acetate (1.6 g, 10 mmol). The crude mixture was purified by flash column chromatography using hexanes to hexanes/ethyl acetate (3:1 v/v) as the eluent to give 425 mg (1.34 mmol, 67% yield, 3.3:1 d.r.) of the title compound as a colorless oil.

¹H NMR (CDCl₃, 400 MHz): δ 8.86 (dd, J=4.2, 1.6 Hz, 0.77H), 8.79 (dd, J=4.2, 1.6 Hz, 0.23H), 8.10 (dd, J=8.4, 1.0 Hz, 0.77H), 8.06-8.02 (m, 0.77H), 8.01-7.98 (m, 0.23H), 7.80 (d, J=8.4 Hz, 0.23H), 7.65-7.57 (m, 1.54H), 7.41-7.34 (m, 1H), 7.33-7.29 (m, 0.23H), 7.28-7.21 (m, 1.77H), 7.05-6.99 (m, 1.54H), 6.95 (d, J=8.4 Hz, 0.46H), 6.76 (d, J=8.4 Hz, 0.46H), 4.30-3.99 (m, 0.46H), 3.81-3.59 (m, 1.54H), 2.63-2.46 (m, 0.92H), 2.46-2.34 (m, 1.54H), 2.32-2.16 (m, 4.54H).

¹³C NMR (CDCl₃, 100 MHz): δ 169.7, 169.4, 149.8, 149.6, 149.0, 148.6, 147.3, 146.9, 142.6, 141.8, 139.9, 138.7, 135.8, 135.7, 130.6, 129.4, 129.0, 128.7, 128.5, 128.2, 127.9, 127.5, 125.5, 124.4, 121.4, 121.2, 120.9, 120.8, 47.9, 47.3, 45.0, 44.7, 26.0, 25.9, 24.4, 24.2, 21.1, 21.0.

HRMS (EI) calculated for [C₂₁H₁₉NO₂]⁺ requires m/z 317.1410, found m/z 317.1410.

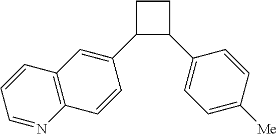 [SMILES: Cc1ccc(C2CCC2c2ccc3ncccc3c2)cc1]

6-(2-(p-Tolyl)cyclobutyl)quinoline (39). The title compound was synthesized according to the General Procedure B using 6-vinylquinoline (312 mg, 2.0 mmol) and 1-methyl-4-vinylbenzene (1.2 g, 10 mmol). The crude mixture was purified by flash column chromatography using hexanes to hexanes/ethyl acetate (3:1 v/v) as the eluent to give 355 mg (1.30 mmol, 65% yield, 3.1:1 d.r.) of the title compound as a colorless oil.

¹H NMR (CDCl₃, 400 MHz): δ 9.20 (s, 0.75H), 9.09 (s, 0.25H), 8.56-8.33 (m, 1H), 7.89 (d, J=8.4 Hz, 0.75H), 7.73-7.38 (m, 3H), 7.22-7.03 (m, 3.25H), 6.84 (s, 1H), 4.28-4.01 (m, 0.50H), 3.84-3.56 (m, 1.50H), 2.60-2.13 (m, 7H).

¹³C NMR (CDCl₃, 100 MHz): δ 149.7, 149.5, 147.2, 146.9, 143.0, 141.2, 140.4, 138.0, 135.8, 135.78, 135.72, 135.1, 130.8, 129.3, 129.08, 129.07, 128.5, 128.3, 128.20, 128.19, 127.7, 126.5, 125.4, 124.3, 121.1, 120.8, 47.9, 47.7, 45.0, 44.9, 26.1, 25.7, 24.4, 24.2, 21.0, 20.8.

HRMS (EI) calculated for [C₂₀H₁₉N]⁺ requires m/z 273.1512, found m/z 273.1514.

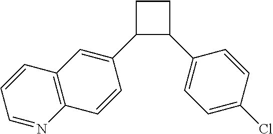 [SMILES: Clc1ccc(C2CCC2c2ccc3ncccc3c2)cc1]

6-(2-(4-Chlorophenyl)cyclobutyl)quinoline (40). The title compound was synthesized according to the General Procedure B using 6-vinylquinoline (311 mg, 2.0 mmol) and 1-chloro-4-vinylbenzene (1.4 g, 10 mmol). The crude mixture was purified by flash column chromatography using hexanes to hexanes/ethyl acetate (3:1 v/v) as the eluent to give 305 mg (1.04 mmol, 52% yield, 2.9:1 d.r.) of the title compound as a colorless oil.

¹H NMR (CDCl₃, 400 MHz): δ 9.21 (s, 0.75H), 9.10 (s, 0.25H), 8.57-8.39 (m, 1H), 7.90 (d, J=8.4 Hz, 1H), 7.70-7.41 (m, 3H), 7.30-7.26 (m, 1H), 7.21-7.05 (m, 2H), 7.02-6.96 (m, 0.50H), 6.91-6.79 (m, 0.50H), 4.25-4.06 (m, 0.50H), 3.79-3.58 (m, 1.50H), 2.68-2.49 (m, 1H), 2.47-2.35 (m, 1.50H), 2.32-2.14 (m, 1.50H).

¹³C NMR (CDCl₃, 100 MHz): δ 152.1, 152.0, 146.6, 143.1, 143.0, 142.9, 142.4, 136.0, 135.6, 132.0, 129.12, 129.07, 128.5, 128.4, 128.0, 127.9, 127.8, 127.6, 126.97, 126.95, 126.8, 126.5, 124.1, 123.11, 123.06, 120.3, 48.3, 47.3, 45.3, 44.6, 26.0, 25.5, 24.4, 23.8.

HRMS (EI) calculated for [C₁₉H₁₆ClN]⁺ requires m/z 293.0966, found m/z 293.0969.

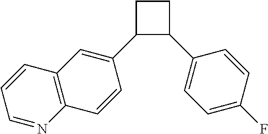 [SMILES: Fc1ccc(C2CCC2c2ccc3ncccc3c2)cc1]

6-(2-(4-Fluorophenyl)cyclobutyl)quinoline (41). The title compound was synthesized according to the General Procedure B using 6-vinylquinoline (313 mg, 2.0 mmol) and 1-fluoro-4-vinylbenzene (1.2 g, 10 mmol). The crude mixture was purified by flash column chromatography using hexanes to hexanes/ethyl acetate (4:1 v/v) as the eluent to give 372 mg (1.34 mmol, 67% yield, 2.8:1 d.r.) of the title compound as a colorless oil.

¹H NMR (CDCl₃, 400 MHz): δ 8.86 (dd, J=4.2, 1.8 Hz, 0.74H), 8.80 (dd, J=4.2, 1.8 Hz, 0.26H), 8.13-8.03 (m, 1.48H), 8.02-7.97 (m, 0.26H), 7.80 (d, J=8.8 Hz, 0.26H), 7.62-7.56 (m, 1.48H), 7.42-7.35 (m, 1H), 7.34-7.29 (m, 0.26H), 7.24-7.18 (m, 1.74H), 7.03-6.95 (m, 1.48H), 6.92-6.87 (m, 0.52H), 6.75-6.67 (m, 0.52H), 4.22-4.04 (m, 0.52H), 3.77-3.56 (m, 1.48H), 2.62-2.47 (m, 1.04H), 2.46-2.35 (m, 1.48H), 2.30-2.13 (m, 1.48H).

¹⁹F NMR (CDCl₃, 376 MHz): δ −116.9, −117.4.

¹³C NMR (CDCl₃, 100 MHz): δ 161.4 (d, J=244.1 Hz), 161.0 (d, J=243.8 Hz), 149.8, 149.6, 147.3, 146.9, 142.5, 139.9, 139.8 (d, J=3.2 Hz), 136.7 (d, J=3.2 Hz), 135.8, 135.7, 130.6, 129.4, 129.1 (d, J=7.8 Hz), 129.0, 128.5, 128.2, 128.0 (d, J=7.8 Hz), 127.9, 125.5, 124.4, 121.2, 120.9, 115.1 (d, J=21.2 Hz), 114.6 (d, J=21.1 Hz), 48.2, 47.4, 45.0, 44.5, 26.1, 25.7, 24.4, 23.9.

HRMS (EI) calculated for [C₁₉H₁₆FN]⁺ requires m/z 277.1261, found m/z 277.1262.

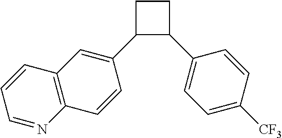 [SMILES: FC(F)(F)c1ccc(C2CCC2c2ccc3ncccc3c2)cc1]

6-(2-(4-(Trifluoromethyl)phenyl)cyclobutyl)quinoline (42). The title compound was synthesized according to the General Procedure B using 6-vinylquinoline (313 mg, 2.0 mmol) and 1-(trifluoromethyl)-4-vinylbenzene (1.7 g, mmol). The crude mixture was purified by flash column chromatography using hexanes to hexanes/ethyl acetate (5:1 v/v) as the eluent to give 563 mg (1.72 mmol, 86% yield, 3.3:1 d.r.) of the title compound as a colorless oil.

¹H NMR (CDCl₃, 400 MHz): δ 8.87 (dd, J=4.2, 1.6 Hz, 0.77H), 8.80 (dd, J=4.2, 1.6 Hz, 0.23H), 8.11 (dd, J=8.4, 1.6 Hz, 0.77H), 8.08-8.04 (m, 0.77H), 7.99 (dd, J=8.4, 1.6 Hz, 0.23H), 7.80 (d, J=8.7 Hz, 0.23H), 7.64-7.59 (m, 1.54H), 7.56 (d, J=8.2 Hz, 1.54H), 7.41-7.27 (m, 3.23H), 7.23 (dd, J=8.8, 2.0 Hz, 0.23H), 7.06 (d, J=8.4 Hz, 0.46H), 4.29-4.10 (m, 0.46H), 3.85-3.67 (m, 1.54H), 2.65-2.49 (m, 0.92H), 2.48-2.39 (m, 1.54H), 2.35-2.20 (m, 1.54H).

¹⁹F NMR (CDCl₃, 376 MHz): δ −62.3, −62.4.

¹³C NMR (CDCl₃, 100 MHz): δ 149.9, 149.7, 148.1, 147.2, 146.9, 145.3, 142.2, 139.5, 135.8, 135.7, 130.4, 129.5, 128.9, 128.67, 128.65, 128.5 (q, J=31.8 Hz), 128.2 (q, J=32.1 Hz), 128.0, 126.9, 125.6, 125.5, 125.3 (q, J=3.8 Hz), 124.7 (q, J=3.8 Hz), 124.5, 124.3 (q, J=270.2 Hz), 124.2 (q, J=270.2 Hz), 121.3, 121.0, 47.9, 47.6, 45.0, 25.9, 25.7, 24.24, 24.18.

HRMS (EI) calculated for [C₂₀H₁₆F₃N]⁺ requires m/z 327.1229, found m/z 327.1231.

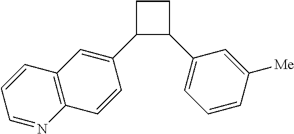 [SMILES: Cc1cccc(C2CCC2c2ccc3ncccc3c2)c1]

6-(2-(m-Tolyl)cyclobutyl)quinoline (43). The title compound was synthesized according to the General Procedure B using 6-vinylquinoline (313 mg, 2.0 mmol) and 1-methyl-3-vinylbenzene (1.2 g, 10 mmol). The crude mixture was purified by flash column chromatography using hexanes to hexanes/ethyl acetate (3:1 v/v) as the eluent to give 421 mg (1.54 mmol, 77% yield, 3.0:1 d.r.) of the title compound as a colorless oil.

¹H NMR (CDCl₃, 400 MHz): δ 8.85 (dd, J=4.4, 1.8 Hz, 0.75H), 8.78 (dd, J=4.4, 1.8 Hz, 0.25H), 8.12-8.07 (m, 0.75H), 8.06-7.98 (m, 1H), 7.79 (d, J=8.8 Hz, 0.25H), 7.64-7.56 (m, 1.50H), 7.41 (d, J=2.0 Hz, 0.25H), 7.36 (dd, J=8.4, 4.4 Hz, 0.75H), 7.30 (dd, J=8.4, 4.4 Hz, 0.25H), 7.24-7.17 (m, 1H), 7.09-7.01 (m, 2.25H), 6.93-6.88 (m, 0.25H), 6.79-6.71 (m, 0.75H), 4.24-4.02 (m, 0.50H), 3.85-3.57 (m, 1.50H), 2.61-2.47 (m, 1H), 2.44-2.35 (m, 1.50H), 2.33 (s, 2.25H), 2.27-2.18 (m, 1.50H), 2.10 (s, 0.75H).

¹³C NMR (CDCl₃, 100 MHz): δ 149.6, 149.3, 147.2, 146.8, 144.1, 143.0, 140.9, 140.4, 138.0, 137.1, 135.80, 135.75, 130.8, 129.3, 129.1, 128.7, 128.3, 128.21, 128.19, 127.8, 127.6, 127.3, 127.0, 126.5, 125.4, 124.8, 124.4, 123.7, 121.1, 120.8, 47.9, 47.7, 45.2, 45.1, 26.1, 25.9, 24.3, 24.2, 21.5, 21.3.

HRMS (EI) calculated for [C₂₀H₁₉N]⁺ requires m/z 273.1512, found m/z 273.1514.

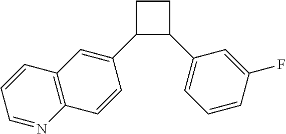 [SMILES: Fc1cccc(C2CCC2c2ccc3ncccc3c2)c1]

6-(2-(3-Fluorophenyl)cyclobutyl)quinoline (44). The title compound was synthesized according to the General Procedure B using 6-vinylquinoline (310 mg, 2.0 mmol) and 1-fluoro-3-vinylbenzene (1.2 g, 10 mmol). The crude mixture was purified by flash column chromatography using hexanes to hexanes/ethyl acetate (4:1 v/v) as the eluent to give 316 mg (1.14 mmol, 57% yield, 3.0:1 d.r.) of the title compound as a colorless oil.

¹H NMR (CDCl₃, 400 MHz): δ 8.86 (dd, J=4.4, 1.6 Hz, 0.75H), 8.79 (dd, J=4.4, 1.6 Hz, 0.25H), 8.10 (dd, J=8.4, 1.6 Hz, 0.75H), 8.07-8.02 (m, 0.75H), 8.00 (dd, J=8.4, 2.0 Hz, 0.25H), 7.80 (d, J=8.8 Hz, 0.25H), 7.65-7.58 (m, 1.50H), 7.41 (d, J=2.0 Hz, 0.25H), 7.37 (dd, J=8.4, 4.4 Hz, 0.75H), 7.31 (dd, J=8.4, 4.4 Hz, 0.25H), 7.28-7.26 (m, 0.25H), 7.26-7.21 (m, 0.75H), 7.03-6.87 (m, 2.50H), 6.74-6.59 (m, 0.75H), 4.27-4.03 (m, 0.50H), 3.80-3.60 (m, 1.50H), 2.66-2.49 (m, 1H), 2.46-2.35 (m, 1.50H), 2.31-2.14 (m, 1.50H).

¹⁹F NMR (CDCl₃, 376 MHz): δ −113.3, −114.1.

¹³C NMR (CDCl₃, 100 MHz): δ 163.0 (d, J=245.6 Hz), 162.6 (d, J=244.8 Hz), 149.8, 149.6, 147.3, 146.9, 146.8 (d, J=6.9 Hz), 143.9 (d, J=6.9 Hz), 142.4, 139.7, 135.8, 135.7, 130.5, 129.8 (d, J=8.3 Hz), 129.5, 129.1 (d, J=8.3 Hz), 128.9, 128.6, 128.2, 127.9, 125.4, 124.4, 123.6 (d, J=2.7 Hz), 122.3 (d, J=2.8 Hz), 121.2, 120.9, 114.5 (d, J=21.1 Hz), 113.4 (d, J=20.9 Hz), 113.1 (d, J=21.1 Hz), 112.6 (d, J=21.0 Hz), 47.8, 47.6 (d, J=1.7 Hz), 45.01, 44.96 (d, J=1.6 Hz), 25.9, 25.8, 24.13, 24.08.

HRMS (EI) calculated for [C₁₉H₁₆FN]⁺ requires m/z 277.1261, found m/z 277.1263.

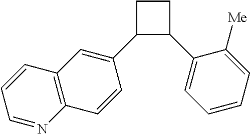 [SMILES: Cc1ccccc1C1CCC1c1ccc2ncccc2c1]

6-(2-(o-Tolyl)cyclobutyl)quinoline (45). The title compound was synthesized according to the General Procedure B using 6-vinylquinoline (311 mg, 2.0 mmol) and 1-methyl-2-vinylbenzene (1.2 g, 10 mmol). The crude mixture was purified by flash column chromatography using hexanes to hexanes/ethyl acetate (4:1 v/v) as the eluent to give 519 mg (1.90 mmol, 95% yield, 3.0:1 d.r.) of the title compound as a colorless oil.

¹H NMR (CDCl₃, 400 MHz): δ 8.84 (dd, J=4.4, 1.6 Hz, 0.75H), 8.76 (dd, J=4.4, 1.6 Hz, 0.25H), 8.16-7.98 (m, 1.50H), 7.93 (dd, J=8.4, 1.8 Hz, 0.25H), 7.75 (d, J=8.8 Hz, 0.25H), 7.68-7.56 (m, 1.50H), 7.45-7.33 (m, 1.75H), 7.30-7.26 (m, 0.50H), 7.24-7.17 (m, 1H), 7.14-7.11 (m, 1.50H), 7.05-6.98 (m, 0.25H), 6.96-6.85 (m, 0.50H), 4.33-4.14 (m, 0.50H), 4.05-3.67 (m, 1.50H), 2.75-2.56 (m, 1H), 2.50-2.41 (m, 1.50H), 2.29-2.18 (m, 3H), 2.12-2.01 (m, 1.50H).

¹³C NMR (CDCl₃, 100 MHz): δ 149.7, 149.5, 147.2, 146.9, 143.0, 141.9, 140.4, 138.6, 136.2, 135.9, 135.74, 135.71, 130.6, 130.2, 130.1, 129.8, 129.3, 129.0, 128.3, 128.2, 126.3, 126.1, 126.0, 125.9, 125.62, 125.60, 125.4, 124.3, 121.1, 120.8, 45.5, 45.2, 42.4, 26.7, 26.1, 25.2, 23.1, 19.74, 19.71.

HRMS (EI) calculated for [C₂₀H₁₉N]⁺ requires m/z 273.1512, found m/z 273.1514.

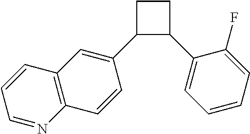 [SMILES: Fc1ccccc1C1CCC1c1ccc2ncccc2c1]

6-(2-(2-Fluorophenyl)cyclobutyl)quinoline (46). The title compound was synthesized according to the General Procedure B using 6-vinylquinoline (311 mg, 2.0 mmol) and 1-fluoro-2-vinylbenzene (1.2 g, 10 mmol). The crude mixture was purified by flash column chromatography using hexanes to hexanes/ethyl acetate (4:1 v/v) as the eluent to give 360 mg (1.30 mmol, 65% yield, 2.5:1 d.r.) of the title compound as a colorless oil.

¹H NMR (CDCl₃, 400 MHz): δ 8.85 (dd, J=4.4, 1.6 Hz, 0.71H), 8.77 (dd, J=4.4, 1.6 Hz, 0.29H), 8.09 (d, J=8.0 Hz, 0.71H), 8.04 (d, J=9.2 Hz, 0.71H), 7.99 (d, J=8.0 Hz, 0.29H), 7.78 (d, J=8.8 Hz, 0.29H), 7.65-7.58 (m, 1.42H), 7.45 (d, J=2.0 Hz, 0.29H), 7.41-7.28 (m, 2H), 7.21-7.17 (m, 0.71H), 7.15-7.09 (m, 0.71H), 7.07-7.05 (m, 0.29H), 7.01-6.94 (m, 0.71H), 6.94-6.88 (m, 0.29H), 6.88-6.78 (m, 0.29H), 6.76-6.66 (m, 0.29H), 4.44-4.17 (m, 0.58H), 4.08-3.77 (m, 1.42H), 2.71-2.34 (m, 2.58H), 2.32-2.10 (m, 1.42H).

¹⁹F NMR (CDCl₃, 376 MHz): δ −116.2, −117.1.

¹³C NMR (CDCl₃, 100 MHz): δ 160.8 (d, J=245.3 Hz), 160.6 (d, J=244.2 Hz), 149.8, 149.5, 147.3, 146.9, 142.6, 140.0, 135.8, 135.7, 130.8, 130.6, 130.4, 129.3, 129.0, 128.4, 128.21, 128.19, 128.1 (d, J=5.0 Hz), 128.0 (d, J=5.0 Hz), 127.8 (d, J=8.2 Hz), 127.5 (d, J=8.4 Hz), 125.3, 124.3, 124.1 (d, J=3.6 Hz), 123.4 (d, J=3.4 Hz), 121.1, 120.8, 115.3 (d, J=22.3 Hz), 114.5 (d, J=22.2 Hz), 46.3, 44.9, 41.2 (d, J=0.9 Hz), 38.5 (d, J=1.7 Hz), 26.6, 25.9 (d, J=1.9 Hz), 24.1, 22.7.

HRMS (EI) calculated for [C₁₉H₁₆FN]⁺ requires m/z 277.1261, found m/z 277.1260.

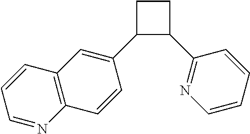 [SMILES: c1ccc(C2CCC2c2ccc3ncccc3c2)nc1]

6-(2-(Pyridin-2-yl)cyclobutyl)quinoline (47). The title compound was synthesized according to the General Procedure B using 6-vinylquinoline (312 mg, 2.0 mmol) and 2-vinylpyridine (1.1 g, 10 mmol). The crude mixture was purified by flash column chromatography using hexanes to hexanes/ethyl acetate (3:1 v/v) as the eluent to give 427 mg (1.64 mmol, 82% yield, 2.6:1 d.r.) of the title compound as a colorless oil.

¹H NMR (CDCl₃, 400 MHz): δ 8.85 (dd, J=4.4, 1.6 Hz, 0.72H), 8.77 (dd, J=4.4, 1.6 Hz, 0.28H), 8.67-8.60 (m, 0.72H), 8.40-8.33 (m, 0.28H), 8.09 (d, J=8.2 Hz, 0.72H), 8.02 (d, J=8.8 Hz, 0.72H), 8.00-7.96 (m, 0.28H), 7.75 (d, J=8.8 Hz, 0.28H), 7.66-7.55 (m, 2H), 7.43 (d, J=2.0 Hz, 0.28H), 7.36 (dd, J=8.4, 4.4 Hz, 0.72H), 7.33-7.20 (m, 1H), 7.18-7.10 (m, 1.44H), 6.90-6.78 (m, 0.56H), 4.36-4.18 (m, 0.56H), 4.14-4.01 (m, 0.72H), 3.84-3.73 (m, 0.72H), 2.79-2.24 (m, 4H).

¹³C NMR (CDCl₃, 100 MHz): δ 162.5, 160.7, 149.7, 149.6, 149.5, 148.7, 147.2, 146.8, 142.9, 140.1, 136.3, 135.74, 135.69, 135.5, 130.4, 129.3, 129.1, 128.3, 128.2, 127.8, 125.5, 124.3, 122.8, 121.8, 121.5, 121.1, 120.81, 120.78, 49.7, 47.1, 46.0, 44.7, 25.5, 24.9, 24.3, 22.6.

HRMS (EI) calculated for [C₁₈H₁₆N₂₁ requires m/z 260.1308, found m/z 260.1309.

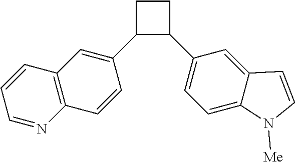 [SMILES: Cn1ccc2cc(C3CCC3c3ccc4ncccc4c3)ccc21]

6-(2-(1-Methyl-1H-indol-5-yl)cyclobutyl)quinoline (48). The title compound was synthesized according to the General Procedure B using 6-vinylquinoline (311 mg, 2.0 mmol) and 1-methyl-5-vinyl-1H-indole (1.6 g, 10 mmol). The crude mixture was purified by flash column chromatography using hexanes to hexanes/ethyl acetate (4:1 v/v) as the eluent to give 524 mg (1.68 mmol, 84% yield, 3.6:1 d.r.) of the title compound as a colorless oil.

¹H NMR (CDCl₃, 400 MHz): δ 8.83 (dd, J=4.4, 1.6 Hz, 0.78H), 8.73 (dd, J=4.4, 1.6 Hz, 0.22H), 8.07 (d, J=8.4 Hz, 0.78H), 8.01 (d, J=9.2 Hz, 0.78H), 7.96 (d, J=8.4 Hz, 0.22H), 7.71 (d, J=8.8 Hz, 0.22H), 7.65-7.59 (m, 1.56H), 7.53 (s, 0.78H), 7.46 (d, J=2.0 Hz, 0.22H), 7.37-7.31 (m, 1H), 7.28-7.23 (m, 1.22H), 7.14 (dd, J=8.4, 1.7 Hz, 0.78H), 7.02 (d, J=3.1 Hz, 0.78H), 6.91 (d, J=8.4 Hz, 0.22H), 6.88 (d, J=3.2 Hz, 0.22H), 6.73 (dd, J=8.4, 1.6 Hz, 0.22H), 6.43 (d, J=3.2 Hz, 0.78H), 6.28 (d, J=3.2 Hz, 0.22H), 4.31-4.17 (m, 0.44H), 3.86-3.70 (m, 3.90H), 3.61 (s, 0.66H), 2.64-2.52 (m, 0.88H), 2.49-2.36 (m, 1.56H), 2.34-2.18 (m, 1.56H).

¹³C NMR (CDCl₃, 100 MHz): δ 149.6, 149.2, 147.2, 146.8, 143.4, 140.9, 135.8, 135.7, 135.6, 135.2, 131.9, 131.0, 129.3, 129.2, 129.1, 128.52, 128.48, 128.21, 128.16, 128.1, 127.9, 125.3, 124.3, 122.2, 121.0, 120.8, 120.6, 119.4, 118.3, 109.1, 108.5, 100.6, 100.4, 48.5, 48.3, 45.4, 45.3, 32.8, 32.7, 26.8, 25.6, 25.0, 24.4.

HRMS (EI) calculated for [C₂₂H₂₀N₂]⁺ requires m/z 312.1621, found m/z 312.1623.

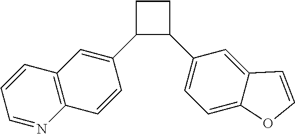 [SMILES: c1cnc2ccc(C3CCC3c3ccc4occc4c3)cc2c1]

6-(2-(Benzofuran-5-yl)cyclobutyl)quinoline (49). The title compound was synthesized according to the General Procedure B using 6-vinylquinoline (312 mg, 2.0 mmol) and 5-vinylbenzofuran (1.4 g, 10 mmol). The crude mixture was purified by flash column chromatography using hexanes to hexanes/ethyl acetate (5:1 v/v) as the eluent to give 479 mg (1.60 mmol, 80% yield, 3.8:1 d.r.) of the title compound as a colorless oil.

¹H NMR (CDCl₃, 400 MHz): δ 8.86 (s, 0.79H), 8.75 (s, 0.21H), 8.09 (d, J=8.4 Hz, 0.79H), 8.04 (d, J=9.2 Hz, 0.79H), 7.96 (dd, J=8.4, 1.6 Hz, 0.21H), 7.73 (d, J=8.8 Hz, 0.21H), 7.67-7.56 (m, 2.21H), 7.51-7.40 (m, 2H), 7.36 (dd, J=8.4, 4.4 Hz, 0.79H), 7.30-7.22 (m, 0.79H), 7.19 (dd, J=8.4, 1.6 Hz, 0.79H), 7.12 (d, J=8.4 Hz, 0.21H), 6.84 (dd, J=8.4, 1.6 Hz, 0.21H), 6.72 (d, J=2.2 Hz, 0.79H), 6.55 (d, J=2.2 Hz, 0.21H), 4.27-4.13 (m, 0.42H), 3.89-3.66 (m, 1.58H), 2.63-2.53 (m, 0.84H), 2.48-2.38 (m, 1.58H), 2.32-2.21 (m, 1.58H).

¹³C NMR (CDCl₃, 100 MHz): δ 153.8, 153.3, 149.7, 149.4, 147.3, 146.8, 145.2, 144.7, 142.9, 140.3, 138.8, 135.8, 135.65, 135.63, 130.7, 129.3, 129.1, 128.3, 128.2, 127.8, 127.5, 127.0, 125.4, 124.5, 124.4, 123.2, 121.1, 120.8, 119.8, 118.7, 111.1, 110.5, 106.5, 106.4, 48.4, 48.0, 45.23, 45.17, 26.5, 25.8, 24.8, 24.2.

HRMS (EI) calculated for [C₂₁H₁₇NO]⁺ requires m/z 299.1305, found m/z 299.1307.

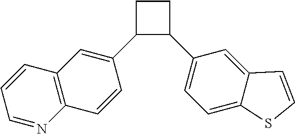 [SMILES: c1cnc2ccc(C3CCC3c3ccc4sccc4c3)cc2c1]

6-(2-(Benzo[b]thiophen-5-yl)cyclobutyl)quinoline (50). The title compound was synthesized according to the General Procedure B using 6-vinylquinoline (310 mg, 2.0 mmol) and 5-vinylbenzofuran (1.6 g, 2.0 mmol). The crude mixture was purified by flash column chromatography using hexanes to hexanes/ethyl acetate (4:1 v/v) as the eluent to give 536 mg (1.70 mmol, 85% yield, 3.0:1 d.r.) of the title compound as a colorless oil.

¹H NMR (CDCl₃, 400 MHz): δ 8.85 (dd, J=4.4, 1.6 Hz, 0.75H), 8.74 (dd, J=4.4, 1.6 Hz, 0.25H), 8.10-8.01 (m, 1.50H), 7.94 (dd, J=8.4, 1.6 Hz, 0.25H), 7.80 (d, J=8.4 Hz, 0.75H), 7.73 (d, J=8.8 Hz, 0.25H), 7.70 (s, 0.75H), 7.64-7.59 (m, 1.50H), 7.50-7.45 (m, 0.50H), 7.44-7.39 (m, 1H), 7.35 (dd, J=8.4, 4.4 Hz, 0.75H), 7.29-7.23 (m, 2.25H), 7.12 (d, J=5.4 Hz, 0.25H), 6.87 (dd, J=8.4, 1.6 Hz, 0.25H), 4.28-4.17 (m, 0.50H), 3.87-3.71 (m, 1.50H), 2.66-2.52 (m, 1H), 2.50-2.37 (m, 1.50H), 2.33-2.20 (m, 1.50H).

¹³C NMR (CDCl₃, 100 MHz): δ 149.7, 149.4, 147.2, 146.8, 142.8, 140.4, 140.2, 139.8, 139.4, 137.7, 137.4, 137.2, 135.8, 135.6, 130.7, 129.3, 129.1, 128.4, 128.2, 127.8, 126.7, 126.1, 125.4, 124.8, 124.4, 123.7, 123.6, 123.5, 122.4, 122.2, 121.7, 121.2, 121.1, 120.8, 48.1, 48.0, 45.15, 45.14, 26.3, 25.8, 24.6, 24.3.

HRMS (EI) calculated for [C₂₁H₁₇NS]⁺ requires m/z 315.1076, found m/z 315.1080.

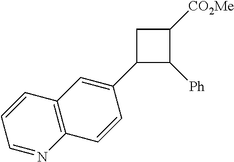 [SMILES: COC(=O)C1CC(c2ccc3ncccc3c2)C1c1ccccc1]

Methyl 2-phenyl-3-(quinolin-6-yl)cyclobutane-1-carboxylate (51). The title compound was synthesized according to the General Procedure B using 6-vinylquinoline (310 mg, 2.0 mmol) and methyl cinnamate (1.6 g, 10 mmol). The crude mixture was purified by flash column chromatography using hexanes/ethyl acetate (6:1-3:1 v/v) as the eluent to give 387 mg (1.22 mmol, 61% yield, 2.0:1 d.r.) of the title compound as a colorless oil.

¹H NMR (CDCl₃, 400 MHz): δ 9.40-8.97 (m, 1H), 8.62-8.38 (m, 1H), 7.94 (d, J=8.4 Hz, 0.67H), 7.69 (s, 0.67H), 7.68-7.61 (m, 1H), 7.60-7.52 (m, 1.33H), 7.36-7.24 (m, 3.33H), 7.10 (dd, J=8.4, 1.6 Hz, 0.33H), 7.06-6.97 (m, 1H), 6.94-6.87 (m, 0.67H), 4.40 (t, J=9.2 Hz, 0.33H), 4.24-4.16 (m, 0.33H), 3.96 (t, J=9.8 Hz, 0.67H), 3.81-3.66 (m, 4H), 3.36-3.26 (m, 0.67H), 2.98-2.85 (m, 0.33H), 2.80-2.68 (m, 1H), 2.55-2.50 (m, 0.67H).

¹³C NMR (CDCl₃, 100 MHz): δ 174.9, 174.4, 151.7, 151.4, 146.0, 143.9, 142.6, 142.0, 141.5, 138.5, 136.1, 135.8, 128.8, 128.6, 128.4, 128.1, 128.0, 127.5, 127.2, 127.1, 127.04, 127.00, 126.6, 126.4, 124.3, 123.7, 120.6, 52.03, 51.97, 50.7, 47.6, 43.7, 42.6, 41.5, 40.5, 29.2, 26.7.

HRMS (EI) calculated for [C₂₁H₁₉NO₂]⁺ requires m/z 317.1410, found m/z 317.1411.

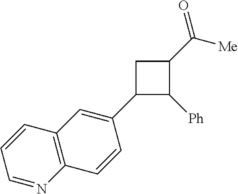 [SMILES: CC(=O)C1CC(c2ccc3ncccc3c2)C1c1ccccc1]

1-(2-Phenyl-3-(quinolin-6-yl)cyclobutyl)ethan-1-one (52). The title compound was synthesized according to the General Procedure B using 6-vinylquinoline (313 mg, 2.0 mmol) and (E)-4-phenylbut-3-en-2-one (1.5 g, 10 mmol). The crude mixture was purified by flash column chromatography using hexanes/ethyl acetate (6:1→3:1 v/v) as the eluent to give 361 mg (1.20 mmol, 60% yield, 2.0:1 d.r.) of the title compound as a colorless oil.

¹H NMR (CDCl₃, 400 MHz): δ 9.22 (s, 0.67H), 9.11 (s, 0.33H), 8.49 (d, J=6.0 Hz, 0.67H), 8.45 (d, J=6.0 Hz, 0.33H), 7.92 (d, J=8.4 Hz, 0.67H), 7.69-7.57 (m, 2H), 7.54 (s, 0.33H), 7.50-7.45 (m, 0.67H), 7.39-7.28 (m, 3.33H), 7.08-6.89 (m, 2H), 4.28-4.20 (m, 0.33H), 4.15-4.08 (m, 0.33H), 3.83-3.71 (m, 1.67H), 3.46-3.37 (m, 0.67H), 2.89-2.82 (m, 0.33H), 2.73-2.66 (m, 1H), 2.50-2.42 (m, 0.67H), 2.15 (s, 0.99H), 2.11 (s, 2.01H).

¹³C NMR (CDCl₃, 100 MHz): δ 208.3, 208.0, 151.4, 151.0, 146.4, 144.3, 142.0, 141.5, 141.4, 138.6, 136.2, 135.9, 129.1, 128.81, 128.77, 128.12, 128.09, 127.7, 127.2, 126.9, 126.6, 124.2, 123.5, 120.7, 50.6, 49.8, 48.4, 47.6, 43.0, 41.8, 28.5, 28.3, 27.8, 24.9.

HRMS (EI) calculated for [C₂₁H₁₉NO]⁺ requires m/z 301.1461, found m/z 301.1458.

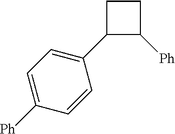 [SMILES: c1ccc(-c2ccc(C3CCC3c3ccccc3)cc2)cc1]

4-(2-Phenylcyclobutyl)-1,1′-biphenyl (53). The title compound was synthesized according to the General Procedure B using 4-vinyl-1,1′-biphenyl (360 mg, 2.0 mmol) and styrene (1.1 mL, 10 mmol). The crude mixture was purified by flash column chromatography using hexanes as the eluent to give 466 mg (1.64 mmol, 82% yield, 3.0:1 d.r.) of the title compound as a colorless oil.

¹H NMR (CDCl₃, 400 MHz): δ 7.61-7.47 (m, 3.50H), 7.45-7.17 (m, 9H), 7.10-6.94 (m, 1.50H), 4.11-3.98 (m, 0.50H), 3.68-3.55 (m, 1.50H), 2.54-2.42 (m, 1H), 2.41-2.27 (m, 1.50H), 2.24-2.09 (m, 1.50H).

¹³C NMR (CDCl₃, 100 MHz): δ 144.5, 143.7, 141.4, 141.1, 141.0, 140.7, 139.0, 138.2, 128.7, 128.6, 128.33, 128.32, 127.9, 127.7, 127.05, 127.02, 126.99, 126.9, 126.8, 126.6, 126.3, 126.1, 126.0, 125.6, 47.9, 47.6, 45.3, 45.0, 26.0, 25.9, 24.4, 24.2.

HRMS (EI) calculated for [C₂₂H₂₀]⁺ requires m/z 284.1560, found m/z 284.1564.

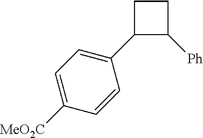 [SMILES: COC(=O)c1ccc(C2CCC2c2ccccc2)cc1]

Methyl 4-(2-phenylcyclobutyl)benzoate (54). The title compound was synthesized according to the General Procedure B using methyl 4-vinylbenzoate (324 mg, 2.0 mmol) and styrene (1.1 mL, 10 mmol). The crude mixture was purified by flash column chromatography using hexanes to hexanes/ethyl acetate (20:1 v/v) as the eluent to give 442 mg (1.66 mmol, 83% yield, 3.0:1 d.r.) of the title compound as a colorless oil.

¹H NMR (CDCl₃, 400 MHz): δ 7.96 (d, J=8.4 Hz, 1.50H), 7.75 (d, J=8.4 Hz, 0.50H), 7.33-7.26 (m, 3H), 7.24-7.17 (m, 2.25H), 7.10-7.04 (m, 0.50H), 7.03-6.97 (m, 0.75H), 6.94-6.89 (m, 0.50H), 4.11-3.98 (m, 0.50H), 3.89 (s, 2.25H), 3.83 (s, 0.75H), 3.67-3.53 (m, 1.50H), 2.53-2.43 (m, 1H), 2.39-2.29 (m, 1.50H), 2.23-2.11 (m, 1.50H).

¹³C NMR (CDCl₃, 100 MHz): δ 167.2, 167.1, 149.8, 147.2, 144.0, 140.9, 129.7, 129.0, 128.4, 128.0, 127.83, 127.78, 127.7, 127.3, 126.6, 126.5, 126.3, 125.7, 52.0, 51.8, 47.9, 47.8, 45.3, 45.2, 25.9, 25.6, 24.1, 24.0.

HRMS (EI) calculated for [C₁₈H₁₈O₂]⁺ requires m/z 266.1301, found m/z 266.1306.

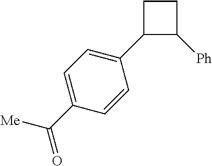 [SMILES: CC(=O)c1ccc(C2CCC2c2ccccc2)cc1]

1-(4-(2-Phenylcyclobutyl)phenyl)ethan-1-one (55). The title compound was synthesized according to the General Procedure B using 1-(4-vinylphenyl)ethan-1-one (292 mg, 2.0 mmol) and styrene (1.1 mL, 10 mmol). The crude mixture was purified by flash column chromatography using hexanes to hexanes/ethyl acetate (20:1 v/v) as the eluent to give 415 mg (1.66 mmol, 83% yield, 3.3:1 d.r.) of the title compound as a colorless oil.

¹H NMR (CDCl₃, 400 MHz): δ 7.89 (d, J=8.4 Hz, 1.54H), 7.68 (d, J=8.4 Hz, 0.46H), 7.34-7.27 (m, 3.08H), 7.24-7.18 (m, 2.23H), 7.12-7.06 (m, 0.46H), 7.04-6.99 (m, 0.77H), 6.96-6.89 (m, 0.46H), 4.16-4.02 (m, 0.46H), 3.70-3.52 (m, 1.54H), 2.58 (s, 2.31H), 2.54-2.43 (m, 1.61H), 2.40-2.30 (m, 1.54H), 2.24-2.12 (m, 1.54H).

¹³C NMR (CDCl₃, 100 MHz): δ 198.0, 197.8, 150.1, 147.6, 144.0, 140.9, 135.2, 134.6, 128.5, 128.4, 128.0, 127.84, 127.82, 127.7, 126.8, 126.5, 126.3, 125.8, 47.90, 47.86, 45.3, 45.2, 26.6, 26.5, 25.9, 25.6, 24.2, 24.1.

HRMS (EI) calculated for [C₁₈H₁₈O]⁺ requires m/z 250.1352, found m/z 250.1352.

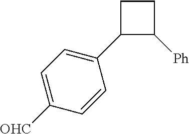 [SMILES: O=Cc1ccc(C2CCC2c2ccccc2)cc1]

4-(2-Phenylcyclobutyl)benzaldehyde (56). The title compound was synthesized according to the General Procedure B using 4-vinylbenzaldehyde (264 mg, 2.0 mmol) and styrene (1.1 mL, 10 mmol). The crude mixture was purified by flash column chromatography using hexanes to hexanes/ethyl acetate (20:1 v/v) as the eluent to give 402 mg (1.70 mmol, 85% yield, 3.6:1 d.r.) of the title compound as a colorless oil.

¹H NMR (CDCl₃, 400 MHz): δ 9.97 (s, 0.78H), 9.85 (s,0.22H), 7.81 (d, J=8.2 Hz, 1.56H), 7.59 (d, J=8.2 Hz, 0.44H), 7.40-7.19 (m, 5.44H), 7.12-6.90 (m, 1.56H), 4.16-3.98 (m, 0.44H), 3.75-3.51 (m, 1.56H), 2.58-2.45 (m, 0.88H), 2.43-2.28 (m, 1.56H), 2.27-2.11 (m, 1.56H).

¹³C NMR (CDCl₃, 100 MHz): δ 192.1, 192.0, 151.7, 149.2, 143.8, 140.8, 134.6, 134.0, 129.9, 129.2, 128.5, 128.4, 127.8, 127.7, 127.2, 126.6, 126.4, 125.9, 47.94, 47.93, 45.41, 45.38, 26.0, 25.6, 24.1, 24.0.

HRMS (EI) calculated for [C₁₇H₁₆O]⁺ requires m/z 236.1196, found m/z 236.1202.

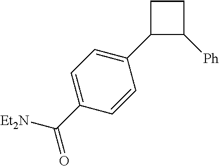

N,N-diethyl-4-(2-phenylcyclobutyl)benzamide (57). The title compound was synthesized according to the General Procedure B using N,N-diethyl-4-vinylbenzamide (407 mg, 2.0 mmol) and styrene (1.1 mL, 10 mmol). The crude mixture was purified by flash column chromatography using hexanes to hexanes/ethyl acetate (3:1 v/v) as the eluent to give 516 mg (1.68 mmol, 84% yield, 4.7:1 d.r.) of the title compound as a colorless oil.

¹H NMR (CDCl₃, 400 MHz): δ 7.33-7.17 (m, 7.36H), 7.11-6.89 (m, 1.64H), 4.17-3.94 (m, 0.36H), 3.73-2.96 (m, 5.64H), 2.56-2.42 (m, 0.72H), 2.41-2.25 (m, 1.64H), 2.22-2.06 (m, 1.64H), 1.28-1.05 (m, 6H).

¹³C NMR (CDCl₃, 100 MHz): δ 171.5, 171.4, 145.6, 144.2, 142.6, 141.1, 135.0, 134.3, 128.3, 127.9, 127.8, 127.6, 126.60, 126.58, 126.4, 126.2, 125.6, 125.5, 47.9, 47.7, 45.3, 45.1, 43.3, 39.2, 25.9, 25.8, 23.9, 23.7, 14.1, 13.0.

HRMS (EI) calculated for [C₂₁H₂₅NO]⁺ requires m/z 307.1931, found m/z 307.1935.

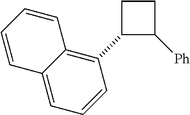 [SMILES: c1ccc(C2CC[C@H]2c2cccc3ccccc23)cc1]

Trans-1-(2-phenylcyclobutyl)naphthalene (58). The title compound was synthesized according to the General Procedure B using 1-vinylnaphthalene (308 mg, 2.0 mmol) and styrene (1.1 mL, 10 mmol). The crude mixture was purified by flash column chromatography using hexanes as the eluent to give 362 mg (1.40 mmol, 70% yield, >10:1 d.r.) of the title compound as a colorless oil.

¹H NMR (CDCl₃, 400 MHz): δ 7.96-7.89 (m, 1H), 7.87-7.80 (m, 1H), 7.71 (d, J=8.0 Hz, 1H), 7.53-7.39 (m, 4H), 7.35-7.26 (m, 4H), 7.23-7.15 (m, 1H), 4.27 (q, J=9.2 Hz, 1H), 3.99 (q, J=9.2 Hz, 1H), 2.71-2.59 (m, 1H), 2.50-2.39 (m, 1H), 2.33-2.21 (m, 1H), 2.16-2.04 (m, 1H).

¹³C NMR (CDCl₃, 100 MHz): δ 144.6, 140.4, 133.7, 131.6, 128.6, 128.3, 126.7, 126.6, 126.1, 125.57, 125.55, 125.5, 124.2, 122.8, 44.8, 44.7, 28.0, 26.6.

HRMS (EI) calculated for [C₂₀H₁₈]⁺ requires m/z 258.1403, found m/z 258.1409.

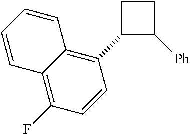 [SMILES: Fc1ccc([C@@H]2CCC2c2ccccc2)c2ccccc12]

Trans-1-fluoro-4-(2-phenylcyclobutyl)naphthalene (59). The title compound was synthesized according to the General Procedure B using 1-fluoro-4-vinylnaphthalene (343 mg, 2.0 mmol) and styrene (1.1 mL, 10 mmol). The crude mixture was purified by flash column chromatography using hexanes as the eluent to give 359 mg (1.30 mmol, 65% yield, >10:1 d.r.) of the title compound as a colorless oil.

¹H NMR (CDCl₃, 400 MHz): δ 8.13-8.10 (m, 1H), 7.89 (d, J=8.4 Hz, 1H), 7.56-7.44 (m, 2H), 7.42-7.35 (m, 1H), 7.32-7.27 (m, 4H), 7.22-7.17 (m, 1H), 7.13-7.05 (m, 1H), 4.28-4.12 (m, 1H), 3.99-3.85 (m, 1H), 2.69-2.55 (m, 1H), 2.50-2.38 (m, 1H), 2.33-2.19 (m, 1H), 2.14-1.99 (m, 1H).

¹⁹F NMR (CDCl₃, 376 MHz): δ −126.0.

¹³C NMR (CDCl₃, 100 MHz): δ 157.5 (d, J=250.1 Hz), 144.4, 136.2 (d, J=4.2 Hz), 132.7 (d, J=4.3 Hz), 128.4, 126.6, 126.5, 126.2, 125.8 (d, J=1.9 Hz), 124.2 (d, J=2.8 Hz), 123.9 (d, J=16.1 Hz), 122.4 (d, J=8.2 Hz), 121.0 (d, J=5.8 Hz), 108.7 (d, J=19.6 Hz), 45.0, 44.4, 27.9, 26.5.

HRMS (EI) calculated for [C₂₀H₁₇F]⁺ requires m/z 276.1309, found m/z 276.1315.

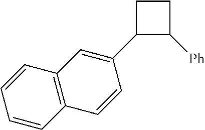 [SMILES: c1ccc(C2CCC2c2ccc3ccccc3c2)cc1]

2-(2-Phenylcyclobutyl)naphthalene (60). The title compound was synthesized according to the General Procedure B using 2-vinylnaphthalene (308 mg, 2.0 mmol) and styrene (1.1 mL, 10 mmol). The crude mixture was purified by flash column chromatography using hexanes as the eluent to give 418 mg (1.62 mmol, 81% yield, 6.7:1 d.r.) of the title compound as a colorless oil.

¹H NMR (CDCl₃, 400 MHz): δ 7.82-7.63 (m, 3.87H), 7.51-7.17 (m, 7.13H), 7.11-6.91 (m, 1H), 4.21-4.07 (m, 0.26H), 3.82-3.60 (m, 1.74H), 2.62-2.48 (m, 0.52H), 2.46-2.32 (m, 1.74H), 2.30-2.13 (m, 1.74H).

¹³C NMR (CDCl₃, 100 MHz): δ 144.5, 142.0, 141.5, 139.3, 133.5, 133.2, 132.2, 131.8, 128.3, 127.9, 127.7, 127.60, 127.58, 127.5, 127.4, 127.1, 127.0, 126.6, 126.1, 125.9, 125.7, 125.6, 125.5, 125.2, 124.9, 124.7, 48.2, 47.8, 45.3, 45.2, 26.0, 25.9, 24.4, 24.3.

HRMS (EI) calculated for [C₂₀H₁₈]⁺ requires m/z 258.1403, found m/z 258.1411.

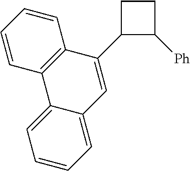 [SMILES: c1ccc(C2CCC2c2cc3ccccc3c3ccccc23)cc1]

Trans-9-(2-phenylcyclobutyl)phenanthrene (61). The title compound was synthesized according to the General Procedure B using 9-vinylphenanthrene (409 mg, 2.0 mmol) and styrene (1.1 mL, 10 mmol). The crude mixture was purified by flash column chromatography using hexanes as the eluent to give 493 mg (1.60 mmol, 80% yield, >10:1 d.r.) of the title compound as a colorless oil.

¹H NMR (CDCl₃, 400 MHz): δ 8.71 (d, J=7.6 Hz, 1H), 8.63 (d, J=7.6 Hz, 1H), 7.97 (dd, J=8.0, 1.2 Hz, 1H), 7.84 (dd, J=7.6, 1.6 Hz, 1H), 7.72 (s, 1H), 7.66-7.50 (m, 4H), 7.37-7.28 (m, 4H), 7.22-7.18 (m, 1H), 4.33-4.20 (m, 1H), 4.18-4.04 (m, 1H), 2.77-2.63 (m, 1H), 2.54-2.44 (m, 1H), 2.35-2.24 (m, 1H), 2.19-2.05 (m, 1H).

¹³C NMR (CDCl₃, 100 MHz): δ 144.8, 138.6, 131.8, 130.9, 130.6, 129.5, 128.8, 128.4, 126.7, 126.6, 126.4, 126.2, 126.12, 126.10, 124.8, 123.4, 123.1, 122.4, 45.0, 44.0, 28.2, 26.55.

HRMS (EI) calculated for [C₂₄H₂₀]⁺ requires m/z 308.1560, found m/z 308.1557.

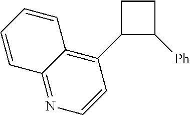 [SMILES: c1ccc(C2CCC2c2ccnc3ccccc23)cc1]

4-(2-Phenylcyclobutyl)quinoline (62). The title compound was synthesized according to the General Procedure B using 4-vinylquinoline (311 mg, 2.0 mmol) and styrene (1.1 mL, 10 mmol). The crude mixture was purified by flash column chromatography using hexanes to hexanes/ethyl acetate (6:1 v/v) as the eluent to give 410 mg (1.58 mmol, 79% yield, 4.5:1 d.r.) of the title compound as a colorless oil.

¹H NMR (CDCl₃, 400 MHz): δ 8.86 (d, J=4.4 Hz, 0.82H), 8.66 (d, J=4.4 Hz, 0.18H), 8.11 (d, J=8.4 Hz, 0.82H), 7.99-7.90 (m, 0.36H), 7.84 (dd, J=8.4, 1.6 Hz, 0.82H), 7.71-7.63 (m, 0.82H), 7.62-7.56 (m, 0.18H), 7.50-7.42 (m, 1H), 7.40 (dd, J=4.4, 1.2 Hz, 0.82H), 7.35-7.19 (m, 4.18H), 7.10 (d, J=4.4 Hz, 0.18H), 6.93-6.82 (m, 0.82H), 4.78-4.68 (m, 0.18H), 4.31-4.17 (m, 1H), 3.92 (q, J=9.4 Hz, 0.82H), 2.94-2.76 (m, 0.18H), 2.74-2.59 (m, 1H), 2.56-2.26 (m, 2H), 2.22-2.10 (m, 0.82H).

¹³C NMR (CDCl₃, 100 MHz): δ 150.4, 150.0, 149.8, 148.2, 147.7, 147.1, 143.7, 140.3, 130.0, 129.8, 129.0, 128.6, 128.5, 127.6, 127.4, 127.3, 127.0, 126.6, 126.5, 126.1, 126.0, 125.8, 124.00, 123.96, 118.9, 117.8, 46.5, 44.9, 44.1, 41.2, 27.3, 26.5, 24.4, 22.7.

HRMS (EI) calculated for [C₁₉H₁₇N]⁺ requires m/z 259.1356, found m/z 259.1360.

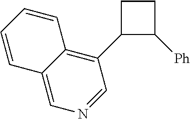 [SMILES: c1ccc(C2CCC2c2cncc3ccccc23)cc1]

Trans-4-(2-phenylcyclobutyl)isoquinoline (63). The title compound was synthesized according to the General Procedure B using 4-vinylisoquinoline (311 mg, 2.0 mmol) and styrene (1.1 mL, 10 mmol). The crude mixture was purified by flash column chromatography using hexanes to hexanes/ethyl acetate (5:1 v/v) as the eluent to give 322 mg (1.24 mmol, 62% yield, >10:1 d.r.) of the title compound as a colorless oil.

¹H NMR (CDCl₃, 400 MHz): δ 9.13 (s, 1H), 8.57 (s, 1H), 8.02-7.92 (m, 1H), 7.84-7.76 (m, 1H), 7.66-7.53 (m, 2H), 7.36-7.27 (m, 4H), 7.24-7.18 (m, 1H), 4.18 (q, J=9.2 Hz, 1H), 3.99 (q, J=9.2 Hz, 1H), 2.67-2.57 (m, 1H), 2.52-2.41 (m, 1H), 2.40-2.27 (m, 1H), 2.26-2.13 (m, 1H).

¹³C NMR (CDCl₃, 100 MHz): δ 151.3, 144.0, 140.2, 134.2, 133.1, 130.0, 128.4, 128.2, 126.8, 126.7, 126.4, 123.3, 45.3, 43.2, 27.1, 26.7.

HRMS (EI) calculated for [C₁₉H₁₇N]⁺ requires m/z 259.1356, found m/z 259.1354.

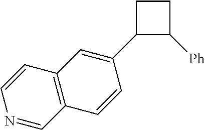 [SMILES: c1ccc(C2CCC2c2ccc3cnccc3c2)cc1]

6-(2-Phenylcyclobutyl)isoquinoline (64). The title compound was synthesized according to the General Procedure B using 6-vinylisoquinoline (311 mg, 2.0 mmol) and styrene (1.1 mL, 10 mmol). The crude mixture was purified by flash column chromatography using hexanes to hexanes/ethyl acetate (5:1 v/v) as the eluent to give 337 mg (1.30 mmol, 65% yield, 3.3:1 d.r.) of the title compound as a colorless oil.

¹H NMR (CDCl₃, 400 MHz): δ 8.85 (dd, J=4.4, 1.6 Hz, 0.75H), 8.77 (dd, J=4.4, 1.6 Hz, 0.25H), 8.08 (d, J=8.0 Hz, 0.75H), 8.04 (d, J=9.2 Hz, 0.75H), 7.98 (d, J=8.4 Hz, 0.25H), 7.77 (d, J=8.8 Hz, 0.25H), 7.63-7.59 (m, 1.25H), 7.40-7.19 (m, 5.75H), 7.07-7.00 (m, 0.50H), 6.98-6.93 (m, 0.50H), 4.25-4.06 (m, 0.50H), 3.85-3.60 (m, 1.50H), 2.63-2.48 (m, 1H), 2.46-2.34 (m, 1.50H), 2.30-2.16 (m, 1.50H).

¹³C NMR (CDCl₃, 100 MHz): δ 149.7, 149.5, 147.2, 146.8, 144.2, 142.9, 141.0, 140.2, 135.8, 135.7, 132.8, 130.7, 129.3, 129.1, 128.4, 128.3, 128.2, 127.80, 127.78, 126.6, 126.2, 125.7, 125.4, 124.4, 121.1, 120.8, 47.9, 47.8, 45.3, 45.1, 26.0, 25.9, 24.25, 24.16.

HRMS (EI) calculated for [C₁₉H₁₇N]⁺ requires m/z 259.1356, found m/z 259.1364.

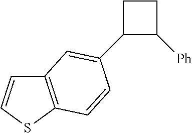 [SMILES: c1ccc(C2CCC2c2ccc3sccc3c2)cc1]

5-(2-Phenylcyclobutyl)benzo[b]thiophene (65). The title compound was synthesized according to the General Procedure B using 5-vinylbenzo[b]thiophene (320 mg, 2.0 mmol) and styrene (1.1 mL, 10 mmol). The crude mixture was purified by flash column chromatography using hexanes as the eluent to give 423 mg (1.60 mmol, 80% yield, 3.2:1 d.r.) of the title compound as a colorless oil.

¹H NMR (CDCl₃, 400 MHz): δ 7.78 (d, J=8.4 Hz, 0.76H), 7.68 (s, 0.76H), 7.54 (d, J=8.4 Hz, 0.24H), 7.45 (s, 0.24H), 7.42-7.37 (m, 0.76H), 7.32-7.16 (m, 5.76H), 7.07-7.02 (m, 0.48H), 7.00-6.94 (m, 0.76H), 6.86 (dd, J=8.4, 1.7 Hz, 0.24H), 4.20-3.94 (m, 0.48H), 3.78-3.52 (m, 1.52H), 2.58-2.44 (m, 0.96H), 2.43-2.29 (m, 1.52H), 2.26-2.10 (m, 1.52H).

¹³C NMR (CDCl₃, 100 MHz): δ 144.5, 141.5, 140.8, 139.8, 139.4, 137.8, 137.6, 137.0, 128.3, 127.9, 127.7, 126.6, 126.5, 126.1, 125.9, 125.5, 125.1, 123.7, 123.6, 122.3, 122.2, 121.5, 121.2, 48.1, 47.9, 45.3, 45.1, 26.3, 25.8, 24.7, 24.3.

HRMS (EI) calculated for [C₁₈H₁₆S]⁺ requires m/z 264.0967, found m/z 264.0971.

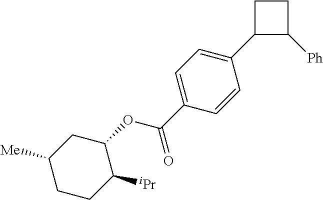 [SMILES: CC(C)[C@H]1CC[C@H](C)C[C@@H]1OC(=O)c1ccc(C2CCC2c2ccccc2)cc1]

2-Isopropyl-5-methylcyclohexyl 4-(2-phenylcyclobutyl)benzoate (66). The title compound was synthesized according to the General Procedure B using (1R,2S,5R)-2-isopropyl-5-methylcyclohexyl 4-vinylbenzoate (570 mg, 2.0 mmol) and styrene (1.1 mL, 10 mmol). The crude mixture was purified by flash column chromatography using hexanes to hexanes/ethyl acetate (v/v 20:1) as the eluent to give 500 mg (1.28 mmol, 64% yield, 3.2:1 d.r.) of the title compound as a colorless oil.

¹H NMR (CDCl₃, 400 MHz): δ 7.97 (d, J=8.4 Hz, 1.52H), 7.76 (d, J=8.4 Hz, 0.48H), 7.33-7.25 (m, 3.04H), 7.24-7.17 (m, 2.24H), 7.12-7.06 (m, 0.48H), 7.05-6.92 (m, 1.24H), 4.98-4.80 (m, 1H), 4.14-3.97 (m, 0.48H), 3.70-3.46 (m, 1.52H), 2.55-2.41 (m, 0.96H), 2.39-2.28 (m, 1.52H), 2.22-2.05 (m, 2.52H), 2.01-1.88 (m, 1H), 1.78-1.65 (m, 2H), 1.60-1.45 (m, 2H), 1.19-1.01 (m, 2H), 0.95-0.86 (m, 7H), 0.82-0.73 (m, 3H).

¹³C NMR (CDCl₃, 100 MHz): δ 166.2, 166.1, 149.6, 147.0, 144.0, 141.1, 129.7, 129.0, 128.7, 128.4, 128.0, 127.83, 127.80, 127.77, 126.54, 126.52, 126.3, 125.7, 74.6, 74.5, 47.90, 47.87, 47.3, 47.2, 45.21, 45.15, 41.0, 40.9, 34.3, 31.41, 31.38, 26.5, 26.4, 25.9, 25.7, 24.34, 24.26, 23.6, 22.0, 20.7, 16.5.

HRMS (EI) calculated for [C₂₇H₃₄O₂]⁺ requires m/z 390.2553, found m/z 390.2563.

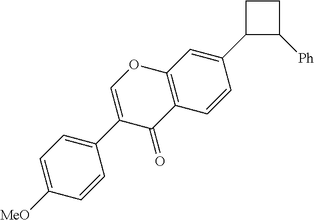 [SMILES: COc1ccc(-c2coc3cc(C4CCC4c4ccccc4)ccc3c2=O)cc1]

3-(4-Methoxyphenyl)-7-(2-phenylcyclobutyl)-4H-chromen-4-one (67). The title compound was synthesized according to the General Procedure B using 3-(4-methoxyphenyl)-7-vinyl-4H-chromen-4-one (555 mg, 2.0 mmol) and styrene (1.1 mL, 10 mmol). The crude mixture was purified by flash column chromatography using hexanes to hexanes/ethyl acetate (5:1 v/v) as the eluent to give 650 mg (1.70 mmol, 85% yield, 3.0:1 d.r.) of the title compound as a colorless oil.

¹H NMR (CDCl₃, 400 MHz): δ 8.21 (d, J=8.2 Hz, 0.75H), 7.98 (d, J=8.2 Hz, 0.25H), 7.95 (s, 0.75H), 7.87 (s, 0.25H), 7.53-7.44 (m, 2H), 7.38-7.21 (m, 5H), 7.13-6.89 (m, 4H), 4.19-4.06 (m, 0.50H), 3.87-3.81 (m, 3H), 3.75-3.56 (m, 1.50H), 2.60-2.45 (m, 1H), 2.44-2.31 (m, 1.50H), 2.29-2.14 (m, 1.50H).

¹³C NMR (CDCl₃, 100 MHz): δ 176.4, 176.3, 159.55, 159.47, 156.4, 156.0, 152.3, 152.2, 151.2, 148.8, 143.6, 140.6, 130.1, 130.0, 128.5, 128.0, 127.7, 126.6, 126.4, 126.3, 126.0, 125.6, 125.4, 124.9, 124.7, 124.3, 124.2, 124.1, 122.7, 122.1, 116.4, 115.4, 113.94, 113.90, 55.31, 55.28, 47.9, 47.8, 45.3, 45.2, 25.9, 25.6, 24.074, 24.068.

HRMS (EI) calculated for [C₂₆H₂₂O₃]⁺ requires m/z 382.1563, found m/z 382.1552.

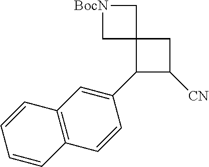 [SMILES: CC(C)(C)OC(=O)N1CC2(CC(C#N)C2c2ccc3ccccc3c2)C1]

Tert-butyl 6-cyano-5-(naphthalen-2-yl)-2-azaspiro[3.3]heptane-2-carboxylate (68). The title compound was synthesized according to the General Procedure C using tert-butyl 3-(naphthalen-2-ylmethylene)azetidine-1-carboxylate (59.2 mg, 0.2 mmol) and acrylonitrile (40 μL, 0.6 mmol). The crude mixture was purified by flash column chromatography using hexanes to hexanes/ethyl acetate (3:1 v/v) as the eluent to give 67.4 mg (0.194 mmol, 97% yield, 1.3:1 d.r.) of the title compound as a colorless oil.

¹H NMR (CDCl₃, 400 MHz): δ 7.93-7.80 (m, 3H), 7.74 (s, 0.44H), 7.57 (s, 0.56H), 7.55-7.47 (m, 2H), 7.39 (dd, J=8.4, 2.0 Hz, 0.44H), 7.26 (dd, J=8.4, 2.0 Hz, 0.56H), 4.18-3.84 (m, 4H), 3.66 (s, 1H), 3.62-3.51 (m, 0.44H), 3.37-3.22 (m, 0.56H), 2.83-2.56 (m, 2H), 1.48-1.24 (m, 9H).

¹³C NMR (CDCl₃, 100 MHz): δ 156.0, 155.9, 133.6, 133.31, 133.30, 133.28, 132.73, 132.70, 129.0, 128.8, 127.9, 127.72, 127.68, 127.6, 127.0, 126.6, 126.4, 126.3, 126.2, 125.5, 125.0, 124.3, 120.5, 119.9, 79.74, 79.73, 51.7, 50.3, 40.9, 40.8, 36.4, 34.9, 28.22, 28.19, 24.6, 21.8.

HRMS (EI) calculated for [C₂₂H₂₄N₂O₂]⁺ requires m/z 348.1832, found m/z 348.1830.

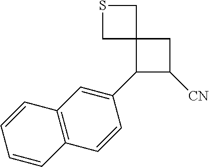 [SMILES: N#CC1CC2(CSC2)C1c1ccc2ccccc2c1]

5-(Naphthalen-2-yl)-2-thiaspiro[3.3]heptane-6-carbonitrile (69). The title compound was synthesized according to the General Procedure C using 3-(naphthalen-2-ylmethylene)thietane (42.6 mg, 0.2 mmol), acrylonitrile (40 μL, 0.6 mmol), and UiO-69-phen(binap)Cu (8.9 mg, 1.5 μmol based on Cu, 0.75 mol %). The crude mixture was purified by flash column chromatography using hexanes to hexanes/ethyl acetate (6:1 v/v) as the eluent to give 24.4 mg (0.092 mmol, 46% yield, 1.4:1 d.r.) of the title compound as a colorless oil.

¹H NMR (CDCl₃, 400 MHz): δ 7.94-7.82 (m, 3.41H), 7.61 (s, 0.59H), 7.56-7.48 (m, 2.41H), 7.39 (dd, J=8.4, 2.0 Hz, 0.59H), 3.80-3.73 (m, 1H), 3.63-3.52 (m, 1.41H), 3.34-3.16 (m, 2H), 3.08 (d, J=9.2 Hz, 0.59H), 3.03 (d, J=9.8 Hz, 0.41H), 2.90-2.73 (m, 2H), 2.60-2.52 (m, 0.59H).

¹³C NMR (CDCl₃, 100 MHz): δ 133.5, 133.34, 133.28, 133.1, 132.8, 132.7, 128.8, 128.6, 128.0, 127.8, 127.73, 127.67, 127.2, 126.6, 126.4, 126.3, 126.2, 125.6, 125.5, 124.9, 120.7, 120.3, 54.9, 53.1, 50.6, 50.3, 39.8, 38.7, 38.2, 36.9, 34.3, 32.7, 23.6, 21.0.

HRMS (EI) calculated for [C₁₇H₁₅NS]⁺ requires m/z 265.0920, found m/z 265.0918.

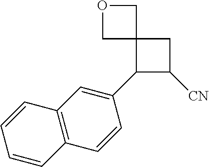 [SMILES: N#CC1CC2(COC2)C1c1ccc2ccccc2c1]

5-(Naphthalen-2-yl)-2-oxaspiro[3.3]heptane-6-carbonitrile (70). The title compound was synthesized according to the General Procedure C using 3-(naphthalen-2-ylmethylene)oxetane (39.3 mg, 0.2 mmol) and acrylonitrile (40 μL, 0.6 mmol). The crude mixture was purified by flash column chromatography using hexanes to hexanes/ethyl acetate (3:1 v/v) as the eluent to give 47.4 mg (0.190 mmol, 95% yield, 1.4:1 d.r.) of the title compound as a colorless oil.

Major: ¹H NMR (CDCl₃, 400 MHz): δ 7.91 (d, J=8.4 Hz, 1H), 7.88-7.81 (m, 2H), 7.64-7.60 (m, 1H), 7.56-7.47 (m, 2H), 7.36 (dd, J=8.4, 2.0 Hz, 1H), 4.83 (d, J=7.2 Hz, 1H), 4.75 (d, J=7.2 Hz, 1H), 4.46 (d, J=6.8 Hz, 1H), 4.42 (d, J=6.8 Hz, 1H), 4.02 (d, J=9.8 Hz, 1H), 3.25 (q, J=9.4 Hz, 1H), 2.79-2.60 (m, 2H).

¹³C NMR (CDCl₃, 100 MHz): δ 133.8, 133.3, 132.8, 129.0, 127.8, 127.7, 126.7, 126.3, 125.6, 124.4, 120.7, 81.0, 78.0, 51.6, 46.2, 34.2, 22.0.

Minor: ¹H NMR (CDCl₃, 400 MHz): δ 7.92 (d, J=8.4 Hz, 1H), 7.89-7.82 (m, 2H), 7.77 (s, 1H), 7.55-7.46 (m, 2H), 7.42 (dd, J=8.4, 2.0 Hz, 1H), 4.87 (d, J=6.8 Hz, 1H), 4.80 (d, J=6.8 Hz, 1H), 4.67 (q, J=7.2 Hz, 2H), 4.00 (d, J=8.8 Hz, 1H), 3.59-3.49 (m, 1H), 2.92-2.75 (m, 2H).

¹³C NMR (CDCl₃, 100 MHz): δ 133.4, 133.3, 132.8, 128.9, 127.9, 127.7, 127.0, 126.5, 126.3, 125.0, 120.0, 82.5, 78.9, 50.0, 46.2, 35.8, 24.6.

HRMS (EI) calculated for [C₁₇H₁₅NO]⁺ requires m/z 249.1148, found m/z 249.1152.

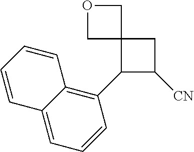 [SMILES: N#CC1CC2(COC2)C1c1cccc2ccccc12]

1-(Naphthalen-1-yl)spiro[3.3]heptane-2-carbonitrile (71). The title compound was synthesized according to the General Procedure C using 3-(naphthalen-1-ylmethylene)oxetane (39.4 mg, 0.2 mmol) and acrylonitrile (40 μL, 0.6 mmol). The crude mixture was purified by flash column chromatography using hexanes to hexanes/ethyl acetate (3:1 v/v) as the eluent to give 47.4 mg (0.190 mmol, 95% yield, >10:1 d.r.) of the title compound as a colorless oil.

¹H NMR (CDCl₃, 400 MHz): δ 8.11 (d, J=8.2 Hz, 1H), 7.92 (d, J=8.2 Hz, 1H), 7.85 (d, J=8.2 Hz, 1H), 7.64-7.45 (m, 3H), 7.19 (d, J=7.2 Hz, 1H), 4.98 (d, J=7.2 Hz, 1H), 4.76-4.65 (m, 2H), 4.37 (d, J=6.6 Hz, 1H), 4.19 (d, J=6.6 Hz, 1H), 3.42 (q, J=8.8 Hz, 1H), 2.88-2.68 (m, 2H).

¹³C NMR (CDCl₃, 100 MHz): δ 133.8, 133.3, 132.8, 129.0, 127.8, 127.7, 126.7, 126.3, 125.6, 124.4, 120.7, 81.0, 78.0, 51.6, 46.2, 34.2, 22.0.

HRMS (EI) calculated for [C₁₇H₁₅NO]⁺ requires m/z 249.1148, found m/z 249.1139.

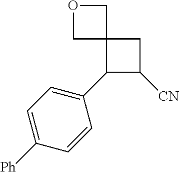 [SMILES: N#CC1CC2(COC2)C1c1ccc(-c2ccccc2)cc1]

1-([1,1′-Biphenyl]-4-yl)spiro[3.3]heptane-2-carbonitrile (72). The title compound was synthesized according to the General Procedure C using 3-([1,1′-biphenyl]-4-ylmethylene)oxetane (44.6 mg, 0.2 mmol) and acrylonitrile (40 μL, 0.6 mmol). The crude mixture was purified by flash column chromatography using hexanes to hexanes/ethyl acetate (2:1 v/v) as the eluent to give 46.3 mg (0.168 mmol, 84% yield, 1.3:1 d.r.) of the title compound as a colorless oil.

Major: ¹H NMR (CDCl₃, 400 MHz): δ 7.65 (d, J=8.2 Hz, 2H), 7.62-7.56 (m, 2H), 7.50-7.42 (m, 2H), 7.40-7.33 (m, 1H), 7.29 (d, J=8.2 Hz, 2H), 4.78 (d, J=7.2 Hz, 1H), 4.73 (d, J=7.2 Hz, 1H), 4.53-4.44 (m, 2H), 3.90 (d, J=9.8 Hz, 1H), 3.15 (q, J=9.4 Hz, 1H), 2.78-2.57 (m, 2H).

¹³C NMR (CDCl₃, 100 MHz): δ 140.9, 140.3, 135.2, 128.9, 127.8, 127.6, 127.2, 127.0, 120.6, 80.9, 78.0, 51.3, 46.2, 34.2, 22.1.

Minor: ¹H NMR (CDCl₃, 400 MHz): δ 7.65 (d, J=8.0 Hz, 2H), 7.61 (d, J=7.4 Hz, 2H), 7.49-7.42 (m, 2H), 7.42-7.32 (m, 3H), 4.83 (d, J=6.8 Hz, 1H), 4.77 (d, J=6.8 Hz, 1H), 4.73 (d, J=7.2 Hz, 1H), 4.65 (d, J=7.2 Hz, 1H), 3.87 (d, J=8.8 Hz, 1H), 3.49 (q, J=8.0 Hz, 1H), 2.80 (d, J=7.6 Hz, 2H).

¹³C NMR (CDCl₃, 100 MHz): δ 140.7, 140.4, 134.7, 128.8, 128.1, 127.7, 127.5, 127.1, 120.0, 82.4, 78.8, 49.5, 46.2, 35.7, 24.7.

HRMS (EI) calculated for [C₁₉H₁₇NO]⁺ requires m/z 275.1305, found m/z 275.1311.

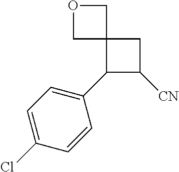 [SMILES: N#CC1CC2(COC2)C1c1ccc(Cl)cc1]

1-(4-Chlorophenyl)spiro[3.3]heptane-2-carbonitrile (73). The title compound was synthesized according to the General Procedure C using 3-(4-chlorobenzylidene)oxetane (36.2 mg, 0.2 mmol), acrylonitrile (40 μL, 0.6 mmol), and UiO-69-phen(binap)Cu (11.9 mg, 2.0 μmol based on Cu, 1.0 mol %). The crude mixture was purified by flash column chromatography using hexanes to hexanes/ethyl acetate (3:1 v/v) as the eluent to give 18.7 mg (0.080 mmol, 40% yield, 1.1:1 d.r.) of the title compound as a colorless oil.

¹H NMR (CDCl₃, 400 MHz): δ 7.46-7.36 (m, 2H), 7.25 (d, J=8.4 Hz, 1H), 7.17 (d, J=8.4 Hz, 1H), 4.77 (d, J=3.2 Hz, 1H), 4.72 (s, 1H), 4.63 (d, J=3.2 Hz, 1H), 4.47 (d, J=6.6 Hz, 0.50H), 4.40 (d, J=6.6 Hz, 0.50H), 3.83 (t, J=9.2 Hz, 1H), 3.57-3.41 (m, 0.50H), 3.11-3.02 (m, 0.50H), 2.87-2.55 (m, 2H).

¹³C NMR (CDCl₃, 100 MHz): δ 134.7, 134.3, 133.89, 133.88, 129.3, 129.2, 129.0, 128.1, 120.3, 119.7, 82.2, 80.8, 78.6, 77.7, 50.9, 49.2, 46.1, 35.5, 34.0, 24.6, 22.1.

HRMS (EI) calculated for [C₁₃H₁₂ClNO]⁺ requires m/z 233.0602, found m/z 233.0605.

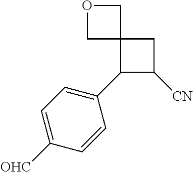 [SMILES: N#CC1CC2(COC2)C1c1ccc(C=O)cc1]

1-(4-Formylphenyl)spiro[3.3]heptane-2-carbonitrile (74). The title compound was synthesized according to the General Procedure C using 4-(oxetan-3-ylidenemethyl)benzaldehyde (34.8 mg, 0.2 mmol), acrylonitrile (40 μL, 0.6 mmol), and UiO-69-phen(binap)Cu (5.9 mg, 1.0 μmol based on Cu, 0.5 mol %). The crude mixture was purified by flash column chromatography using hexanes/ethyl acetate (10:1→1:1 v/v) as the eluent to give 27.3 mg (0.120 mmol, 60% yield, 1.3:1 d.r.) of the title compound as a colorless oil.

Major: ¹H NMR (CDCl₃, 400 MHz): δ 10.05 (s, 1H), 7.96 (d, J=8.0 Hz, 2H), 7.43 (d, J=8.0 Hz, 2H), 4.82-4.71 (m, 2H), 4.50 (d, J=6.8 Hz, 1H), 4.37 (d, J=6.8 Hz, 1H), 3.97 (d, J=10.0 Hz, 1H), 3.18 (q, J=9.4 Hz, 1H), 2.78-2.60 (m, 2H).

¹³C NMR (CDCl₃, 100 MHz): δ 191.4, 143.0, 135.9, 130.4, 127.4, 120.1, 80.8, 77.7, 51.3, 46.2, 34.0, 21.9.

Minor: ¹H NMR (CDCl₃, 400 MHz): δ 10.04 (s, 1H), 7.97 (d, J=8.0 Hz, 2H), 7.51 (d, J=8.0 Hz, 2H), 4.88-4.75 (m, 2H), 4.70-4.58 (m, 2H), 3.94 (d, J=8.8 Hz, 1H), 3.59-3.48 (m, 1H), 2.86-2.73 (m, 2H).

¹³C NMR (CDCl₃, 100 MHz): δ 191.6, 142.6, 135.8, 130.3, 128.3, 119.5, 82.1, 78.6, 49.6, 46.1, 35.5, 24.5.

HRMS (EI) calculated for [C₁₄H₁₃NO₂]⁺ requires m/z 227.0941, found m/z 227.0939.

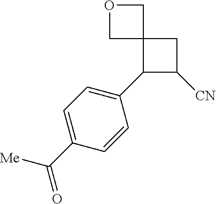 [SMILES: CC(=O)c1ccc(C2C(C#N)CC23COC3)cc1]

1-(4-Acetylphenyl)spiro[3.3]heptane-2-carbonitrile (75). The title compound was synthesized according to the General Procedure C using 1-(4-(oxetan-3-ylidenemethyl)phenyl)ethan-1-one (37.7 mg, 0.2 mmol) and acrylonitrile (40 μL, 0.6 mmol). The crude mixture was purified by flash column chromatography using hexanes to hexanes/ethyl acetate (2:1 v/v) as the eluent to give 44.9 mg (0.186 mmol, 93% yield, 1.3:1 d.r.) of the title compound as a colorless oil.

Major: ¹H NMR (CDCl₃, 400 MHz): δ 8.03 (d, J=8.0 Hz, 2H), 7.35 (d, J=8.0 Hz, 2H), 4.83-4.69 (m, 2H), 4.48 (d, J=6.8 Hz, 1H), 4.37 (d, J=6.8 Hz, 1H), 3.94 (d, J=9.8 Hz, 1H), 3.16 (q, J=9.4 Hz, 1H), 2.78-2.59 (m, 5H).

¹³C NMR (CDCl₃, 100 MHz): δ 197.3, 141.5, 136.6, 129.1, 126.9, 120.2, 80.8, 77.7, 51.2, 46.2, 34.1, 26.6, 21.9.

Minor: ¹H NMR (CDCl₃, 400 MHz): δ 8.03 (d, J=8.0 Hz, 2H), 7.42 (d, J=8.0 Hz, 2H), 4.81 (q, J=6.8 Hz, 2H), 4.64 (d, J=2.0 Hz, 2H), 3.91 (d, J=8.8 Hz, 1H), 3.57-3.44 (m, 1H), 2.86-2.74 (m, 2H), 2.63 (s, 3H).

¹³C NMR (CDCl₃, 100 MHz): δ 197.4, 141.1, 136.6, 129.0, 127.9, 119.6, 82.2, 78.6, 49.6, 46.1, 35.5, 26.6, 24.5.

HRMS (EI) calculated for [C₁₅H₁₅NO₂]⁺ requires m/z 241.1097, found m/z 241.1103.

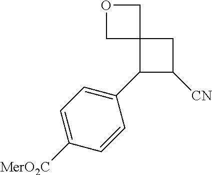

Methyl 4-(2-cyanospiro[3.3]heptan-1-yl)benzoate (76). The title compound was synthesized according to the General Procedure C using methyl 4-(oxetan-3-ylidenemethyl)benzoate (40.7 mg, 0.2 mmol) and acrylonitrile (40 μL, 0.6 mmol). The crude mixture was purified by flash column chromatography using hexanes to hexanes/ethyl acetate (2:1 v/v) as the eluent to give 39.7 mg (0.154 mmol, 77% yield, 1.3:1 d.r.) of the title compound as a colorless oil.

Major: ¹H NMR (CDCl₃, 400 MHz): δ 8.10 (d, J=8.4 Hz, 2H), 7.32 (d, J=8.4 Hz, 2H), 4.80-4.70 (m, 2H), 4.47 (d, J=6.8 Hz, 1H), 4.36 (d, J=6.8 Hz, 1H), 3.96-3.90 (m, 4H), 3.15 (q, J=9.4 Hz, 1H), 2.77-2.57 (m, 2H).

¹³C NMR (CDCl₃, 100 MHz): δ 166.5, 141.3, 130.4, 129.8, 126.7, 120.3, 80.8, 77.7, 52.2, 51.2, 46.2, 34.1, 21.9.

Minor: ¹H NMR (CDCl₃, 400 MHz): δ 8.11 (d, J=8.4 Hz, 2H), 7.40 (d, J=8.4 Hz, 2H), 4.85-4.76 (m, 2H), 4.63 (s, 2H), 3.93 (s, 3H), 3.90 (d, J=8.8 Hz, 1H), 3.56-3.44 (m, 1H), 2.84-2.73 (m, 2H).

¹³C NMR (CDCl₃, 100 MHz): δ 166.6, 140.9, 130.3, 129.8, 127.7, 119.6, 82.3, 78.6, 52.2, 49.7, 46.1, 35.6, 24.5.

HRMS (EI) calculated for [C₁₅H₁₅NO₃]⁺ requires m/z 257.1046, found m/z 257.1045.

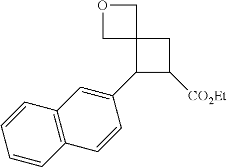 [SMILES: CCOC(=O)C1CC2(COC2)C1c1ccc2ccccc2c1]

Ethyl 1-(naphthalen-2-yl)spiro[3.3]heptane-2-carboxylate (77). The title compound was synthesized according to the General Procedure C using 3-(naphthalen-2-ylmethylene)oxetane (39.3 mg, 0.2 mmol) and ethyl acrylate (64 μL, 0.6 mmol). The crude mixture was purified by flash column chromatography using hexanes to hexanes/ethyl acetate (7:1 v/v) as the eluent to give 55.7 mg (0.188 mmol, 94% yield, 1.5:1 d.r.) of the title compound as a colorless oil.

¹H NMR (CDCl₃, 400 MHz): δ 7.90-7.76 (m, 3H), 7.68 (s, 0.40H), 7.62 (s, 0.60H), 7.52-7.40 (m, 2.40H), 7.28 (dd, J=8.4, 1.6 Hz, 0.60H), 4.90-4.79 (m, 1.60H), 4.74 (d, J=6.8 Hz, 0.40H), 4.66 (s, 1.20H), 4.46 (d, J=6.6 Hz, 0.40H), 4.41 (d, J=6.4 Hz, 0.40H), 4.20-4.11 (m, 0.80H), 4.04 (d, J=9.8 Hz, 0.60H), 3.92 (d, J=9.2 Hz, 0.40H), 3.82-3.73 (m, 1.20H), 3.56-3.45 (m, 0.60H), 3.30 (q, J=9.0 Hz, 0.40H), 2.90 (dd, J=12.6, 7.0 Hz, 0.60H), 2.61-2.48 (m, 1.40H), 1.24 (t, J=7.2 Hz, 1.20H), 0.79 (t, J=7.2 Hz, 1.80H).

¹³C NMR (CDCl₃, 100 MHz): δ 173.8, 173.0, 136.1, 135.4, 133.44, 133.35, 132.5, 132.3, 128.5, 128.1, 127.71, 127.66, 127.6, 127.5, 126.4, 126.2, 126.1, 125.8, 125.7, 125.6, 125.5, 125.3, 83.6, 81.8, 79.6, 78.8, 60.8, 60.3, 51.1, 50.1, 44.86, 44.84, 39.4, 37.6, 33.4, 33.0, 14.2, 13.7.

HRMS (EI) calculated for [C₁₉H₂₀O₃]⁺ requires m/z 296.1407, found m/z 296.1408.

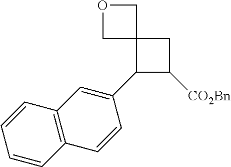 [SMILES: O=C(OCc1ccccc1)C1CC2(COC2)C1c1ccc2ccccc2c1]

Benzyl 1-(naphthalen-2-yl)spiro[3.3]heptane-2-carboxylate (78). The title compound was synthesized according to the General Procedure C using 3-(naphthalen-2-ylmethylene)oxetane (39.2 mg, 0.2 mmol) and benzyl acrylate (97.3 mg, 0.6 mmol). The crude mixture was purified by flash column chromatography using hexanes to hexanes/ethyl acetate (7:1 v/v) as the eluent to give 63.8 mg (0.178 mmol, 89% yield, 1.5:1 d.r.) of the title compound as a colorless oil.

¹H NMR (CDCl₃, 400 MHz): δ 7.90-7.70 (m, 3H), 7.67 (s, 0.40H), 7.59 (s, 0.60H), 7.52-7.38 (m, 2.40H), 7.34-7.26 (m, 2H), 7.22 (dd, J=8.4, 1.8 Hz, 0.60H), 7.15-7.07 (m, 0.60H), 7.03-6.94 (m, 1.20H), 6.80-6.72 (m, 1.20H), 5.13 (s, 0.80H), 4.87-4.77 (m, 1.60H), 4.76-4.66 (m, 1.60H), 4.60 (s, 1.20H), 4.45 (d, J=6.6 Hz, 0.40H), 4.41 (d, J=6.6 Hz, 0.40H), 4.03 (d, J=9.8 Hz, 0.60H), 3.93 (d, J=9.2 Hz, 0.40H), 3.62-3.50 (m, 0.60H), 3.36 (q, J=9.1 Hz, 0.40H), 2.94 (dd, J=12.7, 7.3 Hz, 0.60H), 2.63-2.47 (m, 1.40H).

¹³C NMR (CDCl₃, 100 MHz): δ 173.6, 172.8, 135.9, 135.7, 135.3, 135.1, 133.4, 132.5, 132.4, 128.5, 128.23, 128.19, 128.17, 128.1, 128.0, 127.9, 127.8, 127.7, 127.62, 127.56, 126.7, 126.2, 126.1, 125.8, 125.72, 125.68, 125.22, 125.20, 83.6, 81.7, 79.6, 78.7, 66.5, 66.4, 51.3, 50.2, 44.9, 44.7, 39.4, 37.6, 33.5, 32.9.

HRMS (EI) calculated for [C₂₄H₂₂O₃]⁺ requires m/z 358.1563, found m/z 358.1568.

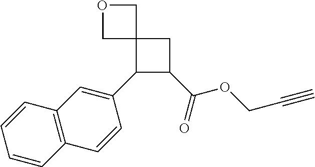 [SMILES: C#CCOC(=O)C1CC2(COC2)C1c1ccc2ccccc2c1]

Prop-2-yn-1-yl 1-(naphthalen-2-yl)spiro[3.3]heptane-2-carboxylate (79). The title compound was synthesized according to the General Procedure C using 3-(naphthalen-2-ylmethylene)oxetane (39.0 mg, 0.2 mmol) and prop-2-yn-1-yl acrylate (66.1 mg, 0.6 mmol). The crude mixture was purified by flash column chromatography using hexanes to hexanes/ethyl acetate (8:1 v/v) as the eluent to give 42.9 mg (0.140 mmol, 70% yield, 1.5:1 d.r.) of the title compound as a colorless oil.

¹H NMR (CDCl₃, 400 MHz): δ 7.91-7.76 (m, 3H), 7.70-7.65 (m, 0.40H), 7.63-7.59 (m, 0.60H), 7.53-7.38 (m, 2.40H), 7.28 (dd, J=8.4, 2.0 Hz, 0.60H), 4.89 (d, J=6.4 Hz, 0.60H), 4.87-4.80 (m, 1H), 4.78-4.64 (m, 2.40H), 4.47 (d, J=6.4 Hz, 0.40H), 4.41 (d, J=6.4 Hz, 0.40H), 4.40-4.31 (m, 0.60H), 4.31-4.22 (m, 0.60H), 4.14-4.02 (m, 0.60H), 3.95 (d, J=9.3 Hz, 0.40H), 3.64-3.52 (m, 0.60H), 3.37 (q, J=9.1 Hz, 0.40H), 2.99-2.89 (m, 0.60H), 2.63-2.53 (m, 1.40H), 2.47 (t, J=2.4 Hz, 0.40H), 2.03 (t, J=2.4 Hz, 0.60H).

¹³C NMR (CDCl₃, 100 MHz): δ 173.0, 172.2, 135.7, 135.0, 133.4, 132.5, 132.4, 128.6, 128.3, 127.9, 127.8, 127.7, 127.5, 126.5, 126.3, 126.1, 125.9, 125.8, 125.7, 125.4, 125.2, 83.5, 81.8, 79.6, 78.7, 77.4, 77.0, 75.1, 74.5, 52.3, 51.7, 51.2, 50.1, 44.9, 44.8, 39.3, 37.3, 33.4, 33.0.

HRMS (EI) calculated for [C₂₀H₁₈O₃]⁺ requires m/z 306.1250, found m/z 306.1245.

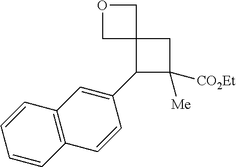 [SMILES: CCOC(=O)C1(C)CC2(COC2)C1c1ccc2ccccc2c1]

Ethyl 2-methyl-1-(naphthalen-2-yl)spiro[3.3]heptane-2-carboxylate (80). The title compound was synthesized according to the General Procedure C using 3-(naphthalen-2-ylmethylene)oxetane (39.2 mg, 0.2 mmol) and ethyl methacrylate (75 μL, 0.6 mmol). The crude mixture was purified by flash column chromatography using hexanes to hexanes/ethyl acetate (10:1 v/v) as the eluent to give 54.6 mg (0.176 mmol, 88% yield, 1.2:1 d.r.) of the title compound as a colorless oil.

Major: ¹H NMR (CDCl₃, 400 MHz): δ 7.88-7.80 (m, 3H), 7.75 (s, 1H), 7.52-7.44 (m, 2H), 7.36 (dd, J=8.4, 1.8 Hz, 1H), 5.00 (d, J=6.8 Hz, 1H), 4.93 (d, J=6.8 Hz, 1H), 4.87 (d, J=6.8 Hz, 1H), 4.70 (d, J=6.8 Hz, 1H), 4.23 (q, J=7.2 Hz, 2H), 4.07 (s, 1H), 2.86-2.77 (m, 1H), 2.29 (d, J=11.8 Hz, 1H), 1.32 (t, J=7.2 Hz, 3H), 1.07 (s, 3H).

¹³C NMR (CDCl₃, 100 MHz): δ 176.8, 134.9, 133.4, 132.2, 128.1, 127.8, 127.6, 127.0, 126.2, 126.1, 125.8, 83.5, 79.5, 61.0, 53.0, 44.0, 43.0, 42.2, 19.1, 14.2.

Minor: ¹H NMR (CDCl₃, 400 MHz): δ 7.82-7.75 (m, 3H), 7.60-7.56 (m, 1H), 7.49-7.40 (m, 2H), 7.26-7.22 (m, 1H), 4.92 (d, J=6.4 Hz, 1H), 4.85 (d, J=6.4 Hz, 1H), 4.71-4.63 (m, 2H), 3.78-3.66 (m, 2H), 3.62 (d, J=1.6 Hz, 1H), 3.13-2.99 (m, 1H), 2.25 (dd, J=12.5, 1.8 Hz, 1H), 1.56 (s, 3H), 0.71 (t, J=7.2 Hz, 3H).

¹³C NMR (CDCl₃, 100 MHz): δ 175.1, 135.9, 133.4, 132.2, 128.1, 127.7, 127.5, 126.1, 126.0, 125.6, 125.2, 84.0, 79.9, 60.4, 58.8, 45.7, 42.4, 41.1, 25.8, 13.6.

HRMS (EI) calculated for [C₂₀H₂₂O₃]⁺ requires m/z 310.1563, found m/z 310.1565.

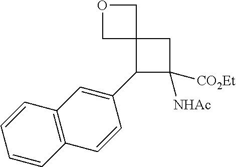 [SMILES: CCOC(=O)C1(NC(C)=O)CC2(COC2)C1c1ccc2ccccc2c1]

Methyl 2-acetamido-1-(naphthalen-2-yl)spiro[3.3]heptane-2-carboxylate (81). The title compound was synthesized according to the General Procedure C using 3-(naphthalen-2-ylmethylene)oxetane (39.2 mg, 0.2 mmol) and methyl 2-acetamidoacrylate (85.9 mg, 0.6 mmol). The crude mixture was purified by flash column chromatography using hexanes to hexanes/ethyl acetate (10:1 v/v) as the eluent to give 54.3 mg (0.160 mmol, 80% yield, 1.0:1 d.r.) of the title compound as a colorless oil.

¹H NMR (CDCl₃, 400 MHz): δ 7.91-7.77 (m, 3H), 7.65 (d, J=16.2 Hz, 1H), 7.56-7.44 (m, 2H), 7.36 (dd, J=8.6, 1.8 Hz, 0.50H), 7.25 (dd, J=8.6, 1.9 Hz, 0.50H), 6.80 (brs, 0.50H), 5.56 (brs, 0.50H), 4.97-4.87 (m, 2.50H), 4.77-4.71 (m, 1H), 4.67-4.59 (m, 1H), 4.15 (d, J=2.6 Hz, 0.50H), 3.81 (s, 1.50H), 3.48 (s, 1.50H), 3.30 (dd, J=13.2, 2.6 Hz, 0.50H), 3.16 (d, J=12.2 Hz, 0.50H), 2.92 (d, J=12.2 Hz, 0.50H), 2.72 (d, J=13.2 Hz, 0.50H), 2.05 (s, 1.50H), 1.68 (s, 1.50H).

¹³C NMR (CDCl₃, 100 MHz): δ 173.3, 173.1, 170.3, 170.0, 134.6, 133.4, 132.6, 132.2, 131.7, 128.8, 128.5, 128.4, 127.9, 127.7, 127.65, 127.60, 126.7, 126.5, 126.3, 125.85, 125.75, 124.6, 124.2, 83.9, 82.2, 79.3, 78.7, 60.5, 57.5, 55.5, 52.9, 52.6, 52.4, 42.6, 42.4, 42.1, 39.5, 24.0, 22.6.

HRMS (EI) calculated for [C₂₀H₂₁NO₄]⁺ requires m/z 339.1465, found m/z 339.1467.

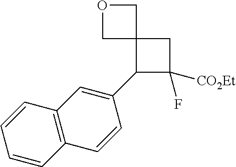 [SMILES: CCOC(=O)C1(F)CC2(COC2)C1c1ccc2ccccc2c1]

Methyl 2-fluoro-1-(naphthalen-2-yl)spiro[3.3]heptane-2-carboxylatee (82).

The title compound was synthesized according to the General Procedure C using 3-(naphthalen-2-ylmethylene)oxetane (39.2 mg, 0.2 mmol) and methyl 2-fluoroacrylate (56 μL, 0.6 mmol). The crude mixture was purified by flash column chromatography using hexanes to hexanes/ethyl acetate (10:1 v/v) as the eluent to give 45.0 mg (0.150 mmol, 75% yield, 1.6:1 d.r.) of the title compound as a colorless oil.

¹H NMR (CDCl₃, 400 MHz): δ 7.88-7.78 (m, 3.62H), 7.73 (s, 0.38H), 7.52-7.43 (m, 2.62H), 7.35 (d, J=8.4 Hz, 0.38H), 4.99 (d, J=7.4 Hz, 0.62H), 4.94-4.84 (m, 2H), 4.75 (d, J=7.4 Hz, 0.62H), 4.71-4.61 (m, 0.76H), 4.27-4.16 (m, 1H), 3.85 (s, 1.14H), 3.59 (s, 1.86H), 3.13-3.02 (m, 0.38H), 3.00-2.93 (m, 0.62H), 2.92-2.81 (m, 0.38H), 2.72-2.61 (m, 0.62H).

¹⁹F NMR (CDCl₃, 376 MHz): δ −142.1, −171.7.

¹³C NMR (CDCl₃, 100 MHz): δ 170.8 (d, J=27.1 Hz), 170.3 (d, J=27.5 Hz), 133.5, 133.3, 133.0, 132.6, 132.4, 131.1 (d, J=1.7 Hz), 128.7, 128.3, 128.1 (d, J=2.1 Hz), 127.9, 127.8, 127.64, 127.59, 126.34, 126.26, 126.13, 126.06 (d, J=2.2 Hz), 126.0, 124.8, 124.0, 92.7 (d, J=232.0 Hz), 92.5 (d, J=224.7 Hz), 82.1 (d, J=1.6 Hz), 80.9, 78.6 (d, J=2.8 Hz), 78.4, 56.1 (d, J=21.8 Hz), 55.6 (d, J=21.1 Hz), 52.9, 52.5, 42.71 (d, J=22.5 Hz), 42.70 (d, J=2.5 Hz), 41.7 (d, J=20.8 Hz), 39.4 (d, J=16.0 Hz).

HRMS (EI) calculated for [C₁₈H₁₇FO₃]⁺ requires m/z 300.1156, found m/z 300.1157.

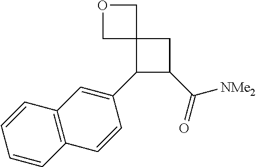 [SMILES: CN(C)C(=O)C1CC2(COC2)C1c1ccc2ccccc2c1]

N,N-Dimethyl-1-(naphthalen-2-yl)spiro[3.3]heptane-2-carboxamide (83).

The title compound was synthesized according to the General Procedure C using 3-(naphthalen-2-ylmethylene)oxetane (39.2 mg, 0.2 mmol) and N,N-dimethylacrylamide (59.5 mg, 0.6 mmol). The crude mixture was purified by flash column chromatography using hexanes to hexanes/ethyl acetate (1:1 v/v) as the eluent to give 44.9 mg (0.152 mmol, 76% yield, 1.4:1 d.r.) of the title compound as a colorless oil.

¹H NMR (CDCl₃, 400 MHz): δ 7.88-7.75 (m, 3H), 7.62 (s, 1H), 7.51-7.42 (m, 2H), 7.38 (dd, J=8.4, 2.0 Hz, 0.40H), 7.23 (dd, J=8.4, 2.0 Hz, 0.60H), 4.86-4.77 (m, 1.60H), 4.72 (d, J=6.8 Hz, 0.40H), 4.53 (d, J=7.0 Hz, 0.60H), 4.48 (q, J=6.6 Hz, 0.80H), 4.33 (d, J=7.0 Hz, 0.60H), 4.05-3.95 (m, 1H), 3.69-3.56 (m, 0.60H), 3.44 (q, J=8.6 Hz, 0.40H), 3.33-3.21 (m, 0.60H), 2.98-2.86 (m, 2.40H), 2.64-2.48 (m, 5H).

¹³C NMR (CDCl₃, 100 MHz): δ 172.9, 171.4, 136.7, 134.9, 133.5, 133.3, 132.49, 132.45, 131.2, 128.5, 128.1, 127.7, 127.64, 127.60, 127.5, 127.3, 126.2, 126.1, 126.0, 125.8, 125.7, 125.5, 84.1, 82.5, 79.6, 79.1, 53.0, 49.6, 44.4, 44.3, 38.1, 36.93, 36.86, 36.4, 35.6, 35.1, 33.7, 33.2.

HRMS (EI) calculated for [C₁₉H₂₁NO₂]⁺ requires m/z 295.1567, found m/z 295.1566.

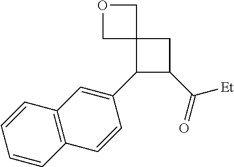 [SMILES: CCC(=O)C1CC2(COC2)C1c1ccc2ccccc2c1]

1-(1-(Naphthalen-2-yl)spiro[3.3]heptan-2-yl)propan-1-one (84). The title compound was synthesized according to the General Procedure C using 3-(naphthalen-2-ylmethylene)oxetane (39.2 mg, 0.2 mmol) and pent-1-en-3-one (50.5 mg, 0.6 mmol). The crude mixture was purified by flash column chromatography using hexanes to hexanes/ethyl acetate (5:1 v/v) as the eluent to give 53.8 mg (0.192 mmol, 96% yield, 1.3:1 d.r.) of the title compound as a colorless oil.

¹H NMR (CDCl₃, 400 MHz): δ 7.96-7.73 (m, 3H), 7.64 (s, 0.46H), 7.55 (s, 0.54H), 7.53-7.42 (m, 2H), 7.38 (dd, J=8.4, 1.8 Hz, 0.46H), 7.08 (dd, J=8.4, 1.8 Hz, 0.54H), 4.87 (d, J=6.2 Hz, 0.54H), 4.82 (d, J=6.2 Hz, 0.54H), 4.75 (d, J=6.8 Hz, 0.46H), 4.68 (d, J=6.8 Hz, 0.46H), 4.52 (d, J=7.0 Hz, 0.54H), 4.47 (d, J=6.5 Hz, 0.46H), 4.43-4.34 (m, 1H), 4.10 (d, J=10.1 Hz, 0.54H), 3.76 (d, J=9.1 Hz, 0.46H), 3.70-3.57 (m, 0.54H), 3.46 (q, J=8.9 Hz, 0.46H), 3.20-3.08 (m, 0.54H), 2.58-2.38 (m, 2.46H), 2.06-1.78 (m, 1H), 1.02 (t, J=7.3 Hz, 1.62H), 0.62 (t, J=7.3 Hz, 1.38H).

¹³C NMR (CDCl₃, 100 MHz): δ 210.5, 210.3, 136.1, 135.1, 133.5, 133.4, 132.6, 132.4, 128.7, 128.5, 127.7, 127.6, 127.3, 126.4, 126.3, 126.0, 125.92, 125.90, 125.52, 125.47, 84.4, 81.9, 79.4, 78.9, 53.2, 50.1, 45.8, 44.3, 43.9, 35.0, 34.5, 32.3, 32.2, 7.5, 7.2.

HRMS (EI) calculated for [C₁₉H₂₀O₂]⁺ requires m/z 280.1458, found m/z 280.1459.

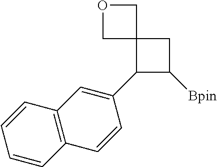

4,4,5,5-Tetramethyl-2-(1-(naphthalen-2-yl)spiro[3.3]heptan-2-yl)-1,3,2-dioxaborolane (85). The title compound was synthesized according to the General Procedure C using 3-(naphthalen-2-ylmethylene)oxetane (39.2 mg, 0.2 mmol) and 4,4,5,5-tetramethyl-2-vinyl-1,3,2-dioxaborolane (92.5 mg, 0.6 mmol). The crude mixture was purified by flash column chromatography using hexanes to hexanes/ethyl acetate (20:1 v/v) as the eluent to give 63.7 mg (0.182 mmol, 91% yield, 1.5:1 d.r.) of the title compound as a colorless oil.

Major: ¹H NMR (CDCl₃, 400 MHz): δ 7.84-7.75 (m, 3H), 7.70 (s, 1H), 7.49-7.39 (m, 3H), 4.90 (d, J=6.4 Hz, 1H), 4.83 (d, J=6.4 Hz, 1H), 4.63 (d, J=7.2 Hz, 1H), 4.59 (d, J=7.2 Hz, 1H), 3.89 (d, J=9.9 Hz, 1H), 2.57-2.48 (m, 1H), 2.47-2.37 (m, 1H), 2.35-2.24 (m, 1H), 0.94 (s, 6H), 0.93 (s, 6H).

¹³C NMR (CDCl₃, 100 MHz): δ 139.2, 133.4, 132.1, 127.9, 127.55, 127.54, 125.89, 125.85, 125.6, 125.2, 84.0, 83.2, 80.3, 49.2, 47.0, 32.3, 24.7, 24.6.

Minor: ¹H NMR (CDCl₃, 400 MHz): δ 7.90-7.78 (m, 3H), 7.64 (s, 1H), 7.51-7.40 (m, 3H), 4.82 (d, J=6.8 Hz, 1H), 4.70 (d, J=6.8 Hz, 1H), 4.43 (d, J=6.4 Hz, 1H), 4.39 (d, J=6.4 Hz, 1H), 3.72 (d, J=9.6 Hz, 1H), 2.43-2.24 (m, 2H), 2.03 (q, J=9.5 Hz, 1H), 1.24 (s, 12H).

¹³C NMR (CDCl₃, 100 MHz): δ 138.7, 133.6, 132.3, 128.1, 127.7, 127.6, 126.0, 125.8, 125.44, 125.41, 83.4, 82.6, 79.7, 48.5, 48.1, 31.5, 24.75, 24.68.

HRMS (EI) calculated for [C₂₂H₂₇BO₃]⁺ requires m/z 350.2048, found m/z 350.2052.

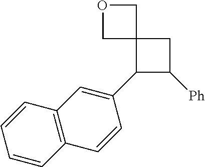 [SMILES: c1ccc(C2CC3(COC3)C2c2ccc3ccccc3c2)cc1]

5-(Naphthalen-2-yl)-6,6-diphenyl-2-oxaspiro[3.3]heptane (86). The title compound was synthesized according to the General Procedure C using 3-(naphthalen-2-ylmethylene)oxetane (39.2 mg, 0.2 mmol) and styrene (68 μL, 0.6 mmol). The crude mixture was purified by flash column chromatography using hexanes to hexanes/ethyl acetate (20:1 v/v) as the eluent to give 58.9 mg (0.196 mmol, 98% yield, 1.5:1 d.r.) of the title compound as a colorless oil.

¹H NMR (CDCl₃, 400 MHz): δ 7.92-7.78 (m, 1.20H), 7.72-7.64 (m, 1.60H), 7.56-7.44 (m, 1.80H), 7.42-7.34 (m, 1.80H), 7.30-7.17 (m, 2H), 7.09-7.01 (m, 1.20H), 7.00-6.94 (m, 0.60H), 6.93-6.85 (m, 1.80H), 5.04 (d, J=6.0 Hz, 0.60H), 4.97 (d, J=6.0 Hz, 0.60H), 4.85 (d, J=6.7 Hz, 0.40H), 4.74 (d, J=6.7 Hz, 0.40H), 4.62-4.50 (m, 1.40H), 4.44 (d, J=6.9 Hz, 0.60H), 4.21-4.15 (m, 0.60H), 4.10-3.98 (m, 0.60H), 3.82-3.67 (m, 0.80H), 3.07-2.98 (m, 0.60H), 2.92-2.83 (m, 0.60H), 2.80-2.70 (m, 0.40H), 2.45-2.31 (m, 0.40H).

¹³C NMR (CDCl₃, 100 MHz): δ 143.6, 140.2, 137.1, 135.7, 133.5, 133.2, 132.5, 131.9, 128.5, 128.4, 127.8, 127.72, 127.71, 127.66, 127.6, 127.5, 127.4, 127.2, 126.8, 126.4, 126.2, 125.9, 125.82, 125.78, 125.7, 125.6, 125.3, 85.3, 81.4, 79.4, 79.2, 55.2, 53.8, 44.9, 43.6, 38.9, 38.4, 37.2, 36.7.

HRMS (EI) calculated for [C₂₂H₂₀O]⁺ requires m/z 300.1509, found m/z 300.1508.

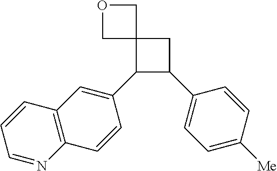 [SMILES: Cc1ccc(C2CC3(COC3)C2c2ccc3ncccc3c2)cc1]

5-(Naphthalen-2-yl)-6-(p-tolyl)-2-oxaspiro[3.3]heptane (87). The title compound was synthesized according to the General Procedure C using 3-(naphthalen-2-ylmethylene)oxetane (39.2 mg, 0.2 mmol) and 1-methyl-4-vinylbenzene (77 μL, 0.6 mmol). The crude mixture was purified by flash column chromatography using hexanes to hexanes/ethyl acetate (20:1 v/v) as the eluent to give 57.2 mg (0.182 mmol, 91% yield, 1.5:1 d.r.) of the title compound as a colorless oil.

¹H NMR (CDCl₃, 400 MHz): δ 7.91-7.76 (m, 1.20H), 7.72-7.63 (m, 1.60H), 7.54 (d, J=8.5 Hz, 0.60H), 7.51-7.33 (m, 3H), 7.16-7.05 (m, 1.60H), 6.90 (dd, J=8.5, 1.8 Hz, 0.60H), 6.85 (d, J=8.0 Hz, 1.20H), 6.78 (d, J=8.0 Hz, 1.20H), 5.02 (d, J=5.9 Hz, 0.60H), 4.96 (d, J=6.0 Hz, 0.60H), 4.84 (d, J=6.7 Hz, 0.40H), 4.73 (d, J=6.7 Hz, 0.40H), 4.61-4.50 (m, 1.40H), 4.44 (d, J=6.9 Hz, 0.60H), 4.14 (dd, J=9.3, 3.0 Hz, 0.60H), 3.99 (q, J=9.4 Hz, 0.60H), 3.77-3.62 (m, 0.80H), 3.03-2.95 (m, 0.60H), 2.89-2.80 (m, 0.60H), 2.78-2.69 (m, 0.40H), 2.37-2.28 (m, 1.60H), 2.14 (s, 1.80H).

¹³C NMR (CDCl₃, 100 MHz): δ 140.5, 137.2, 137.1, 135.9, 135.0, 133.5, 133.3, 132.5, 131.9, 129.1, 128.5, 128.4, 127.70, 127.68, 127.64, 127.60, 127.5, 127.4, 127.1, 126.9, 126.3, 126.1, 125.9, 125.8, 125.7, 125.6, 125.3, 85.3, 81.5, 79.4, 79.2, 55.1, 53.9, 44.9, 43.6, 38.7, 38.2, 37.4, 36.9, 21.0, 20.9.

HRMS (EI) calculated for [C₂₃H₂₂O]⁺ requires m/z 314.1665, found m/z 314.1665.

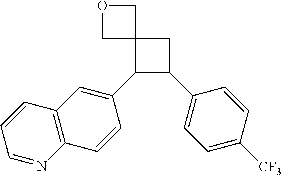 [SMILES: FC(F)(F)c1ccc(C2CC3(COC3)C2c2ccc3ncccc3c2)cc1]

5-(Naphthalen-2-yl)-6-(4-(trifluoromethyl)phenyl)-2-oxaspiro[3.3]heptane (88). The title compound was synthesized according to the General Procedure C using 3-(naphthalen-2-ylmethylene)oxetane (39.2 mg, 0.2 mmol) and 1-(trifluoromethyl)-4-vinylbenzene (103 mg, 0.6 mmol). The crude mixture was purified by flash column chromatography using hexanes to hexanes/ethyl acetate (8:1 v/v) as the eluent to give 60.4 mg (0.164 mmol, 82% yield, 1.5:1 d.r.) of the title compound as a colorless oil.

Major: ¹H NMR (CDCl₃, 400 MHz): δ 7.94-7.80 (m, 3H), 7.71-7.66 (m, 1H), 7.57-7.44 (m, 5H), 7.36-7.29 (m, 2H), 4.85 (d, J=6.8 Hz, 1H), 4.74 (d, J=6.8 Hz, 1H), 4.60 (d, J=6.2 Hz, 1H), 4.53 (d, J=6.2 Hz, 1H), 3.88-3.67 (m, 2H), 2.88-2.76 (m, 1H), 2.46-2.29 (m, 1H).

¹⁹F NMR (CDCl₃, 376 MHz): δ −62.38.

¹³C NMR (CDCl₃, 100 MHz): δ 147.6, 136.4, 133.5, 132.6, 130.9, 128.68 (q, J=32.3 Hz), 128.66, 127.7, 126.7, 126.4, 125.90, 125.86, 125.6, 125.4 (q, J=3.7 Hz), 124.2 (q, J=270.2 Hz), 81.2, 79.0, 53.8, 45.0, 38.3, 37.0.

Minor: ¹H NMR (CDCl₃, 400 MHz): δ 7.74-7.66 (m, 2H), 7.55 (d, J=8.6 Hz, 1H), 7.46-7.37 (m, 3H), 7.30 (d, J=8.0 Hz, 2H), 7.00 (d, J=8.0 Hz, 2H), 6.85 (dd, J=8.6, 1.8 Hz, 1H), 5.05 (d, J=6.0 Hz, 1H), 4.98 (d, J=6.0 Hz, 1H), 4.55 (d, J=7.0 Hz, 1H), 4.43 (d, J=7.0 Hz, 1H), 4.24-4.20 (m, 1H), 4.11-4.06 (m, 1H), 3.09-2.90 (m, 2H).

¹⁹F NMR (CDCl₃, 376 MHz): δ −62.36.

¹³C NMR (CDCl₃, 100 MHz): δ 144.6, 135.1, 133.3, 132.0, 127.9 (q, J=18.2 Hz), 127.9, 127.8, 127.54, 127.53, 127.4, 126.4, 126.1, 125.6, 124.8 (q, J=3.8 Hz), 124.1 (q, J=272.0 Hz), 85.1, 79.3, 55.2, 43.6, 38.8, 36.9.

HRMS (EI) calculated for [C₂₃H₁₉F₃O]⁺ requires m/z 368.1383, found m/z 368.1374.

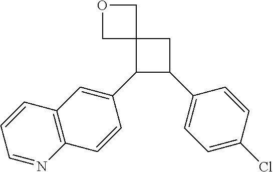 [SMILES: Clc1ccc(C2CC3(COC3)C2c2ccc3ncccc3c2)cc1]

6-(4-Chlorophenyl)-5-(naphthalen-2-yl)-2-oxaspiro[3.3]heptane (89). The title compound was synthesized according to the General Procedure C using 3-(naphthalen-2-ylmethylene)oxetane (39.2 mg, 0.2 mmol) and 1-chloro-4-vinylbenzene (83.2 mg, 0.6 mmol). The crude mixture was purified by flash column chromatography using hexanes to hexanes/ethyl acetate (20:1 v/v) as the eluent to give 58.8 mg (0.176 mmol, 88% yield, 1.4:1 d.r.) of the title compound as a colorless oil.

Major: ¹H NMR (CDCl₃, 400 MHz): δ 7.95-7.81 (m, 3H), 7.71 (s, 1H), 7.56-7.46 (m, 3H), 7.31-7.24 (m, 2H), 7.20-7.15 (m, 2H), 4.87 (d, J=6.8 Hz, 1H), 4.77 (d, J=6.8 Hz, 1H), 4.62 (d, J=6.2 Hz, 1H), 4.55 (d, J=6.2 Hz, 1H), 3.81-3.61 (m, 2H), 2.85-2.76 (m, 1H), 2.42-2.31 (m, 1H).

¹³C NMR (CDCl₃, 100 MHz): δ 142.0, 136.7, 133.5, 132.6, 132.1, 128.6, 128.5, 127.74, 127.70, 126.3, 125.9, 125.8, 125.7, 81.3, 79.1, 53.9, 45.0, 38.0, 37.2.

Minor: ¹H NMR (CDCl₃, 400 MHz): δ 7.74-7.65 (m, 2H), 7.56 (d, J=8.4 Hz, 1H), 7.46-7.35 (m, 3H), 7.03-6.97 (m, 2H), 6.89-6.78 (m, 3H), 5.03 (d, J=6.0 Hz, 1H), 4.97 (d, J=6.0 Hz, 1H), 4.55 (d, J=6.8 Hz, 1H), 4.44 (d, J=6.8 Hz, 1H), 4.17 (dd, J=9.2, 3.2 Hz, 1H), 3.99 (q, J=9.4 Hz, 1H), 3.04-2.94 (m, 1H), 2.93-2.81 (m, 1H).

¹³C NMR (CDCl₃, 100 MHz): δ 138.8, 135.4, 133.2, 132.0, 131.4, 128.5, 127.9, 127.8, 127.7, 127.6, 127.5, 126.6, 126.0, 125.5, 85.2, 79.3, 55.2, 43.5, 38.4, 36.9.

HRMS (EI) calculated for [C₂₂H₁₉ClO]⁺ requires m/z 334.1119, found m/z 334.1112.

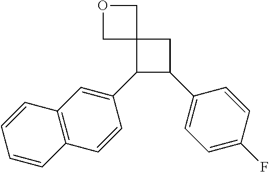 [SMILES: Fc1ccc(C2CC3(COC3)C2c2ccc3ccccc3c2)cc1]

6-(4-Fluorophenyl)-5-(naphthalen-2-yl)-2-oxaspiro[3.3]heptane (90). The title compound was synthesized according to the General Procedure C using 3-(naphthalen-2-ylmethylene)oxetane (39.2 mg, 0.2 mmol) and 1-fluoro-4-vinylbenzene (73.3 mg, 0.6 mmol). The crude mixture was purified by flash column chromatography using hexanes to hexanes/ethyl acetate (8:1 v/v) as the eluent to give 63.1 mg (0.198 mmol, 99% yield, 1.4:1 d.r.) of the title compound as a colorless oil.

¹H NMR (CDCl₃, 400 MHz): δ 7.90-7.65 (m, 3H), 7.54 (d, J=8.4 Hz, 0.57H), 7.49-7.43 (m, 1.14H), 7.43-7.35 (m, 1.43H), 7.20-7.13 (m, 0.86H), 6.99-6.68 (m, 4H), 5.03 (d, J=6.0 Hz, 0.57H), 4.96 (d, J=6.0 Hz, 0.57H), 4.83 (d, J=6.8 Hz, 0.43H), 4.72 (d, J=6.8 Hz, 0.43H), 4.61-4.54 (m, 1H), 4.51 (d, J=6.2 Hz, 0.43H), 4.46 (d, J=6.9 Hz, 0.57H), 4.14 (dd, J=9.4, 3.0 Hz, 0.57H), 3.98 (q, J=9.5 Hz, 0.57H), 3.75-3.61 (m, 0.86H), 3.03-2.93 (m, 0.57H), 2.91-2.81 (m, 0.57H), 2.80-2.71 (m, 0.43H), 2.43-2.17 (m, 0.43H).

¹⁹F NMR (CDCl₃, 376 MHz): δ −116.6, −117.3.

¹³C NMR (CDCl₃, 100 MHz): δ 161.4 (d, J=244.4 Hz), 160.9 (d, J=243.9 Hz), 139.2 (d, J=3.3 Hz), 136.7, 135.9 (d, J=3.2 Hz), 135.5, 133.5, 133.2, 132.5, 131.9, 128.6, 128.5, 127.8 (d, J=7.9 Hz), 127.7, 127.5 (d, J=6.0 Hz), 126.7, 126.2, 125.90, 125.87, 125.7, 125.5, 115.2 (d, J=21.2 Hz), 114.6 (d, J=21.2 Hz), 85.2, 81.3, 79.3, 79.1, 55.2, 54.0, 44.9, 43.4, 38.3, 37.8, 37.3, 37.0.

HRMS (EI) calculated for [C₂₂H₁₉FO]⁺ requires m/z 318.1414, found m/z 318.1407.

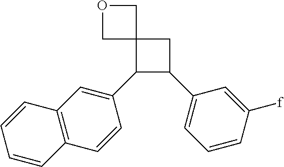

6-(3-Fluorophenyl)-5-(naphthalen-2-yl)-2-oxaspiro[3.3]heptane (91). The title compound was synthesized according to the General Procedure C using 3-(naphthalen-2-ylmethylene)oxetane (39.2 mg, 0.2 mmol) and 1-fluoro-3-vinylbenzene (73.5 mg, 0.6 mmol). The crude mixture was purified by flash column chromatography using hexanes to hexanes/ethyl acetate (8:1 v/v) as the eluent to give 59.2 mg (0.186 mmol, 93% yield, 1.4:1 d.r.) of the title compound as a colorless oil.

¹H NMR (CDCl₃, 400 MHz): δ 7.93-7.79 (m, 1.29H), 7.73-7.61 (m, 1.57H), 7.55 (d, J=8.5 Hz, 0.57H), 7.48-7.36 (m, 2.43H), 7.25-7.17 (m, 0.57H), 7.03-6.81 (m, 2.86H), 6.72-6.56 (m, 1.71H), 5.02 (d, J=6.0 Hz, 0.57H), 4.96 (d, J=6.0 Hz, 0.57H), 4.83 (d, J=6.8 Hz, 0.43H), 4.72 (d, J=6.8 Hz, 0.43H), 4.60-4.48 (m, 1.43H), 4.43 (d, J=6.9 Hz, 0.57H), 4.16 (dd, J=9.3, 3.0 Hz, 0.57H), 4.01 (q, J=9.5 Hz, 0.57H), 3.79-3.64 (m, 0.86H), 3.04-2.94 (m, 0.57H), 2.91-2.82 (m, 0.57H), 2.82-2.65 (m, 0.43H), 2.42-2.27 (m, 0.43H).

¹⁹F NMR (CDCl₃, 376 MHz): δ −113.1, −113.8.

¹³C NMR (CDCl₃, 100 MHz): δ 162.9 (d, J=246.0 Hz), 162.6 (d, J=245.3 Hz), 146.2 (d, J=7.0 Hz), 143.0 (d, J=7.1 Hz), 136.6, 135.3, 133.5, 133.2, 132.6, 132.0, 129.9 (d, J=8.4 Hz), 129.2 (d, J=8.3 Hz), 128.6, 127.71, 127.68, 127.6, 127.5, 126.5, 126.3, 125.90, 125.88, 125.8, 125.7, 125.5, 122.9 (d, J=2.8 Hz), 122.0 (d, J=2.7 Hz), 114.1 (d, J=21.3 Hz), 113.3 (d, J=21.0 Hz), 113.2 (d, J=21.0 Hz), 112.5 (d, J=21.1 Hz), 85.1, 81.3, 79.3, 79.0, 55.2, 53.8, 44.9, 43.5, 38.7 (d, J=1.8 Hz), 38.2 (d, J=1.8 Hz), 37.1, 36.7.

HRMS (EI) calculated for [C₂₂H₁₉FO]⁺ requires m/z 318.1414, found m/z 318.1415.

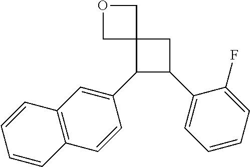 [SMILES: Fc1ccccc1C1CC2(COC2)C1c1ccc2ccccc2c1]

6-(2-Fluorophenyl)-5-(naphthalen-2-yl)-2-oxaspiro[3.3]heptane (92). The title compound was synthesized according to the General Procedure C using 3-(naphthalen-2-ylmethylene)oxetane (39.2 mg, 0.2 mmol) and 1-fluoro-2-vinylbenzene (73.1 mg, 0.6 mmol). The crude mixture was purified by flash column chromatography using hexanes to hexanes/ethyl acetate (8:1 v/v) as the eluent to give 59.7 mg (0.188 mmol, 94% yield, 1.5:1 d.r.) of the title compound as a colorless oil.

¹H NMR (CDCl₃, 400 MHz): δ 7.90-7.78 (m, 1.20H), 7.71-7.63 (m, 1.60H), 7.56-7.43 (m, 1.80H), 7.41-7.31 (m, 1.80H), 7.27-7.21 (m, 0.60H), 7.20-7.11 (m, 0.40H), 7.07-6.84 (m, 3H), 6.71-6.64 (m, 0.60H), 5.06 (d, J=6.0 Hz, 0.60H), 4.98 (d, J=6.0 Hz, 0.60H), 4.87 (d, J=6.8 Hz, 0.40H), 4.75 (d, J=6.8 Hz, 0.40H), 4.62-4.56 (m, 1H), 4.51 (d, J=6.3 Hz, 0.40H), 4.47 (d, J=6.9 Hz, 0.60H), 4.25-4.19 (m, 0.60H), 4.19-4.08 (m, 0.60H), 3.96-3.80 (m, 0.80H), 3.16-3.04 (m, 0.60H), 2.86-2.76 (m, 1H), 2.46-2.24 (m, 0.40H).

¹⁹F NMR (CDCl₃, 376 MHz): δ −115.3, −116.9.

¹³C NMR (CDCl₃, 100 MHz): δ 160.9 (d, J=245.3 Hz), 160.6 (d, J=245.1 Hz), 136.6, 135.6, 133.5, 133.2, 132.5, 131.9, 129.9 (d, J=15.5 Hz), 128.4, 128.0, 127.90, 127.89 (d, J=7.2 Hz), 127.88, 127.7, 127.64, 127.63 (d, J=8.2 Hz), 127.61, 127.4 (d, J=7.3 Hz), 127.3, 127.2, 127.1, 126.4, 126.2, 125.8 (d, J=4.7 Hz), 125.7, 125.6 (d, J=4.0 Hz), 125.3, 124.1 (d, J=3.5 Hz), 123.4 (d, J=3.4 Hz), 115.3 (d, J=22.0 Hz), 114.6 (d, J=21.5 Hz), 85.3, 81.4, 79.5, 79.1, 55.3 (d, J=1.3 Hz), 52.5, 45.3, 43.7, 37.2 (d, J=2.0 Hz), 35.4, 34.4 (d, J=1.6 Hz), 32.9 (d, J=1.6 Hz).

HRMS (EI) calculated for [C₂₂H₁₉FO]⁺ requires m/z 318.1414, found m/z 318.1406.

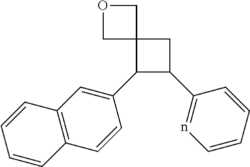

Trans-2-(5-(naphthalen-2-yl)-2-oxaspiro[3.3]heptan-6-yl)pyridine (93). The title compound was synthesized according to the General Procedure C using 3-(naphthalen-2-ylmethylene)oxetane (39.2 mg, 0.2 mmol) and 2-vinylpyridine (63 ptL, 0.6 mmol). The diastereomeric ratio of the trans- and cis-products in the crude reaction mixture was determined to be 1.1:1 based on ¹H NMR analysis. The mixture was then purified by flash column chromatography using hexanes/ethyl acetate (5:1→2:1 v/v) as the eluent to give 54.2 mg (0.180 mmol, 90% yield, >10:1 d.r.) of the title compound as a colorless oil.

¹H NMR (CDCl₃, 400 MHz): δ 8.59 (d, J=4.4 Hz, 1H), 7.90-7.78 (m, 3H), 7.71 (s, 1H), 7.57 (td, J=7.6, 1.8 Hz, 1H), 7.51-7.42 (m, 3H), 7.19 (d, J=7.6 Hz, 1H), 7.15-7.09 (m, 1H), 4.90 (d, J=6.8 Hz, 1H), 4.79 (d, J=6.8 Hz, 1H), 4.60 (d, J=6.2 Hz, 1H), 4.54 (d, J=6.2 Hz, 1H), 4.02 (d, J=9.8 Hz, 1H), 3.88-3.74 (m, 1H), 2.79-2.59 (m, 2H).

¹³C NMR (CDCl₃, 100 MHz): δ 161.7, 149.6, 137.0, 136.4, 133.5, 132.5, 128.4, 127.69, 127.66, 126.2, 125.812, 125.807, 125.6, 121.9, 121.7, 81.7, 79.2, 52.6, 44.9, 40.9, 36.2.

HRMS (EI) calculated for [C₂₁H₁₉NO]⁺ requires m/z 301.1461, found m/z 301.1467.

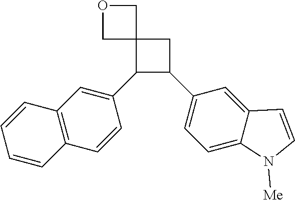 [SMILES: Cn1ccc2cc(C3CC4(COC4)C3c3ccc4ccccc4c3)ccc21]

1-Methyl-5-(5-(naphthalen-2-yl)-2-oxaspiro[3.3]heptan-6-yl)-1H-indole (94). The title compound was synthesized according to the General Procedure C using 3-(naphthalen-2-ylmethylene)oxetane (39.2 mg, 0.2 mmol) and 1-methyl-5-vinyl-1H-indole (94.3 mg, 0.6 mmol). The crude mixture was purified by flash column chromatography using hexanes/ethyl acetate (10:1→5:1 v/v) as the eluent to give 65.6 mg (0.186 mmol, 93% yield, 1.2:1 d.r.) of the title compound as a colorless oil.

¹H NMR (CDCl₃, 400 MHz): δ 7.89-7.77 (m, 1.45H), 7.74 (s, 0.45H), 7.68-7.58 (m, 1.10H), 7.54-7.40 (m, 3H), 7.38-7.29 (m, 1.10H), 7.24-7.16 (m, 0.90H), 7.10 (d, J=8.4 Hz, 0.45H), 7.01-6.83 (m, 2H), 6.68 (d, J=8.4 Hz, 0.55H), 6.38 (d, J=3.2 Hz, 0.45H), 6.29 (d, J=3.2 Hz, 0.55H), 5.04 (d, J=6.0 Hz, 0.55H), 4.98 (d, J=6.0 Hz, 0.55H), 4.86 (d, J=6.8 Hz, 0.45H), 4.75 (d, J=6.8 Hz, 0.45H), 4.63-4.51 (m, 1.45H), 4.44 (d, J=6.8 Hz, 0.55H), 4.21-4.09 (m, 1.10H), 3.85-3.75 (m, 0.90H), 3.70 (s, 1.35H), 3.56 (s, 1.65H), 3.13-3.03 (m, 0.55H), 2.95-2.85 (m, 0.55H), 2.83-2.74 (m, 0.45H), 2.51-2.30 (m, 0.45H).

¹³C NMR (CDCl₃, 100 MHz): δ 137.5, 136.3, 135.5, 135.1, 134.5, 133.7, 133.2, 132.4, 131.8, 131.1, 129.2, 128.54, 128.48, 128.3, 128.1, 127.7, 127.62, 127.57, 127.4, 127.0, 126.02, 125.97, 125.9, 125.6, 125.5, 125.1, 121.4, 120.4, 118.7, 118.0, 109.1, 108.6, 100.6, 100.4, 85.4, 81.6, 79.5, 79.4, 55.3, 54.2, 44.8, 43.7, 39.1, 38.7, 38.0, 37.2, 32.8, 32.6.

HRMS (EI) calculated for [C₂₅H₂₃NO]⁺ requires m/z 353.1774, found m/z 353.1773.

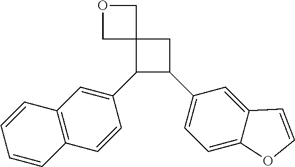 [SMILES: c1ccc2cc(C3C(c4ccc5occc5c4)CC34COC4)ccc2c1]

5-(5-(Naphthalen-2-yl)-2-oxaspiro[3.3]heptan-6-yl)benzofuran (95). The title compound was synthesized according to the General Procedure C using 3-(naphthalen-2-ylmethylene)oxetane (39.2 mg, 0.2 mmol) and 5-vinylbenzofuran (86.5 mg, 0.6 mmol). The crude mixture was purified by flash column chromatography using hexanes/ethyl acetate (10:1→5:1 v/v) as the eluent to give 62.6 mg (0.184 mmol, 92% yield, 1.0:1 d.r.) of the title compound as a colorless oil.

¹H NMR (CDCl₃, 400 MHz): δ 7.94-7.75 (m, 1.50H), 7.73 (s, 0.50H), 7.69-7.62 (m, 1H), 7.56 (d, J=2.3 Hz, 0.50H), 7.51-7.32 (m, 5H), 7.20-7.10 (m, 1.50H), 6.89 (dd, J=8.4, 1.8 Hz, 0.50H), 6.78 (d, J=8.4 Hz, 0.50H), 6.66 (d, J=2.4 Hz, 0.50H), 6.55 (d, J=2.4 Hz, 0.50H), 5.05 (d, J=6.0 Hz, 0.50H), 4.98 (d, J=6.0 Hz, 0.50H), 4.85 (d, J=6.8 Hz, 0.50H), 4.74 (d, J=6.8 Hz, 0.50H), 4.60 (d, J=6.2 Hz, 0.50H), 4.58-4.51 (m, 1H), 4.44 (d, J=6.8 Hz, 0.50H), 4.22-4.07 (m, 1H), 3.90-3.70 (m, 1H), 3.11-3.02 (m, 0.50H), 2.95-2.87 (m, 0.50H), 2.84-2.77 (m, 0.50H), 2.43-2.32 (m, 0.50H).

¹³C NMR (CDCl₃, 100 MHz): δ 153.7, 153.3, 145.3, 144.8, 138.2, 137.1, 135.8, 134.8, 133.6, 133.2, 132.5, 131.9, 128.5, 127.72, 127.71, 127.66, 127.53, 127.48, 127.4, 127.0, 126.8, 126.2, 125.93, 125.86, 125.8, 125.7, 125.3, 123.8, 122.8, 119.2, 118.5, 111.2, 110.6, 106.40, 106.36, 85.3, 81.4, 79.4, 79.2, 55.3, 54.2, 44.9, 43.5, 38.9, 38.4, 37.7, 37.2.

HRMS (EI) calculated for [C₂₄H₂₀O₂]⁺ requires m/z 340.1458, found m/z 340.1452.

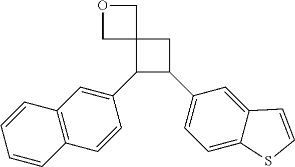 [SMILES: c1ccc2cc(C3C(c4ccc5sccc5c4)CC34COC4)ccc2c1]

6-(Benzo[b]thiophen-5-yl)-5-(naphthalen-2-yl)-2-oxaspiro[3.3]heptane (96). The title compound was synthesized according to the General Procedure C using 3-(naphthalen-2-ylmethylene)oxetane (39.2 mg, 0.2 mmol) and 5-vinylbenzo[b]thiophene (96.0 mg, 0.6 mmol). The crude mixture was purified by flash column chromatography using hexanes/ethyl acetate (10:1→5:1 v/v) as the eluent to give 62.8 mg (0.176 mmol, 88% yield, 1.0:1 d.r.) of the title compound as a colorless oil.

¹H NMR (CDCl₃, 400 MHz): δ 7.91-7.72 (m, 2.50H), 7.68-7.57 (m, 1.50H), 7.53-7.31 (m, 5H), 7.28 (d, J=5.4 Hz, 0.50H), 7.23-7.18 (m, 1H), 7.13 (d, J=5.4 Hz, 0.50H), 6.90 (dd, J=8.4, 1.8 Hz, 0.50H), 6.81 (d, J=8.4 Hz, 0.50H), 5.05 (d, J=6.0 Hz, 0.50H), 4.98 (d, J=6.0 Hz, 0.50H), 4.86 (d, J=6.8 Hz, 0.50H), 4.74 (d, J=6.8 Hz, 0.50H), 4.60 (d, J=6.4 Hz, 0.50H), 4.57-4.52 (m, 1H), 4.43 (d, J=6.8 Hz, 0.50H), 4.23-4.09 (m, 1H), 3.89-3.73 (m, 1H), 3.17-3.04 (m, 0.50H), 2.98-2.88 (m, 0.50H), 2.84-2.73 (m, 0.50H), 2.45-2.30 (m, 0.50H).

¹³C NMR (CDCl₃, 100 MHz): δ 139.8, 139.4, 137.8, 137.1, 137.0, 136.6, 135.7, 133.5, 133.2, 132.5, 131.9, 128.5, 127.8, 127.71, 127.66, 127.6, 127.5, 127.4, 126.8, 126.7, 126.20, 126.18, 125.9, 125.82, 125.78, 125.7, 125.3, 124.1, 123.62, 123.60, 123.2, 122.4, 121.74, 121.65, 120.9, 85.3, 81.4, 79.4, 79.2, 55.2, 54.0, 44.9, 43.6, 38.9, 38.4, 37.5, 37.1.

HRMS (EI) calculated for [C₂₄H₂₀O S]⁺ requires m/z 356.1229, found m/z 356.1227.

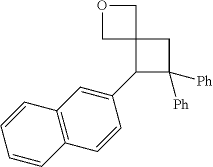 [SMILES: c1ccc(C2(c3ccccc3)CC3(COC3)C2c2ccc3ccccc3c2)cc1]

5-(Naphthalen-2-yl)-6,6-diphenyl-2-oxaspiro[3.3]heptane (97). The title compound was synthesized according to the General Procedure C using 3-(naphthalen-2-ylmethylene)oxetane (39.2 mg, 0.2 mmol) and ethene-1,1-diyldibenzene (108 mg, 0.6 mmol). The crude mixture was purified by flash column chromatography using hexanes to hexanes/ethyl acetate (20:1 v/v) as the eluent to give 62.5 mg (0.166 mmol, 83% yield) of the title compound as a colorless oil.

¹H NMR (CDCl₃, 400 MHz): δ 7.77-7.64 (m, 2H), 7.57 (d, J=8.4 Hz, 1H), 7.46-7.37 (m, 5H), 7.33-7.27 (m, 2H), 7.20-7.11 (m, 1H), 7.07-6.98 (m, 3H), 6.97-6.91 (m, 2H), 6.81 (dd, J=8.4, 2.0 Hz, 1H), 4.72-4.65 (m, 2H), 4.62 (d, J=6.4 Hz, 1H), 4.55 (d, J=6.8 Hz, 2H), 3.64 (d, J=12.6 Hz, 1H), 3.16 (dd, J=12.6, 2.1 Hz, 1H).

¹³C NMR (CDCl₃, 100 MHz): δ 150.3, 144.1, 135.6, 133.2, 132.2, 129.0, 128.4, 127.9, 127.74, 127.72, 127.44, 127.41, 127.37, 126.2, 125.89, 125.87, 125.7, 125.6, 83.9, 79.2, 59.9, 52.5, 43.8, 43.7.

HRMS (EI) calculated for [C₂₈H₂₄O]⁺ requires m/z 376.1822, found m/z 376.1812.

Stability Studies of Cu-2 and UiO-69-phen(binap)Cu

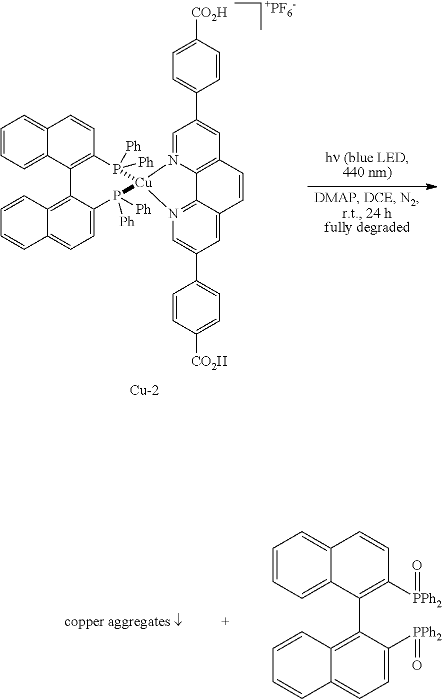

To a 25-mL flame-dried Schlenk flask cooled under N₂ were added Cu-2 (2 μmol, 2.8 mg), 4-dimethylaminopyridine (0.01 mmol, 5.0 equiv.), and 1,2-dichloroethane (1.0 mL). After three freeze-pump-thaw cycles, the reaction was irradiated by three 40-watt Kessil PR160L-440 blue-LED lamps at room temperature (with three fans) for 24 hours. 1,2-Dichloroethane was then removed in vacuo and 0.6 mL of CDCl₃ was added. The resulted mixture was analyzed by ¹H NMR and ³¹P NMR. The NMR spectra showed that Cu-2 was fully degraded; only the signal of phosphine oxide was detected by ³¹P NMR.

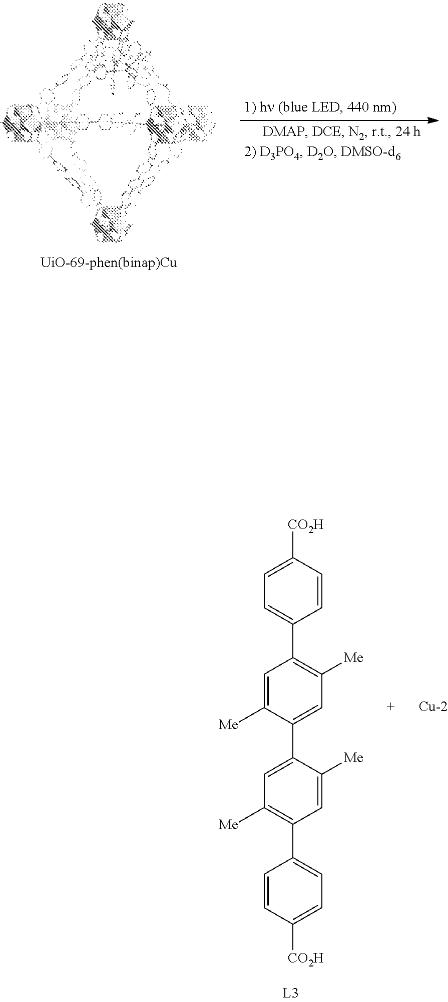

To a 25-mL flame-dried Schlenk flask cooled under N₂ were added UiO-69-phen(binap)Cu (2 μmol based on Cu, 11.9 mg), 4-dimethylaminopyridine (0.01 mmol, 5.0 equiv.), and 1,2-dichloroethane (1.0 mL). After three freeze-pump-thaw cycles, the reaction was irradiated by three 40-watt Kessil PR160L-440 blue-LED lamps at room temperature (with three fans) for 24 hours. After the irradiation, the solid was collected by filtration and washed with 1,2-dichloroethane. 2 mg of the dried solid was digested in a solution of D₃PO₄/D₂O/DMSO-d₆ (1:1:5 v/v/v) and subjected to ¹H NMR analysis. According to the ¹H NMR spectrum, Cu-2 and L3 was found to be in a ratio of 1:5.

### Recycling Experiments:

To a 25-mL flame-dried Schlenk tube cooled under N₂ atmosphere were added UiO-69-phen(binap)Cu (5.9 mg, 1 μmol based on Cu, 0.5 mol %), 4-dimethylaminopyridine (29 mg, 0.24 mmol, 1.2 equiv.), 1,2-dichloroethane (1.0 mL), 1-vinylnaphthalene (30.8 mg, 0.2 mmol), and acrylonitrile (66 ptL, 1.0 mmol) sequentially. After three freeze-pump-thaw cycles, the reaction mixture was irradiated by three 40-watt Kessil PR160L-440 blue-LED lamps at room temperature (with three fans) for 48 hours. After irradiation, the UiO-69-phen(binap)Cu was recovered by filtration, washed with anhydrous 1,2-dichloroethane (3×10 mL), and then used for subsequent cycles of the photocatalytic reaction. The filtrates were concentrated under vacuum and the yield was determined by ¹H NMR analysis. The aforementioned procedure was repeated six times.

### Mechanistic Studies:

### Triplet Excited-State Quenching Experiments

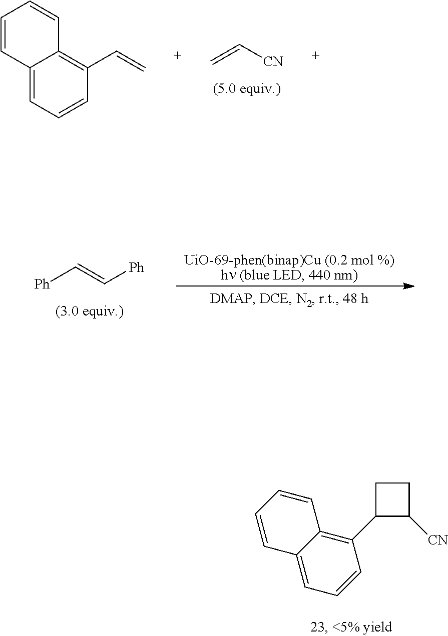

The reaction was conducted according to the general procedure A using trans-stilbene as a triplet-energy-transfer inhibitor. After the irradiation, the yield of 23 was determined by crude ¹H NMR using phenyltrimethylsilane as an internal standard. The desired intermolecular crossed cycloaddition was fully suppressed.

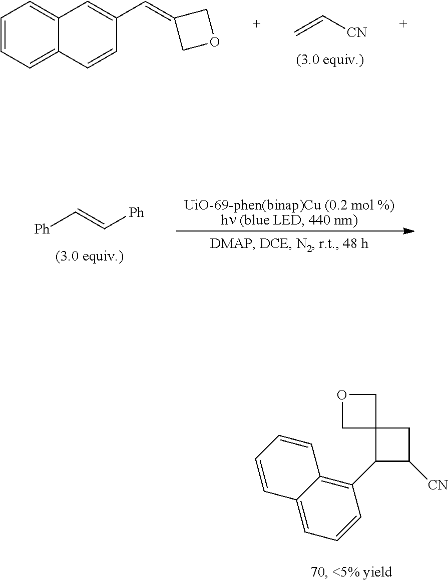

The reaction was conducted according to the general procedure C using trans-stilbene as a triplet-energy-transfer inhibitor. After the irradiation, the yield of 70 was determined by crude ¹H NMR using phenyltrimethylsilane as an internal standard. The desired intermolecular crossed cycloaddition was fully suppressed.

Kinetic Studies on the Intermolecular Crossed [2+2] Cycloaddition: For the reaction with styrene (2.0 M): to a 25-mL flame-dried Schlenk tube cooled under N₂ atmosphere were added 4-phenylmorpholine (39.2 mg, 0.24 mmol), acrylonitrile (132 μL, 2.0 mmol), and styrene (0.40 mmol) sequentially. The mixture was diluted to a total volume of 0.2 mL with 1,2-dichloroethane to obtain a homogeneous solution with a styrene concentration of 2.0 M. UiO-69-phen(binap)Cu (35.6 mg, 6 μmol based on Cu, 1.5 mol %) was added afterwards. Following three freeze-pump-thaw cycles, the reaction mixture was irradiated by three 40-watt Kessil PR160L-440 blue-LED lamps at room temperature (with three fans). 30 μL of the reaction mixture was taken out after 10, 24, 29, 34, and 48 hours, respectively. Upon solvent removal under vacuum, 0.8 mL of phenyltrimethylsilane solution (0.05 M in CDCl₃) was added to the residue, and the sample was analyzed by ¹H NMR using phenyltrimethylsilane as an internal standard.

For the reaction with styrene (1.5 M): the procedure was the same as previously described, except 0.30 mmol of styrene was added to the reaction.

For the reaction with styrene (1.0 M): the procedure was the same as previously described, except 0.20 mmol of styrene was added to the reaction.

For the reaction with styrene (0.5 M): the procedure was the same as previously described, except 0.10 mmol of styrene was added to the reaction.

For the reaction with styrene (0.2 M): the procedure was the same as previously described, except 0.04 mmol of styrene was added to the reaction.

The molar concentrations of product 16 were calculated based on the ¹H NMR integration in reference to the internal standard. At each time point, the data represented the average of two identical experiments. The molar concentrations of product 16 were plotted against the reaction time to obtain a typical reaction kinetic profile. The reaction rates were calculated by measuring the accumulation of product 16 over a 10-to-24-hour period.

### REFERENCES (CITED IN ADDITIONAL EXAMPLE 1 DETAILS ABOVE)

1. Zhang, X. et al. Catalytic chemoselective functionalization of methane in a metal-organic framework. Nat. Catal. 1, 356-362 (2018).
2. Murray, P. R. D. et al. Intermolecular crossed [2+2]cycloaddition promoted by visible-light triplet photosensitization: expedient access to polysubstituted 2-oxaspiro[3.3]heptanes. J. Am. Chem. Soc. 143, 4055-4063 (2021).
3. Wu, X.-H. et al. NH₄Cl-induced low-temperature formation of nitrogen-rich g-C₃N₄ nanosheets with improved photocatalytic hydrogen evolution. Carbon 153, 757-766 (2019).
4. Ho, W. H. et al. Proton-conductive cerium-based metal-organic frameworks. ACS Appl. Mater Interfaces 13, 55358-55366 (2021).
5. Chen, H., Wang, L.-F, Wang, J. & Yang, R. T. Investigation on hydrogenation of metal-organic frameworks HKUST-1, MIL-53, and ZIF-8 by hydrogen spillover. J. Phys. Chem. C 117, 7565-7576 (2013).

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize, or be able to ascertain using no more than routine experimentation, many equivalents to the specific embodiments of the invention described herein. Such equivalents are intended to be encompassed by the following claims.

We claim:

1. A heterogenous catalyst comprising:
a metal-organic framework formed of a plurality of inorganic nodes and a plurality of organic linkers wherein at least one organic linker of the plurality comprises a phenanthroline moiety;
wherein at least one copper (I) ion is ligated to the phenanthroline moiety and ligated to a phosphine ligand forming a catalyst complex within the metal-organic framework.

2. The heterogenous catalyst of claim 1, wherein the phosphine ligand has a chemical structure according to: 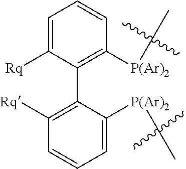
wherein Rq and Rq′ are each independently selected from hydrogen, alkyl, alkoxy, or aryl groups; and
wherein each Ar is independently selected from a phenyl or aromatic group; optionally wherein the aromatic group is 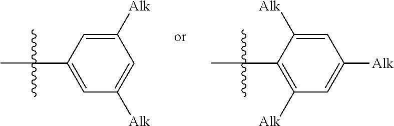
wherein each Alk is independently an alkyl group.

3. The heterogeneous catalyst of claim 1, wherein the phosphine ligand is 2,2′-bis(diphenylphosphino)-1,1′-binapthyl (binap).

4. The heterogenous catalyst of claim 1, wherein the plurality of inorganic nodes comprise [Zr₆] inorganic nodes.

5. The heterogenous catalyst of claim 1, wherein the plurality of organic linkers comprises quaterphenyl dicarboxylate organic linkers; optionally wherein the quaterphenyl dicarboxylate organic linkers comprise one or more tetramethyl substituted quaterphenyl dicarboxylate organic linkers and optionally the tetramethyl substituted quaterphenyl is 2′,2″,5′,5″-tetramethyl-[1,1′:4′,1″:4″,1″′-quaterphenyl]-4,4″′-dicarboxylic acid.

6. The heterogenous catalyst of claim 1, wherein the at least one organic linker of the plurality which comprises the phenanthroline moiety is derived from 4,4′-(1,10-phenanthroline-3,8-diyl)dibenzoic acid.

7. The heterogenous catalyst of claim 1, wherein the metal-organic framework is a Universitetet Oslo-69 metal-organic framework.

8. The heterogenous catalyst of claim 1, wherein the catalyst complex comprises a chemical structure as follows:

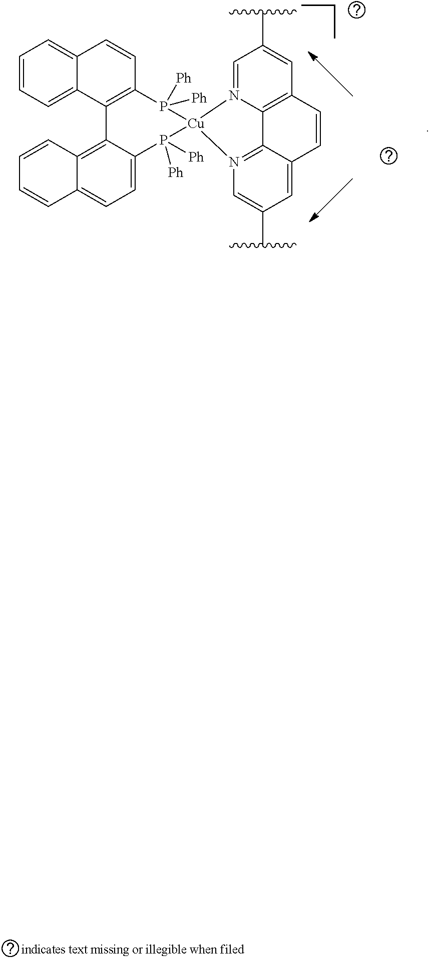

9. A method of synthesizing the heterogenous catalyst of claim 1, the method comprising the steps of:
(i) reacting a plurality of organic linkers, wherein at least one organic linker of the plurality comprises a phenanthroline moiety, with an inorganic salt to form a metal-organic framework comprising a plurality of inorganic nodes;
(ii) metalating the metal-organic framework by mixing a metal complex comprising at least one copper (I) ion ligated to a phosphine ligand with the metal-organic framework;
wherein the metalating step comprises the least one copper (I) ion becoming ligated to the phenanthroline moiety to form a catalyst complex within the metal-organic framework.

10. A method of synthesizing the heterogenous catalyst of claim 1, the method comprising the steps of:
(i′) metalating a metal-organic framework by mixing a metal complex comprising at least one copper (I) ion ligated to a phosphine ligand with the metal-organic framework;
wherein the metal-organic framework is formed of a plurality of inorganic nodes and a plurality of organic linkers wherein at least one organic linker of the plurality comprises a phenanthroline moiety;
wherein the metalating step comprises the least one copper (I) ion becoming ligated to the phenanthroline moiety to form a catalyst complex within
the metal-organic framework.

11. The method of claim 9, wherein the phosphine ligand has a chemical structure according to: 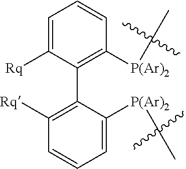
wherein Rq and Rq′ are each independently selected from hydrogen, alkyl, alkoxy, or aryl groups; and
wherein each Ar is independently selected from a phenyl or aromatic group; optionally wherein the aromatic group is 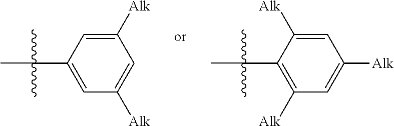
wherein each Alk is independently an alkyl group.

12. The method of claim 9, wherein the plurality of inorganic nodes comprise [Zr₆] inorganic nodes.

13. The method of claim 9, wherein the plurality of organic linkers comprises quaterphenyl dicarboxylate organic linkers; optionally wherein the quaterphenyl dicarboxylate organic linkers comprise one or more tetramethyl substituted quaterphenyl dicarboxylate organic linkers and optionally the tetramethyl substituted quaterphenyl is 2′,2″,5′,5″-tetramethyl-[1,1′:4′,1″:4″,1″′-quaterphenyl]-4,4′-dicarboxylic acid.

14. The method of claim 9, wherein the metal complex is:

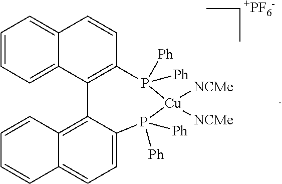

15. The method of claim 9, wherein the catalyst complex comprises a chemical structure as follows:

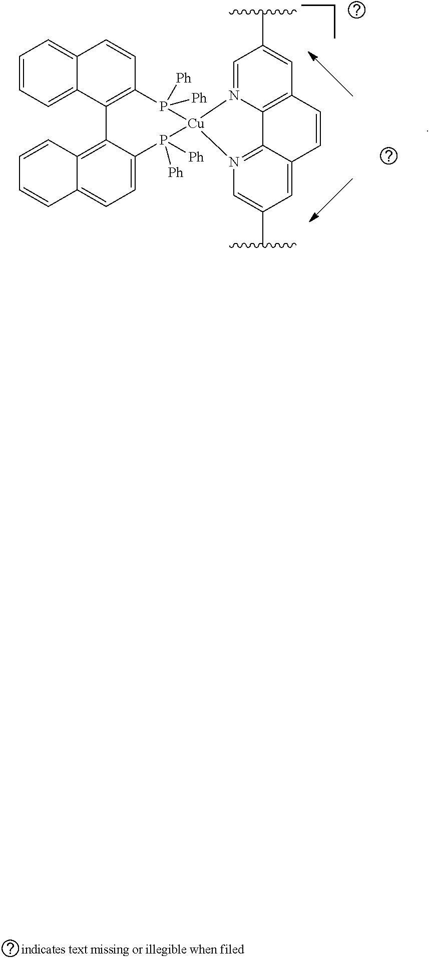

16. A method of performing a [2+2]cycloaddition comprising the steps of:
(a) forming a mixture of a first compound and a second compound in an organic solvent comprising the heterogenous catalyst of claim 1;
wherein each of the first and the second compounds comprises a carbon-carbon double bond capable of undergoing a [2+2]cycloaddition; and
(b) exposing the mixture to visible light irradiation to photoexcite the heterogenous catalyst wherein an energy transfer from the photoexcited heterogenous catalyst induces a [2+2]cycloaddition between the carbon-carbon double bonds of the first and the second compounds.

17. The method of claim 16, wherein at least one of the first and the second compounds comprises an electron deficient alkene.

18. The method of claim 16, wherein at least one of the first and/or the second compounds have a chemical structure as follows: 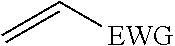
wherein EWG is an electron withdrawing group; optionally wherein the electron withdrawing group is selected from —C(O)OR; —C(O)R′; —C(O)NR₁R₂, or —CN; and
wherein R, R′, R₁, and R₂ are each independently selected from hydrogen; halogen group; a C₁-C₅ alkyl group; alkenyl group; alkynyl group; cycloalkyl group; cycloalkenyl group; cycloalkynyl group; a hydroxyl group; an aryl group; a heteroaryl group; a benzyl group; an acyl group; an ester group; a carbonyl group; a carboxylate group; an amino group; an amide group; and a nitro group.

19. The method of claim 16, wherein at least one of the first and/or the second compounds have a chemical structure according to any one of Formulae (I)-(IV): 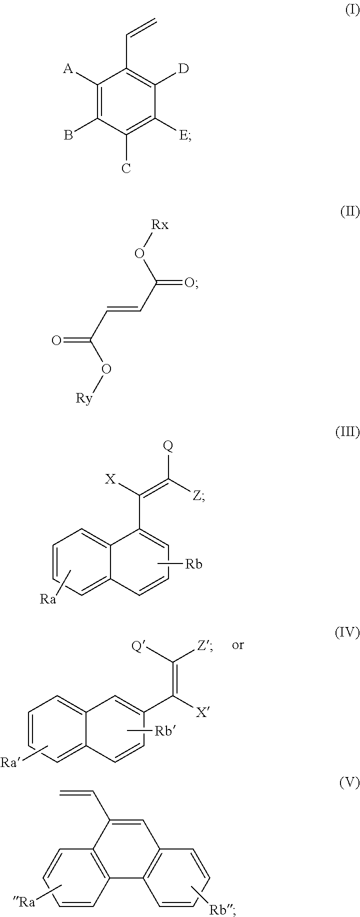
wherein A, B, C, D, and E are each independently selected from a hydrogen, halogen group; a C₂-C₅ alkyl group; alkenyl group; alkynyl group; cycloalkyl group; cycloalkenyl group; cycloalkynyl group; a hydroxyl group; an alkoxy group; an aryl group; a heteroaryl group; a benzyl group; an acyl group; an ester group; a carbonyl group; a carboxylate group; an amino group; an amide group; and a nitro group; optionally wherein A and B; B and C; C and E; and E and D may in combination form a cycloalkyl group, heterocycloalkyl group, aryl group, or heteroaryl group;
wherein Rx and Ry are each independently selected from hydrogen or an alkyl group;
wherein Ra, Ra′, Ra″, Rb, Rb′, and Rb″ are each independently selected from hydrogen, hydrogen, halogen group; a C₂-C₅ alkyl group; alkenyl group; alkynyl group; cycloalkyl group; cycloalkenyl group; cycloalkynyl group; a hydroxyl group; an alkoxy group; an aryl group; a heteroaryl group; a benzyl group; an acyl group; an ester group; a carbonyl group; a carboxylate group; an amino group; an amide group; and a nitro group;
wherein Q, Q′, X, X′, Z, and Z′ are each independently selected from hydrogen and alkyl groups; optionally wherein Q and Z and Q′ and Z′ may in combination form a cycloalkyl or heterocycloalkyl group;
wherein optionally a carbon of the aromatic rings of Formulae (I), (III), (IV), or (V) is substituted by a nitrogen.

20. The method of claim 16, wherein the visible light irradiation comprises blue light; optionally wherein the blue light comprises a wavelength of 440 nm.
